(19) United States
(12) Patent Application Publication
(10) Pub. No.: US 2025/0325658 A1
(43) Pub. Date: Oct. 23, 2025

(54) PEANUT ALLERGEN COMPOSITIONS

(71) Applicant: ALK-ABELLÓ A/S, Hørsholm (DK)

(72) Inventors: Thomas Christian Mygind, Hørsholm (DK); Kathrine Sylvestersen, Hørsholm (DK); Helene Henmar, Hørsholm (DK); Lotte Friberg, Hørsholm (DK); Peter Sejer Andersen, Hørsholm (DK); Hendrik Nolte, Hørsholm (DK); Maja-Lisa Clausen, Hørsholm (DK); Martin Rud Pedersen, Hørsholm (DK); Henrik Hugo Jacobi, Hørsholm (DK); Anne Garde Vedsø, Hørsholm (DK); Kate Hargreaves, Hørsholm (DK); Shashank Gupta, Hørsholm (DK)

(73) Assignee: ALK-ABELLÓ A/S, Hørsholm (DK)

(21) Appl. No.: 18/855,921

(22) PCT Filed: Apr. 11, 2023
(86) PCT No.: PCT/EP2023/059378

§ 371 Date: Oct. 10, 2024

(30) Foreign Application Priority Data
Apr. 11, 2022 (EP) 22167679.4

(51) Int. Cl. A61K 39/35; A61K 39/00; A61P 37/08

(52) U.S. Cl. CPC A61K 39/35; A61P 37/08; A61K 2039/545

(57) ABSTRACT

Disclosed is compositions and pharmaceutically acceptable formulations comprising the four peanut allergens Ara h 1, Ara h 2, Ara h 3, and Ara h 6, as well as a kit comprising the compositions or pharmaceutically acceptable formulations, and methods of their preparation and their use in mitigation of peanut allergy and their uses in peanut allergen-specific immunotherapy.

### FIELD OF THE INVENTION

The present invention relates to the field of immunology and in particular to active immunotherapy of allergy towards peanuts. The invention thus relates to a composition useful in active allergen-specific immunotherapy, a pharmaceutically acceptable formulation comprising the composition, a method of preparing the composition and formulation, a kit comprising the composition or formulation, and a use/method in therapy that employ the composition and pharmaceutically acceptable product.

### BACKGROUND OF THE INVENTION

Peanut allergy is an IgE-mediated immune disorder and potentially life-threatening disease with substantial impact on the quality of life of patients and their families. The clinical presentation includes a range of symptoms from oral pruritus to acute urticaria or angioedema which can progress to more serious symptoms and signs of anaphylaxis, such as anaphylactic shock, and multiple organ dysfunction syndrome. A large number of human individuals across the world are affected by peanut allergy, with the highest prevalence rates reported in the USA, Canada, UK and Australia (Pandey et al., 2019).

The raw kernels of the peanut plant Arachis hypogaea contain an array of allergens that can induce the production of specific IgE antibodies in predisposed individuals. So far, 18 proteins have been shown to bind to IgE antibodies obtained from human sera (Iqbal et al., 2016) and defined by the Allergen Nomenclature Sub-Committee of the International Union of Immunological Societies (http://www.allergen.org/). However, mainly four peanut allergens, i.e. Ara h 1, Ara h 2, Ara h 3, and Ara h 6, are considered the key IgE-binding allergens. These allergens are the most abundant peanut allergens in peanut kernels, and several studies have shown that these four allergens are clinically relevant in triggering allergic reactions (Krause et al., 2021) and have been nominated as major allergens, i.e. allergens to which more than 50% of a peanut allergic population has raised IgE-antibodies against.

The review papers of Palladino and Breitender (2018) and Becker et al. (2018) provide a thorough insight into the characteristics of the peanut allergens. The allergen Ara h 2 is considered the most important source for inducing life-threatening allergic reactions (Kukkonen et al., 2015) and Ara h 2-specific IgE and Ara h 6-specific IgE showed the greatest diagnostic accuracy for peanut allergy in comparison with other peanut allergens (Hemmings et al., 2020).

Peanut allergy has different clinical and immunologic patterns in different areas of the world. In USA, the percentage of peanut allergic patients with specific IgE antibodies against recombinantly produced Ara h 1, Ara h 2 and Ara h 3 was found to be about 80%, 90% and 60%, respectively. Conversely, significantly less peanut allergic patients were found to be sensitised towards these peanut allergens in Spain and Sweden (Vereda et al., 2011). Notably, a smaller fraction of patients is sensitised to only one of the four peanut key allergens. For example, there are reports of children with sole sensitisation to Ara h 6 without sensitisation to any one of Ara h 1, 2 and 3 (Van der Valk et al., 2016) and sole sensitisation to Ara h 3 without sensitization to Ara h 1, 2 and 6 (Restani et al., 2005).

While subcutaneous and sublingual allergen-specific immunotherapy (ASIT) of respiratory allergies have proved to be successful in developing tolerance/sustained unresponsiveness to the offending allergens after end of ASIT, these treatment options are not available for clinical use to treat food allergy. Subcutaneous allergen-specific immunotherapy (SCIT) with aqueous peanut extract has been found to be too dangerous in terms of inducing severe allergic reactions including anaphylaxis, but alternatives such as sublingual (SLIT), epicutaneous (EPIT), and oral immunotherapies (OIT) have been tested and found to provide desensitisation to a varying degree while on therapy. Sustained unresponsiveness or tolerance after treatment cessation has yet to be demonstrated, although OIT with high maintenance dose of 4000 mg peanut protein or prolonged low dose maintenance treatment with 300 mg peanut protein seemed to increase the likelihood that a subgroup of patients could remain unresponsive after discontinuation of the treatment for a minor period (Chinthrajah et al., 2019).

The mode of action of allergen-specific immunotherapy is not fully understood and different immune reactions are considered, of which B cell isotype switching from the production of IgE towards IgG (including the subclasses IgG1 and IgG4) and IgA seems important. The reduced IgE levels following ASIT would limit the IgE-mediated activation of mast cells and basophils and IgE-facilitated antigen presentation and Th2 cell responses. The increase in peanut-allergen specific IgG4- and probably also IgA antibodies would compete with IgE for allergen binding and may inhibit the formation of allergen-IgE complexes, which otherwise would bind to surface low-affinity receptor for IgE (FcεRII) on B cells resulting in IgE-facilitated T_{H}2 cell development (Durham and Shamji, 2022). Peanut OIT have been observed to result in modified levels of IgE and IgG4 antibodies (Vickery et al. 2013).

Recently, the FDA has approved a standardised peanut powder for oral administration for conducting oral immunotherapy (OIT) to mitigate peanut allergy, including anaphylaxis, that may occur with accidental exposure to peanut (Palforzia®). The peanut powder is manufactured from defatted lightly roasted peanut flour and each dose meets specifications for quantities of Ara h 1, Ara h 2 and Ara 6 measured by immunoassay or in combination with high performance liquid chromatography (FDA package leaflet of Palforzia®). The powder must be stored refrigerated at 2° C. to 8° C. and mixed with food prior to ingestion. Patients diagnosed with peanut allergy ingest the peanut flour mixed with food in a gradual updosing scheme, including initial-day dose escalation from 0.5 mg to 6 mg peanut protein in a health care setting, followed by a build-up phase consisting of 11 dose levels of 2-week periods of daily administration of same dose, starting from 3 mg peanut protein to 300 mg peanut protein (100 times increase in dose over five months). Finally, treatment is continued with a maintenance phase with 300 mg peanut protein daily. According to the FDA package leaflet of Palforzia®, there is a risk of developing anaphylaxis and to get gastrointestinal adverse events during treatment. In a phase III clinical trial, it was found that tolerance to peanut increased from a threshold from <100 mg peanut protein at baseline to about 600 to 1000 mg after 50 weeks of OIT treatment. However, a high number of dropouts (11.6% active; 2.4% placebo) were reported due to adverse events (AEs). Importantly, the use of epinephrine to mitigate anaphylaxis was reported in 14.0% of the individuals in the active group compared to 6.5% in the placebo group (Vickery et al., 2018). Thus, OIT seems to carry a higher risk of developing an anaphylactic shock compared to not-treated patients.

Koppelman et al. (2018) have found that the concentration of allergens (Ara h 1, 2, 3 and 6) extractable in solubilised form from lightly roasted peanut flour varies significantly with pH. In the pH range of saliva from 6.5 to 8.5, the Ara h 3 solubility increases significantly, while the remaining allergens are much less affected. Overall, the extraction kinetics in this pH range suggested that Ara h 2 and Ara h 6 are the first allergens an individual is exposed to upon ingestion of peanut and that the extracted amount of Ara h 1 in nmol/ml was rather low compared to the other allergens when determined in the pH range of saliva (6.5 to 8.5).

Various compositions and dosing schedules have been suggested for OIT against peanut allergy: The patent applications WO2014159609 and WO2014159607 relate to peanut protein compositions comprising flour of roasted peanuts, and WO2014159609 further relates to OIT dosing schedules with initial 1-day dose escalation followed by nine bi-weekly updosing steps from a dose of 12 mg peanut protein to 300 mg peanut protein. The patent application WO2016020336_relates to peanut protein compositions suitable for oral administration wherein the peanut allergens are released in the stomach. The patent application US2020038466 relates to peanut protein compositions derived from roasted peanuts for use in oral immunotherapy against peanut allergy, wherein it is suggested to use compositions comprising Ara h 1 in an amount between 10-15% by weight of the total proteins, Ara h 2 in an amount between 2-10% by weight of the total proteins and Ara h 3 between 10-20% by weight of the total proteins.

SLIT has been investigated in smaller human trials by administration of liquid drops of a peanut allergen extract in a dosing regimen comprising a lengthy updosing phase. In a trial reported by Kim et al. (2011), children were treated daily with aliquots of a liquid peanut allergen extract that contained 5000 μg/mL of peanut proteins (estimated to contain 300 μg/mL of Ara h 2, corresponding to 6% by weight of peanut protein) dissolved in 0.2% phenol and 50% glycerinated saline (obtained from Greer Laboratories). The allergen extract was administered sublingually in increasing volumes (drops) over a period of approximately six months followed by six months maintenance phase with once daily dose of 2000 μg peanut protein (six percent is Ara h 2 corresponding to 120 μg Ara h 2). The updosing phase comprised 14 bi-weekly dose-escalation steps from 0.25 μg of peanut protein (0.0015 μg of Ara h 2) to 2000 μg peanut protein (about 120 μg Ara h 2). Thus, during the updosing phase, the dose was increased 8000 times over six months. It was found that the children randomised to active peanut SLIT could ingest a median cumulative dose of 1,710 mg of peanut protein (equivalent to 6-7 peanuts) in an oral challenge test compared to the seven children receiving placebo, who ingested a median cumulative dose of 85 mg peanut protein. Burk et al. (2015) later reported from a continuation of the trial that ten of 33 children (30%) completed a 2500 mg peanut protein cumulative oral food challenge without symptoms, while the remaining children tolerated a median of only 460 mg peanut protein (10-1710 mg). The methodology of conducting an oral food challenge is described by Sampson et al. (2012). In another SLIT trial reported by Fleischer et al. (2013), liquid allergen extract obtained from Greer Laboratories (allergenic extract from non-roasted peanut with 0.5% sodium chloride and 0.54% sodium bicarbonate at a pH of 6.8-8.4 as aqueous extracts in 50% glycerine) was administered in a 36-week updosing phase with a start dose of 0.000165 μg of peanut protein (0.00001 μg of Ara h 2) and an end dose of 1386 μg peanut protein (about 83 μg Ara h 2), which was used in the following maintenance phase. Some patients were further updosed to 3696 μg peanut protein (about 222 μg Ara h 2) after 8-28 weeks of maintenance dosing with 1386 μg peanut protein and continued maintenance phase with the 3696 μg peanut protein dose. An overall comparison on SLIT and OIT has been published by Zhang et al. (2018).

SLIT with co-administration of a TLR4 agonist (e.g. a glucopluranosyl lipid adjuvant) along with peanut allergen(s) has been suggested to modulate allergen-specific immune responses (patent application WO2016/172511), and a clinical trial is reported to assess the tolerability and safety of a peanut extract (PE) adjuvanted with Glucopyranosyl Lipid A (GLA) after repeated sublingual (SL) daily administration in peanut allergic adult and adolescent patients (ClinicalTrials.gov Identifier: NCT03463135).

SLIT relies on the delivery of soluble allergens to the sublingual mucosa in a conformational form that ensures efficient entry into the oral mucosa and uptake by antigen-presenting cells. Where SLIT is conducted by the administration of solid dosage forms (such as lyophilised dosage form), the allergens must be released from the solid dosage form and dissolved in the saliva to become soluble and bio-accessible.

Usually, allergen products for ASIT are based on allergen extracts containing a high diversity of allergens in their native conformation, including various isoforms and post-translational modifications (e.g. glycosylation). The use of natural forms of the allergens may ensure that the allergic patient is treated with allergens comprising all the potential IgE-antibody binding epitopes that a patient or a population of patients might have raised IgE against upon natural exposure to the allergen-source material. Therefore, allergen products based on recombinantly produced allergens may not cover all the native IgE-epitope binding sites and the recombinant allergen may not exist in the “natural” post-translational conformation.

Allergens are proteinic molecules known by their amino acid sequences. Allergens exist in various isoforms and may be modified by post-translational processes following expression. Notably, the peanut allergens Ara h 1 and Ara h 3 seem to be present in raw peanuts in oligomeric forms, whereas Ara h 2 and 6 seem to exist merely in monomeric form (Boldt et al., 2005). When purified from raw peanut, Ara h 1 is reported to be available as a stable 210 kDa trimeric protein composed of 63 kDa N-glycosylated subunits, which can form multimers of up to 600-700 kDa depending on extraction conditions (Blanc et al., 2011). However, the existence of Ara h 1 in oligomeric form rather than in monomeric form may depend on the methods used for purification as indicated by the work of van Boxtel et al. (2006). The allergen Ara h 3 is a complex allergen consisting of a single-chain polypeptide (monomeric form) of about 60 kDa, and less stable to enzymatic (e.g. pepsin) action than the Ara h 2 and Ara h 6 allergens. It has a molecular mass of about 60 kDa for the monomeric form based on the amino acid sequence, but Ara h 3 seems to occur in peanuts as a hexameric heteromeric complex of 360 kDa, which post-translationally is cleaved into a 43 kDa acidic and a 28 kDa basic subunit that are covalently linked by a disulfide bond. Several fragments of Ara h 3 (14, 25, 42 and 45 kDa) can be observed, even under extraction conditions that inhibit protease activity (Palladino and Breitender, 2018). If proteolytically processed, Ara h 3 would be bound by disulphide bridges and is found in trimeric and hexameric structures. Ara h 3 is also known to easily aggregate into complex polymers during roasting, and Ara h 3 from roasted peanut has in contradiction to Ara h 3 from raw peanut been shown to led to an increase in the uptake of Ara h 3 in Caco-2 cells, probably due to the higher quantity of Ara h 3 being absorbed in cells in aggregated form (Wang S et al. 2021). An allergen with molecular size of 35.9 kDa and with a pI of 5.5 is found with 91% identical amino acid sequence with Ara h 3, and is considered an isoalleren of Ara h 3, although sometimes referred to as Ara h 4.

The intrinsic allergenicity of an allergen may be altered through protein aggregation, e.g. via disulfides or other interchain covalent bonds, because the structural changes at the protein level may result in the disappearance and/or appearance of new IgE-binding epitopes. In addition, the conformational changes induced by protein aggregation may result in the formation of protein aggregates or oligomers with decreased solubility (De Angelis et al., 2018).

The allergen profiles that patients might be exposed to upon administering aqueous extracts of peanut may also depend on the peanut source in that peanut exists in many cultivar variants. Although Koppelman et al. (2016) did not find huge differences in the allergen profiles obtained from extraction of raw peanuts from the most predominant peanut cultivars: Runner, Virginia, Spanish, and Valencia, on the contrary, Pandey et al. (2019) found huge variations, such as 1000 fold difference, in the content of each of the four allergens Ara h 1, 2, 3 and 6 between 264 different peanut cultivar variants when extracted in aqueous buffer at pH 7.4. The allergen content was measured by sandwich ELISA. According to Koppelman et al. (2016), which made the quantitative analysis of the four allergens by RP-HLPC, it was estimated that the content by weight to total peanut protein of Ara h 1 ranged from 11.7 to 23.7%, with a mean of 17.1%±3.4%; the content of Ara h 2 ranged from 3.5 to 8.0% with a mean of 6.2%±1.3%, the content of Ara h 3 ranged from 57.7 to 83.5% with a mean of 70.6±8.6% and the content of Ara h 6 content ranged from 2.5 to 9.7% with a mean of 5.8±1.8%. Further variation in the allergen profiles occurs due to different genotypes of each peanut cultivar variant. The differences in the allergen profiles of raw peanuts as determined by Pandey et al. (2019) and Koppelman et al. (2016) may be explained by the analytical method used for determining the quantitative content of the allergens.

According to Maleki et al. 2010, high-molecular weight proteinic structures may be found to a varying degree in commercial liquid peanut extracts (ALK-Abelló, Hollister-Stier and Greer extracts) and in aqueous extracts of roasted and boiled peanuts. Such structures may be identified as smears at the top of SDS-PAGE or may be identified by Western blot analysis using binding to serum IgE of patients. Although the major peanut allergens (Ara h 1, Ara h 2, Ara h 3, and Ara h 6) were present in all extracts, the serum IgE of individual peanut allergic patients only recognised these allergens in some of the extracts. Therefore, even though the allergens are present in each extract, the allergens of each extract are not recognised by serum IgE of the same patient. In addition, the high-molecular weight proteinic structures were only recognised by IgE of some human sera.

According to Poms et al. 2004, the most significant factor affecting the extraction efficiency of peanut proteins from peanuts appeared to be the pH of the employed extraction buffer. The best total protein yield was obtained with buffers in the range of pH 8-11. However, proteins from roasted peanuts that were extracted by the same buffers resulted in considerably lower yields compared to raw peanuts. Therefore, it is challenging to provide compositions of peanut allergen extracts, which would deliver reproducible and clinically relevant doses of the allergens for peanut ASIT in that it among other parameters would require peanut allergen compositions containing the key allergens in the natural conformation which are recognised by peanut-allergen specific IgE of a majority of allergic patients.

In summary, there is a need for providing allergen-extract based products for use in peanut-specific ASIT, which contain easily soluble allergens present in their natural conformation to ensure high solubilisation of the allergens when administered in solid dosage forms to the sublingual mucosa, and further to ensure that the allergen in the natural conformation is recognised by peanut-allergic patients.

Advantageously, peanut allergen compositions containing all the four key peanut allergens would have the potential to treat a worldwide population of peanut allergic individuals independently of their sensitisation pattern. Further, by adjusting the molar content of each of the peanut allergens Ara h 1, 2, 3, and 6 to be within the same narrow molar range, any patient sensitised to either Ara h 1, 2, 3 or 6 would have the potential of being treated equally in the sense that the patient would be exposed to the same number of molecules of each of the four peanut allergens in question during treatment.

Therefore, it is important to provide peanut allergen-containing products for use in ASIT with the peanut allergens Ara h 1, 2, 3, and 6 in controlled amounts, hereunder to provide methods for manufacturing peanut allergen extracts containing the key allergens within the same and reproducible molar range.

Current allergen products are controlled by determining the total allergenic potency, either by use of a company-specific in-house reference allergen compositions that is quantified by skin test reactivity (in vivo standardisation) or by use of solid phase reference allergen extracts (FDA). Further, single allergens may be controlled by competitive in-vitro IgE tests, such as RAST, ImmunoCAP, or ELISA inhibition assays, but the biological potency measured by these methods may neither correlate with the protein content nor the amount by weight of the single allergens. The biological potency would have a dependency on the IgE-epitope coverage of the human sera or monoclonal antibody used for determining the biological potency and may not reflect the absolute amount of the single allergen in the allergen product. The use of absolute quantification of allergens by Mass Spectrometry (MS) for defining and characterising allergenic extracts is recommended by Spiric et al. (2017) to prove consistency among consecutive batches of the allergen extracts or allergen source materials. However, Quantitative MS assays based on quantification of unique peptides of the protein might fail in detecting loss in potency due to denatured proteins or partial digestion of proteins. The patent application WO2017115139 relates to the MS analysis of peanut allergens including various isoforms of each peanut allergen based on quantification of unique peptides of peanut allergens.

Therefore, additional methods for controlling the key allergens in an allergen-extract based product are required to secure high batch-to-batch consistency in the levels of single allergens, which further is important for obtaining low batch-to-batch variation of the allergenic potency.

The patent application WO2022147173A1 relates to peanut protein compositions for use in treating peanut allergy, said peanut compositions are formulated as nano-emulsions for administering peanut allergens in low concentrations.

The patent application EP3244212A1 relates to compositions of recombinantly produced peanut allergens for the application in a diagnostic test strip.

The patent application US 2018/044384A1 relates to compositions containing recombinant bacterial spores expressing Cholera toxin B (CTB) together with one or more peanut allergens on the surface of the bacterial spores/cells and methods for using such compositions for inducing tolerance or reducing sensitivity to a peanut allergen or peanut allergy

The patent application US 2005/063994 relates to compositions comprising microorganisms containing a recombinant version of peanut allergens.

Marsh et al. (2008) relates to methods for purifying individual peanut allergens. The methods comprise several chromatographic steps, of which anion exchange chromatography is used in combination with other chromatographic affinity steps for the purification of some of the peanut allergens.

Wunschmann S et al. (2019) relates to the quantitative determination of peanut allergens by ELISA, where the peanut allergens are extracted from roasted and defatted peanut flour.

### OBJECT OF THE INVENTION

It is an object of embodiments of the invention to provide compositions useful in allergen-specific immunotherapy against peanut allergy as well as to provide methods suitable for the preparation of such compositions and methods and kits that implement the compositions.

### SUMMARY OF THE INVENTION

It has been found by the present inventor(s) that aqueous extraction methods conventionally used for obtaining allergen compositions eligible for ASIT would not be feasible for providing allergen extracts suitable for peanut allergen immunotherapy. First, it is considered key to treat with high doses of all four key peanut allergens to induce clinically relevant tolerance to all four allergens. In other words, it is key to obtain reduced levels of peanut allergen-specific IgE-antibodies and increased levels of peanut allergen-specific IgG4 antibodies against all four key peanut allergens.

Unfortunately, the optimal extraction efficiency among the four key peanut allergens cannot be obtained under the same extraction conditions. The extraction efficiency of Ara h 3 is very sensitive to pH and salt concentrations and it appears that high extraction efficiency of Ara h 3 is in contradiction to high extraction efficiency of Ara h 1. Furthermore, it has been found that peanut allergen compositions obtained by simple aqueous extraction of peanut kernels contain high molecular mass proteins, which seem to be aggregates comprising nAra h 1 and/or nAra h 3 polypeptides. The aggregates may at first sight seem solubilised in the aqueous solution but may precipitate out upon storage or may cause gelation. The aggregates may also provide problems in terms of determining the accurate levels of the allergens in their single polypeptide form (monomeric form), eventually in their water-soluble oligomeric forms, or the allergen potency in the compositions. Immuno-chemical methods for determining allergen potency (e.g., ELISA) require antibodies specific for the allergens and may erroneously also bind to the aggregated allergens.

Surprisingly, the present inventors have provided compositions comprising each of the four allergens nAra h 1, nAra h 2, nAra h 3 and nAra h 6 with limited content of high molecular mass aggregates. Such compositions can be obtained by a simple few-step preparation process, which essentially comprises the aqueous extraction of peanut kernels to obtain dissolved peanut allergens nAra h 1, nAra h 2, nAra h 3 and nAra h 6, which can be adsorbed to anion exchange chromatography material and collected into individual fractions enriched for either of the allergens nAra h 1, nAra h 2, nAra h 3 and nAra h 6 following elution of the anion exchange material with different salt concentrations. The enriched fractions can be mixed to obtain pre-selected concentrations of two or more of the four key allergens.

Advantageously, this preparation method allows for the generation of compositions with similar high doses of all the four key allergen including Ara h 3 and Ara h 1.

The present inventors have also found that the concentration of each of the four allergens including nAra h 3 and nAra h 1 in the enriched fractions or mixed compositions thereof can be accurately controlled by reverse phase chromatography due to the absence of high mass aggregates. The concentration determination of nAra h 1 and nAra h 3 is particularly challenging due their existence in oligomeric forms, but the inventors have found that the reverse phase chromatography method was able to determine the concentration of each of the four allergens expressed as monomeric conformation, even though the enriched fractions or mixed compositions thereof contain nAra h 1 in trimeric form and nAra h 3 in monomeric, trimeric as well as hexameric form. Advantageously, this allows the determination of the molar concentration of each of the allergens in terms of the molar concentration of the single polypeptide form (monomeric) of the allergens.

Therefore, it has been possible to provide compositions comprising each of the allergens nAra h 1, nAra h 2, nAra h 3 and nAra h 6 in pre-selected concentrations, which by choice comprise balanced amounts of the four key allergens, which, within some boundaries, are meant to include compositions comprising the four key allergens in similar molar amounts.

When applying such compositions with similar molar concentration of the key peanut allergens in allergen-specific immunotherapy for mitigating peanut allergy, the compositions would have the potential to treat a larger fraction of peanut allergic individuals independent of their sensitisation pattern (whether being either mostly directed to nAra h 2 and/or one or more of the other key allergens) as any patient will be exposed to the same number of molecules, though within some boundaries, of each of the key peanut allergens nAra h 1, 2, 3, and 6. Thus, the mitigation of peanut allergy by use of such compositions might be eligible for many allergic patients independently of their individual peanut allergen sensitisation patterns. Moreover, if such compositions are used in a dose escalation treatment regimen (updosing), the increase in the dose of each of the key allergens will result in the same narrow dose range of each of the key allergens at each escalation step. This might increase the chance that the patient is treated with an effective dose of each of the key allergens, and advantageously is reached within the same period. Also, it is considered equally important that the use of the compositions described herein for mitigation of peanut allergy would result in the induction of the protective peanut allergen specific IgG4 antibodies, and preferably to obtain an increase in the peanut allergen-specific IgG4 antibodies for all four key peanut allergens. Advantageously, the increase in the peanut allergen-specific IgG4 might be within the same multi fold increase of the specific IgG4 antibodies recognising Ara h 1, 2, 3 as well as 6, respectively. The ability to increase the levels of IgG4 antibodies might be investigated in a human trial and the IgG4 antibodies might the quantified from blood samples or other biological secretes like saliva, nasal or lung lavages. Mice models can also be used to investigate the ability to induce IgG antibodies following allergen exposure in so far that mice do not produce IgG4 antibodies but rather IgG2 antibodies in response to allergen exposure.

According to a 1^{st} aspect of the present invention, a composition is provided comprising each of the peanut proteins nAra h 1, nAra h 2, nAra h 3, and nAra h 6, wherein the molar ratio of each of the pairs nAra h 1:nAra h 2, nAra h 3:nAra h 2 and nAra h 6:nAra h 2 is in the range of 0.5 to 2.0, preferably 0.5 to 1.5.

In a 2^{nd} aspect, the present invention relates to a pharmaceutically acceptable formulation, such as a pharmaceutical composition), wherein the formulation comprises a composition of the first aspect of the invention, or any embodiments thereof disclosed herein, which is dissolved or dispersed in a carrier substance selected from the group consisting of a liquid, a semi-solid, and a solid carrier substance.

In a 3^{rd} aspect, the present invention relates to the composition of the 1^{st} aspect of the invention or (any embodiments thereof disclosed herein) or the pharmaceutically acceptable formulation of the 2^{nd} aspect of the invention (or any embodiments thereof disclosed herein) for use as a medicament, and in particular for use in a method of treating a human against peanut allergy, such as by conducting peanut allergen-specific immunotherapy. Also within this aspect is a composition for use in the method of the 6^{th} aspect of the invention described infra.

In a 4^{th} aspect, the present invention relates to a method for preparing a composition comprising two or more of nAra h 1, nAra h 2, nAra h 3, and nAra h 6, the method comprising providing 1) an extract of peanut protein obtained by extracting raw peanut kernels with an aqueous solvent to obtain an aqueous extract comprising each of nAra h 1, nAra h 2, nAra h 3, and nAra h 6; and 2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 is eluted and collected into fractions individually enriched for nAra h 1, nAra h 2, nAra h 3, or nAra h 6; and 3) optionally collecting a flow-through fraction from the anion exchange chromatography; and 4) combining two or more fractions or aliquots thereof as obtained in step 2 or in combined steps 2 and 3 to obtain said peanut protein composition comprising at least two of nAra h 1, nAra h 2, nAra h 3, and nAra h 6. Preferably, fractions containing peanut protein with high molecular mass have been discarded.

In a 5^{th} aspect, the present invention relates to a kit comprising a sealed package comprising a plurality of separate compartments, each compartment comprising a unit dose form of the pharmaceutically acceptable formulation of the 2^{nd} aspect of the invention (or any embodiments thereof disclosed herein), wherein at least one unit dose form comprises an amount of total peanut allergen, which is non-identical with the amount in another unit dose form in the kit.

In a 6^{th} aspect, the present invention relates to a method of treating a human against peanut allergy, such as by conducting peanut allergen-specific immunotherapy, the method comprising administration of one daily dose of a composition of the 1^{st} aspect of the invention (or any embodiments thereof disclosed herein) or of the pharmaceutically acceptable formulation of the 2^{nd} aspect of the invention (or any embodiments thereof disclosed herein) over a prolonged period of time.

Also within the scope of the 6^{th} aspect is method of treating a human by allergen-specific immunotherapy against peanut allergy, the method comprising an updosing phase and optionally a maintenance phase, wherein the updosing phase comprises multiple consecutive series of administering of a daily dose of peanut protein composition to the oral mucosa, wherein the daily dose within each series is identical and wherein any dose in a preceding series is lower than in a subsequent series and wherein each series has a duration length ranging from 6 to 30 days; and wherein the daily dose administered in the first series contains a total amount of peanut protein in the range of 0.1 μg to 200 μg; the daily dose of the last series contains a total amount of peanut proteins in the range of 300 μg to 5000 μg; and wherein the number of series is in the range from 2 to 9, such as in the range of 3 to 7, such as particularly, 3, 4, 5, 6, 7, 8, or 9, preferably 3, 4, or 5. The peanut protein is extracted or extractable from raw peanut kernels by an aqueous solvent having p in the range of 7 to 9 and result in the extracted peanut proteins at least comprising each of nAra h 1, nAra h 2, nAra h 3, and nAra h 6.

### LEGENDS TO THE FIGURES

For all figures showing chromatograms of SEC HPLS analysis, the size-indicating calibration standards are shown in light grey colour and the sample as analysed is shown with black line.

FIGS. 1a and 1b show allergen profile of the pure standards of nAra h 1 as evaluated by SEC HPLC analysis (FIG. 1a) or by RP-HPLC (long-run method) (FIG. 1b).

FIGS. 2a and 2b show allergen profile of the pure standards of nAra h 2 as evaluated by SEC HPLC analysis (FIG. 2a) or by RP-HPLC (long-run method) (FIG. 2b).

FIGS. 3a and 3b show allergen profile of the pure standards of nAra h 3 as evaluated by SEC HPLC analysis (FIG. 3a) or by RP-HPLC (long-run method) (FIG. 3b).

FIGS. 3c and 3d show the profile of nAra h 3 hexameric form as evaluated by SEC HPLC analysis (FIG. 3c) or by RP-HPLC (long-run method) (FIG. 3d).

FIGS. 3e and 3f show the profile of nAra h 3 trimeric form as evaluated by SEC HPLC analysis (FIG. 3e) or by RP-HPLC (long-run method) (FIG. 3f).

FIGS. 3g and 3h show the profile of nAra h 3 monomeric form as evaluated by SEC HPLC analysis (FIG. 3g) or by RP-HPLC (long-run method) (FIG. 3h).

FIGS. 4a and 4b show allergen profile of the pure standards of nAra h 6 as evaluated by SEC HPLC analysis (FIG. 4a) or by RP-HPLC (long-run method) (FIG. 4b).

FIG. 5 shows a photo of the defatted peanut source material collected directly from the “screw press” and following gently crushing into “flakes” (to the left) and photo of a typical batch of the final powdered defatted peanut source material (to the right).

FIGS. 6a and 6b show the relative extraction efficiencies among the four peanut allergens Ara h 1, 2, 3 and 6 as a function of pH (pH range 5 to 9) and as a function of NaCl concentrations (0 to 1000 mM).

FIG. 7 shows the IgE inhibition curve performed on serum obtained from a US donor. Blue is roasted peanut extract as bound antigen and roasted peanut extract as free antigen (inhibitor). Red is roasted peanut extract as bound antigen and non-roasted peanut extract as free antigen. Orange is non roasted peanut extract as bound antigen and roasted peanut extract as free antigen. Black is non-roasted peanut extract as bound antigen and non-roasted peanut extract as free antigen.

FIGS. 8a-e show the RP-HPLC profiles of allergen extracts of different Runner genotypes: a) Arabu crude extract of genotype #3321 (collected at Arabi, GA); b) Alamo crude extract of genotype #3321 (collected at Alamo, GA), c) Chula crude extract of genotype #1041 (collected at Chula, GA), d) McRae lot #1 crude extract of genotype #212C (collected at McRae, GA), and e) McRae lot #2 crude extract of genotype #1041 (collected at McRae, GA). The HPLC profiles are slightly different. However, there are differences in the peak profiles in the part of the chromatograms where Ara h 3 appears. The genotype #1041 (McRae) seemed to cover the most isoforms.

FIGS. 9a and B show the RP-HPLC allergen profile (long run method) of a peanut extract made with extraction buffer A, pH 7.4 (FIG. 9a) versus buffer B, pH 8.5 (FIG. 9b) FIGS. 10a-10d show the RP-HPLC chromatograms (short run) of fractions A, B, C, D and the flow-through fraction (FT) collected from the anion exchange chromatography during fractionation of a peanut allergen extract. fraction A is enriched with Ara h 6 (FIG. 10a), fraction B is enriched with Ara h 2 (FIG. 10b), fraction C is enriched with Ara h 2 (FIG. 10c), fraction D is enriched with Ara h 3 (FIG. 10d) and fraction FT is not enriched with either of the allergens nAra h 1, 2, 3, and 6 (FIG. 10e).

FIGS. 10f to 10i show the RP-HPLC chromatograms (short run) of the pure reference standards of nAra h 1, 2, 3, and 6.

FIG. 11 shows the RP-HPLC chromatogram (short run method) of a purified allergen extract made by mixing the FT fraction and appropriate volumes of fractions A, B, C and D to produce an extract with balanced molar amounts of each of the allergens nAra h 1, 2, 3, and 6 and with content of minor allergens present in the FT fractions.

FIGS. 12a-b show the relative molar content of Arah 1, 2, 3, and 6 normalised to Ara h 2 after quantification of each of the allergens by LC-MS/MS. FIG. 12a illustrates the profile of three batches (A, B, and C) of peanut allergen compositions obtained in Example 5 and FIG. 12b illustrates the profile of four commercially available batches of a comparator allergen extract (Greer SPT product).

FIG. 13 shows RP-HPLC overlay chromatogram of purified allergen extract obtained by a process comprising anion exchange chromatography (DS) and a commerciable available peanut allergen extract (SPT of Greer).

FIG. 14 shows the SEC analysis (X300 column) of crude allergen extract obtained in Example 3.

FIG. 15 shows the SEC analysis (X300 column) of purified allergen extract obtained in Example 5.

FIG. 16 shows SEC analysis (X300 column) of a comparator allergen extract (Greer SPT product).

FIG. 17 shows SEC analysis (X300 column) of three batches (A, B and C) of purified allergen extracts obtained by the same manufacturing process according to examples 2 to 5. Dotted lines shows the standards nAra h 1 and nAra h 3.

FIG. 18 shows a flow chart of the entire process for manufacturing peanut protein composition comprising targeted amounts of peanut allergens.

FIG. 19 shows the native gels of purified standards of nAra h 3 (lanes 2(4 ug) and 3(2 ug)) and nAra h 1(lanes 4 (4 ug) and 5 (2 ug)) and crude extract (filtered) (lanes 6 (30 ug) and 7 (15 ug)) purified crude extract (lane 8 (15 ug)) and comparator extract (lane 9 (15 ug). Molecular size indicators (lanes 1 and 10).

FIG. 20 shows the protein bands eluting on native electrophoresis gel from the following samples: A) Standard size marker; B) crude extract (filtered); C) standard size marker; D) placebo solid dosage form; E) crude extract (filtered) added to placebo; F) Crude extract (filtered); and G) solid dosage form formulated with purified extract.

### DETAILED DISCLOSURE OF THE INVENTION

### Definitions

As used herein, the term “peanut” is interchangeable with the term's “groundnut” and “Arachis hypogea”.

An “allergen” refers to any substance that can induce or stimulate an IgE-mediated immune response in the body upon their repeated exposure to an individual. Typically, an allergen can bind specific IgE-antibodies raised upon the repeated exposure to an individual and/or induce Th2 immune reactions, such as immune reactions resulting in production/release of one more of the cytokines IL-4, IL-5, IL-10, and IL-13.

The term“peanut protein” is meant to designate proteins present in peanut kernels. A subfraction of peanut proteins are reported as peanut allergens.

The term “peanut allergen” is meant to denote any peanut allergen reported by the World Health Organization and International Union of Immunological Societies (WHO/IUIS) Allergen Nomenclature Sub-committee, which can be found on the web url: http://allergen.org/. An allergen would typically exist in a number of isoforms which have high amino acid sequence alignment. According to the WHO/IUIS, there are identified over 17 different peanut allergens including Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16 and Ara h 17. GenBank Accession Numbers for the cDNA sequences of exemplary allergens include L34402.1 (Ara h 1), AY007229.1 (Ara h2.0101), AY158467.1 (Ara h2.0201), AF093541.1 (Ara h 3.0101), AF086821.1 (Ara h 3.0201), AF059616 (Ara h 5), AF092846.1 (Ara h 6), AF091737.1 (Ara h 7), EU046325.1 (Ara h 7.0201), AY328088.1 (Ara h 8.0101), EF436550.1 (Ara h 8.0201), EU159429.1 (Ara h 9.0101), and EU161278.1 (Ara h 9.0201), AY722694.2 (Ara h 1 0.0101), AY722695.1 (Ara h 10.0201), DQ097716.1 (Ara h 11), EY396089.1 (Ara h 12), EY396019.1 (Ara h 13), AAK13449 (Ara h 14.0101), AAK13450 (Ara h 14.0102), AAT11925 (Ara h 14.0103), AAU21501 (Ara h 15.0101), respectively.

The term “Ara h 1” designates peanut allergen species with the biochemical name Cupin (Vicillin-type, 7S globulin) having a molecular weight about 64 kDa, which exists in different isoforms, for example Ara h 1.0101 having the amino acid sequence of UniProt protein P43238.

The term “Ara h 2” designates peanut allergen species with the biochemical name Conglutin (2S albumin) having a molecular weight about 17 kDa, which exists in different isoforms, for example Ara h 2.0201 having the amino acid sequence of UniProt protein Q6PSU2-1.

The term “Ara h 3” designates peanut allergen species with the biochemical name Cupin (Legumin-type, 11S globulin, Glycinin) having a molecular weight about 60 kDa (or a fragment thereof (37 kDa)), which exists in different isoforms, for example Ara h 3.0101 having the amino acid sequence of UniProt protein 082580 or Ara h 3.0202 having the amino acid sequence of UniProt protein Q9SQH7.

The term “Ara h 6” designates peanut allergen species with the biochemical name Conglutin (2S albumin) having a molecular weight about 15 kDa, which exists in different isoforms, for example Ara h 6.0101 having the amino acid sequence of UniProt protein Q647G9.

Thus, the four key allergen species disclosed herein (Ara h 1, 2, 3 and 6) may be represented by their isoforms described above. Furthermore, the four allergen species disclosed herein is meant to be represented by Ara h 1, Ara h 2, Ara h 3 and Ara h 6 species obtained by aqueous extraction of raw peanut kernels, for example of raw peanut kernels of the cultivar variant Runner (alternatively of the cultivar Virginia, Spanish and Valencia) and by using multistep purification to obtain separate pure fractions of nAra h 1, nAra h 2, nAra h 3 and nAra h 6. The production and characterization of the four key allergens are further described in Example under the heading: pure reference standards of nAra h 1, nAra h 2, nAra h 3 and nAra h 6.

Thus, all peanut allergens exist in different isoforms (isoallergens), which can be detected by sequence alignment with the above-mentioned specific isoforms reported by (WHO/IUIS) and by identification of unique amino acid fragments shared among different isoforms of each of the allergen species. Numerous isoforms for peanut allergens and unique amino acid fragments are described in patent application WO2017115139.

The prefix “n” before the name of each of the allergens Ara h 1, Ara h 2, Ara h 3 and Ara h 6 (i.e. nAra h 1, nAra h 2, nAra h 3 and nAra h 6) is meant to designate that the allergens are present in various natural occurring variants or isoforms, including various natural occurring post-translational derivatisation (e.g. glycosylation). According to Breiteneder and Chapman (2014), natural allergens may be denoted by the prefix (n) to distinguish them from recombinant allergens, which are indicated by the prefix (r) before the allergen name (e.g., nBet v 1 versus rBet v 1) and the term “natural allergen” should be used to indicate any allergen purified from a natural source material. Thus, the allergens with the prefix “n” do not include recombinantly produced allergens, or degraded allergens (e.g., allergoids). The term “native allergens” has implications for the protein structure and merely refers to the native conformation of the allergens. For example, it might be important to maintain the same oligomerisation level of the natural peanut allergens (i.e., trimer for Ara h 1, trimer+hexamer for Ara h 3, and monomer for each of Ara h 2 and Ara h 6). Natural allergens are typically obtained by gentle extraction of allergen source materials with aqueous solvents, optionally buffered aqueous solvents, and may further be subject to centrifugation or filtering. To the extent that it is understood that the key allergens are meant not to be recombinantly produced or otherwise not represent natural conformations and isoforms of the allergen species, the prefix “n” might be omitted.

The term “extractable” designates “matter” having the ability to be extracted from a source material of said matter into aqueous solvent in solubilized form. In contrast to the source material, the aqueous solution would contain an enrichment for the matter being able to be released and dissolved in the presence of an aqueous solvent. More specifically, the term “extractable” would in the present context designate the peanut proteins that are able to be extracted from peanut kernels in solubilised form, for example by soaking peanut kernels (preferably pulverised, defatted raw peanut kernels) in aqueous solvent. The resulting aqueous solvent (may now be termed peanut allergen extract) would contain an enriched for water-soluble peanut proteins. This portion of peanut proteins would reflect the peanut proteins released in saliva or in the gastro-intestinal tract following intake of peanut. In the present context, the solvent used for extraction may be an aqueous solvent, such as pure water, optionally buffered and including salts to create similar pH and tonicity conditions comparable to that in saliva. The aqueous extraction solvent might preferably not contain organic solvents, optionally it might contain sporadic amounts of organic solvents, such as ethanol, methanol, acetonitrile or the like. Peanut proteins that are extractable from peanuts with an aqueous solvent encompasses at least each of the allergens nAra h 1, nAra h 2, nAra h 3, and nAra h 6 and, optionally, further peanut allergens.

In the present context, the term “controlled” in association with compositions comprising each of the allergens nAra h 1, nAra h 2, nAra h 3, and nAra h 6, is meant to designate that these allergens are present in the composition in pre-selected concentrations different from those resulting from soaking pulverised defatted raw peanuts in an aqueous solvent. The controlled concentrations would for comparable reasons between liquid and solid composition be expressed relatively to one or more of said allergens, for example Ara h 2. In preferred embodiments, the pre-selected concentrations are controlled by a suitable quantitative analytical method as described herein to confirm the compliance with the pre-selected concentrations.

The term “controllable” is meant to designate that the concentration of one or more of the allergens selected from nAra h 1, 2, 3 and 6 can be controlled in the compositions described herein with sufficiently specificity and accuracy without interference from other constituent in the compositions.

The phrases “high molecular weight complexes” or “high mass aggregates” may be used interchangeable in the sense that in the present context both phrases is meant to designate the association of individual compounds, allergens, proteins into aggregates/complexes, which are not necessarily hold together by covalent binding, but hold together in a format detected as individual peaks when subjecting the aggregates/complexes to size exclusion chromatography. The complexes and aggregates are considered to have a high molecular weight or mass if the mass is above 500 kDa, such as above 700 kDa.

The expression “analytical scale” implies that the method in question is applicable to “small” samples of the tested material, and that the analysis can be run with high accuracy and precision.

The expression “preparatory scale” implies that the method in question is applicable to “larger” sample sizes, such as industrial scale and that the method allows for being used in the generation of output in large quanta and which may be collected or analysed by another analytical tool.

The expression “about” used in association with the specification of daily doses of peanut protein or nAra h 2 is meant to express a certain variation/tolerance around the intended daily dose of peanut protein or nAra h 2. Compositions disclosed herein are produced and analysed by methods having a high grade of complexity, for which reason the concentration of allergens and peanut protein in the compositions may be detected with high variation/low tolerance around the theoretical or nominal concentration. Therefore, any daily dose of peanut protein or nAra h 2 in the range from 0.1 μg to 99 μg peanut protein has a tolerance of ±20%, any daily dose of peanut protein or nAra h 2 from 100 to 999 μg has a tolerance of ±15% and any daily dose of peanut protein or nAra h 2 from 1000 to 9999 μg has a tolerance of ±10%. By example, a daily dose of about 1 μg peanut protein is meant to express that the daily dose of peanut protein ranges from 0.8 to 1.2 μg peanut protein, a daily dose of about 40 μg peanut protein is meant to express that the daily dose of peanut protein ranges from 32 to 48 μg peanut protein, a daily dose of about 120 μg peanut protein is meant to express that the daily dose of peanut protein ranges from 102 to 138 μg peanut protein, and a daily dose of about 1020 μg peanut protein is meant to express that the daily dose of peanut protein ranges from 918 to 1122 μg peanut protein.

Likewise, the expression “about” used in association with the specification of daily doses of the total amount of peanut proteins per unit dose or the amount of nAra h 2 per unit dose is meant to express a certain variation/tolerance around the intended unit dose of peanut protein or nAra h 2. The tolerance is similar with those specified for the daily doses defined supra.

The expression “pharmaceutical composition” when used in the present description and claims describes a composition of matter, which includes the four nAra h species 1, 2, 3, and 6 (as defined herein with respect to structure and relative/total amounts and purity) in admixture with any pharmaceutically acceptable carrier(s), vehicle(s), excipient(s), diluent(s) so that an amount of the nAra h species-containing composition can be administered via a selected route of administration to an individual, where the amount is immunologically effective and pharmaceutically/medically acceptable. It might be understood that the pharmaceutical composition is suitable for being administered to a individual in need thereof and in a quality acceptable for a regulatory medicinal agency like U.S. Food and Drug Administration (FDA) or European Medicines Agency (EMA).

### SPECIFIC EMBODIMENTS OF THE INVENTION

### Embodiments of the 1^{st} Aspect of the Invention

A first aspect relates to a composition comprising each of the peanut proteins nAra h 1, nAra h 2, nAra h 3, and nAra h 6, and may be characterised by several different options individually or combined, such as by one or more features a) to k), wherein the features are as follows:
a) being essentially free from peanut protein having a molecular mass of ≥700 kDa. The molecular mass may be determined by analytical scale size exclusion HPLC; and/or
b) an aqueous sample of the composition can be loaded onto a reverse phase HPLC column and eluted to separate nAra h 1, nAra h 2, nAra h 3, and nAra h 6 into quantifiable polypeptides when the reverse phase HPLC column is subjected to elution by mixed isocratic and gradient elution, which comprises mixing an eluent A consisting of aqueous 0.1% trifluoroacetic acid with increasing volumes of an eluent B consisting of acetonitrile mixed with 0.1% trifluoroacetic acid; and/or
c) being essentially free from peanut protein, which due to molecular size constraints is incapable of being loaded and/or separated in a reverse phase HPLC column. That is to say that nAra h1, nAra h 2, n Ara h 3, and nAra h 6 preferably are in non-denatured conformations; and/or
d) being essentially free from high molecular weight complexes of peanut-derived protein, where said high molecular complexes are characterised by being present in a discarded fraction, which can be obtained by extraction of peanut protein from raw peanut kernels (preferably pulverized raw peanut kernels) with an aqueous solvent to obtain an aqueous extract comprising each of of nAra h 1, nAra h 2, nAra h 3, and nAra h 6, subsequently subjecting the aqueous extract of extracted peanut proteins to preparative scale anion exchange chromatography with stepwise or continuous aqueous salt gradient elution at a pH in the range of 7 to 9, whereby each of nAra h 1, nAra h 2, nAra h 3, and nAra h 6 are eluted, wherein the gradient elution is continued with higher salt concentrations after elution of each of nAra h 1, nAra h 2, nAra h 3, and nAra h 6, whereby said discarded fraction is a fraction eluted after nAra h 1, nAra h 2, nAra h 3, and nAra h 6 or is retained by the anion exchange chromatography; and/or
e) comprising a controlled concentration of one or more of nAra h 1, nAra h 2, nAra h 3 and/or nAra h 6; and/or
f) comprising a controlled concentration of each of nAra h 1, nAra h 2, nAra h 3, nAra h 6; and/or
g) being obtained or obtainable by a process comprising the steps of i) extracting peanut protein from raw peanut kernels, preferably from pulverized raw peanut kernels, with an aqueous solvent to obtain an aqueous extract comprising each of of nAra h 1, nAra h 2, nAra h 3, and nAra h 6 and ii) purifying the extracted proteins by anion exchange chromatography, said chromatography comprises loading said aqueous extract to an anion exchange material and eluting with a salt gradient elution to collect fractions individually enriched for nAra h 1, nAra h 2, nAra h 3, or nAra h 6 and iii) combine fractions or aliquots thereof individually enriched with one or more of nAra h 1, nAra h 2, nAra h 3, and nAra h 6 and preferably to discard fractions eluting later than the enriched fractions of nAra h 1, nAra h 2, nAra h 3, and nAra h 6; and/or
h) being obtained or obtainable by a process comprising the steps of:
   1) extracting peanut protein from raw peanut kernels, preferably from pulverized raw peanut kernels, with an aqueous solvent to obtain an aqueous extract comprising each of of nAra h 1, nAra h 2, nAra h 3, and nAra h 6; and
   2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 is eluted and collected into one or more individual fractions; and
   3) optionally collecting a flow-through fraction from the anion exchange chromatography; and
   4) optionally continuing the stepwise or continuous salt gradient after the elution of each of nAra h 1, nAra h 2, nAra h 3, and nAra h 6 to obtain a fraction to be discarded; and
   5) combining fractions or aliquots thereof obtained in step 2, optionally obtained in both step 2 and step 3, to obtain said composition; and/or
i) being obtained or obtainable by a process comprising the steps of:
   1) extracting peanut protein from raw peanut kernels, preferably from pulverized raw peanut kernels) with an aqueous solvent to obtain an aqueous extract comprising each of of nAra h 1, nAra h 2, nAra h 3, and nAra h 6; and
   2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 is eluted and collected into fractions individually enriched for nAra h 1, nAra h 2, nAra h 3, or nAra h 6;
   3) optionally collecting a flow-through fraction from the anion exchange chromatography; and
   4) combining fractions or aliquots thereof obtained in step 2, optionally obtained in both step 2 and step 3, to obtain said composition; and/or
j) comprising molar ratios nAra h 1:nAra h 2, nAra h 3:nAra h 2, nAra h 6:nAra h 2, which each are in the range between 0.5 and 1.5. The concentration of each of the allergens may be quantified by analytical scale RP-HPLC and/or LC-MS/MS. For converting mass units to molar concentrations, the following molar masses may be used: 68757 g/mol for Ara h 1, the molar mass of 17994 g/mol for Ara h 2, the molar mass of 58600 g/mol for Ara h 3 and the molar mass of 14846 g/mol for Ara h 6; and/or
k) being enriched for total amount of water-soluble peanut proteins selected from nAra h 1, nAra h 2, nAra h 3, and nAra h 6 per weight unit total peanut protein compared to an aqueous extract of pulverised raw peanut kernels that have been subjected to extraction with an aqueous solvent.

In a preferred aspect, the compositions comprises a molar ratio of each of the pairs nAra h 1:nAra h 2, nAra h 3:nAra h 2 and nAra h 6:nAra h 2 is in the range of 0.5 to 2.0, such as in the range of 0.5 to 1.5. Such compositions may further comprise a pharmaceutically acceptable carrier, diluent, excipient, or vehicle to form a pharmaceutical composition, which may be suitable for treating peanut allergy.

The composition of option a) is primarily characterised by not including high molecular mass proteins. Such proteins may be large aggregates composed of one or more of the four allergens nAra h 1, nAra h 2, nAra h 3, and nAra h 6. In particularly, the present inventors found that nAra h 1 and nAra h 3 polypeptides forms part of such high molecular mass aggregates (or oligomers/polymers) in aqueous extracts obtained from raw peanut kernels and they may cause precipitation issues. The presence of such high molecular mass aggregates incurs difficulties in the proper managing and quantification of the amount of nAra h 1 and/or nAra h 3 in peanut protein composition and will not be water-soluble to an extent that will prevent precipitation issues.

The high molecular mass proteins and their size may be detected by various analytical tools, preferably by analytical scale size exclusion HPLC. Preferably, the analytical scale size exclusion HPLC of option a) and of other embodiments disclosed herein may be capable of separating the five size-indicating reference standards thyroglobulin (having a molecular mass of 670 kDa), bovine γ-globulin (having a molecular mass of 158 kDa), chicken ovalbumin (having a molecular mass of 44 kDa), equine myoglobin (having a molecular mass of 17 kDa), and vitamin B12 (having a molecular mass of 1.35 kDa) by elution with aqueous (for instance phosphate) buffered saline having pH in the range of 7 to 7.5. Other size indicating markers may be used to further differentiate between sizes above 700 kDa. The size-indicating references are eluted in the order of decreasing mass. The size exclusion HPLC may further comprise the detection of peaks with UV absorbance at 210 nm or 280 nm.

It will be evident that peanut compositions do not contain essential amounts or only sporadic amounts of high molecular mass proteins above size 700 kDa if the resulting chromatogram is essentially free from peanut protein peaks eluting with a mass similar to or higher than the size-indicating reference standard thyroglobulin which has a molecular mass of 670 kDa. The chromatograms may eventually comprise other peaks eluting with a mass similar with, or higher than the size-indicating reference standard thyroglobulin, which peaks would derive from a non-peanut protein source present in the sample.

In further important embodiments, the composition of the 1^{st} aspect of the invention is being essentially free from peanut protein (such as in the form of aggregates) having a molecular mass of >650 kDa, preferably >600 kDa, such as >550 kDa, >500 kDa, >450 kDa, >450 kDa, >400 kDa, said molecular mass being determined by analytical scale size exclusion HPLC.

In the present context, “essentially free” means that a peanut protein above the indicated molecular mass threshold does not provide a visible signal/peak in the size exclusion chromatogram determined by UV absorbance at 210 nm, which goes beyond a threshold of 1% of the total peak area in the chromatogram, more preferably beyond a threshold of 0.8%, such as 0.7%, such as 0.6% of the total peak area in the chromatogram. When determining the total peak area, the peaks eluting at or before the void volume (e.g. the buffer peaks) should not be included in the total peak area. In some embodiments, the total peak area may be determined by summarising the peak areas of peaks eluting between the void volume and the reference standard equine myoglobin (having a molecular mass of 17 kDa) or vitamin B12 (having a molecular mass of 1.35 kDa).

The peanut proteins in the compositions, and thus each of nAra h 1, nAra 2, nAra h 3 and nAra h 6, are characterised by being extractable from peanut kernels by an aqueous solvent. Thus, the four allergens may be extracted from raw peanut kernels by an aqueous solvent to obtain the allergens in water-soluble form and in their natural conformation. Thus, it is preferred that the peanut kernels have not been subjected to any processing which could alter the natural primary sequence or conformation of a peanut protein, which otherwise will result from roasting, heating, or blanching, which causes denaturation, degradation, or aggregation. Preferably, the raw peanut kernels are pulverised, and may optionally be skinned before pulverisation. The aqueous extraction solvent might preferably not contain any organic solvents, such a as alcohols (methanol, ethanol, propanol, butanol) or acetonitrile or the like. Such organic solvents might cause denaturation of the proteins. Optionally the solvent might contain sporadic amounts of organic solvents. Peanut proteins that are extractable from peanuts with an aqueous solvent encompasses at least each of the allergens nAra h 1, nAra h 2, nAra h 3, and nAra h 6 and, optionally, further peanut allergens.

In some embodiments, the extraction of peanut proteins from raw peanut kernels with an aqueous solvent in embodiments herein, in particularly option d), g), step 1 of option h) or step 1 of option i) comprises extraction with a buffered aqueous solvent having a pH in the range of 6 to 9, optionally a buffered saline aqueous solvent having pH in the range of 6 to 9.

Due to the poor extraction efficiency of nAra h 3 at pH values below neutral pH, the buffered, optionally saline aqueous solvent may have a pH in the range of 6.5 to 9, such as in the range of 6.5 to 8.5, such as in the range of 6.5 to 8.5. In even more preferred embodiments, the pH is in the range of 7 to 9, such as 7 to 8.5, such as in the range of 7 to 8. The pH may be kept under 9, such as below 8.5 to avoid precipitation of nAra h 3 when the extracted solution is allowed to stand before being used in the following step. The buffer can be or may comprise any buffering agent suitable for buffering in the indicated pH range, for example phosphate buffer, imidazole buffer or TRIS (tris(hydroxymethyl)-aminomethane buffer). By example, the buffered aqueous solvent can be or may comprise TRIS in a molar range of 10 to 200 mM, preferably in the range of 10 to 100 mM, such as in the range of 10 to 50 mM. The buffer optionally comprises saline which may be NaCl or an equivalent salt thereof in an amount in the range of 5 to 200 mM, preferably in the range of 10 to 100 mM, 10 to 50 mM. In this context, the term “equivalent salt” is intended to include fully dissolvable salts such as NaI, KCl, KI, NH₄CI, NH₄I, MgCl₂, MgI₂, Na₂SO₄, K₂SO₄, or NH₄SO₄, which do not interact with any of the four allergens in a manner, which will impede their immunologic activity or solubility. Typically, the aqueous solvent can be composed of 50 mM TRIS and 50 mM NaCl in purified water, pH adjusted to 7.4 with 2.0 M NaOH.

The stepwise or continuous aqueous salt gradient elution used in the processes described herein and in particularly in respect of option d), g), step 2 of option h) or step 2 of option i) may be carried out at a pH in the range of 6 to 9, such as in the range of 6.5 to 9, such as 6.5 to 8.5. To obtain better adsorption/retention of the peanut proteins on the anion exchange, the pH may be above 7, such as preferably in the range of 7 to 8.5, such as preferably in the range of 7 to 8, such as in the range of 7.2 to 7.8. Also, the stepwise or continuous aqueous salt gradient elution in option d), g), step 2 of option h) or step 2 of option i) is preferably carried out using NaCl as the salt or using a salt equivalent to NaCl.

The peanut kernels used in embodiments herein may be of cultivated peanuts (Arachis hypogaea) come in many botanical varieties, but there are four basic types: Runner, Virginia, Spanish and Valencia. In compositions of the 1^{st} aspect, the raw peanut kernels are preferably from the cultivar named Runner, since this cultivar variety seems to comprise the highest number of allergen isoforms. As mentioned, the peanut kernels are raw peanut kernels, and may either be skinned or unskinned. Most importantly, the peanut kernels might not have been processed by heating, boiling or roasting if this causes denaturation, degradation ed allergens and aggregation.

Several cultivar types of peanuts exist, but typically the peanuts are of the cultivar type selected from the group consisting of Runner, Virginia, Spanish and Valencia. It has been found that peanut cultivar of the type of Runner may contain many allergen isoforms, in particularly of the genotype named #1041.

Before extraction, the peanut kernels are preferably defatted and pulverised and optionally skinned before defatting and pulverisation. By example, the peanut kernels are defatted by conventional known chemically or mechanically defatting methods. By example, the defatting can be done by use of a mechanical oil press to produce crushed defatted peanut material, which optionally is pulverised by a blender or grinder. The defatted peanut kernels typically comprise up to 15% by weight of oil, preferably in the range of 5 to 12% by weight of oil.

The compositions may contain a pre-selected and/or controlled concentration of one or more of the four peanut allergens n Ara h 1, 2, 3, and/or 6. The allergen nAra h 3 is a complex allergen naturally occurring in a numerous of isoforms and in oligomeric conformations. In compositions disclosed herein, nAra h 3 was found to exist as a monomer comprising one polypeptide sequence of nAra h 3, a trimer comprising three polypeptide sequences of nAra 3 and a hexamer comprising six polypeptide sequences of nAra h 3. Further, nAra h 3 polypeptides form parts of the high molecular mass aggregates with size above 700 kDa. The controlled concentration of nAra h 3 in embodiments disclosed herein and in particularly in option(s) e) and/or f) can be determined by means of a quantitative immune assay, an analytical scale reverse phase HPLC or a quantitative LC-MS/MS. In one embodiment, the analytical scale reverse phase HPLC method is the preferred method as it has been found that nAra h 3 can be quantified by analytical scale reverse phase HPLC to express the molar content of nAra h 3 in terms of the molar content of nAra h 3 monomer (content of single polypeptide nAra h 3). Therefore the concentration of nAra h 3 is conveniently determined by analytical scale reverse phase HPLC that are able to separate each of the four key peanut allergens, for example comprising separation of nAra h 1, nAra h 2, nAra h 3, and nAra h 6 by use of mixed isocratic and gradient elution, which gradient elution comprises mixing an eluent A consisting of aqueous 0.1% trifluoroacetic acid with increasing volumes of an eluent B consisting of acetonitrile with 0.1% trifluoroacetic acid. The quantification may be performed against a pure calibration standard of nAra h 3, and optionally converting the concentration by weight of nAra h 3 in the composition to molar concentration of nAra h 3 by using a molar mass of 58,600 g/mol for Ara h 3. The RP-HPLC may further comprise the detection of peaks with UV absorbance at 210 nm or 280 nm, or peaks may be detected by mass spectrometry.

The compositions disclosed herein may be characterised by having a concentration of at least nAra h 2, and/or one or more of the other three key allergens (e.g. nAra h 3) in controlled concentrations or the concentrations allow for being controllable. Thus, compositions may comprise controlled or controllable concentrations of each of nAra h 1, nAra h 2, nAra h 3 and nAra h 6.

The concentration of nAra h 3 in compositions disclosed herein and in particularly of option(s) e) and/or f) is preferably in the range of 12% to 70% % by weight of the total mass of peanut proteins in the composition. nAra h 3 has a high molecular mass and may constitute more than 50% by weight of the peanut proteins, for example such from 12% to 60%, such as from 15% to 60%, such as from 20% to 60%, such as from 25% to 55%, such as from 17% to 53%, such as from 15% to 50% by weight of the total mass of peanut proteins in the composition. More narrow percentage ranges are envisaged, such as 18%-46%, and in particularly 21%-42% by weight of the total mass of peanut proteins. In the context of molar concentration, the molar concentration of nAra h 3 in embodiments disclosed herein, in particular of option(s) e) and/or f) is preferably in the range of 2-12 nmol/mg peanut protein, such as in the range of 3-11 nmol/mg, such as in the range of 3-10 nmol/mg, such as in the range of 3-9 nmol/mg, such as in the range of 4-8 nmol/mg such as in the range of 2.8 to 8.4 nmol/mg of the total mass of peanut proteins, preferably in the range of 3.1 to 7.8 nmol/mg, such as in the range of 3.6 to 7.1 nmol/mg of the total mass of peanut proteins.

While nAra h 2 is considered the critical peanut allergen in association with anaphylaxis, the compositions disclosed herein may at least contain a pre-selected or controlled concentration of nAra h 2. Preferred concentrations of nAra h 2 may be from 4% to 20% by weight of the total mass of peanut proteins in the composition, such as from 4% to 18%, such as from 5% to 15%. More narrow concentrations may be considered such as from 5.5% to 14%, such as from 6.5% to 13%, such as from 7% to 12% by weight of the total mass of peanut proteins in the composition. In the context of molar concentration, the molar concentration of nAra h 2 in embodiments disclosed herein is preferably is preferably in the range of 2-12 nmol/mg peanut protein, such as in the range of 3-11 nmol/mg, such as in the range of 3-10 nmol/mg, such as in the range of 3-9 nmol/mg, such as in the range of 4-8 nmol/mg such as in the range of 2.8 to 8.4 nmol/mg of the total mass of peanut proteins, preferably in the range of 3.1 to 7.8 nmol/mg, such as in the range of 3.6 to 7.1 nmol/mg of the total mass of peanut proteins.

With a carefully controlled concentration of nAra h 2 in the compositions, the concentration of other peanut allergens may be adjusted to be within a molar ratio to the molar concentration of nAra h 2, such as in the range of 0.5 to 2.0 (50% to 200% of the concentration of nAra h 2) or more preferably, the molar ratio is in the range 0.5 to 1.5 (50% to 150% of the concentration of nAra h 2). Such compositions are considered to provide substantially equimolar doses of each of the four key allergens when administered to a subject in need thereof. The target range for equi-molarity may be even more narrow, such as in the range of 0.6 to 1.4 (60% to 140%) or in the range of 0.7-1.3 (70 to 130%) but might be difficult to obtain due to variability in peanut source material, production method and analytical methods used for quantification.

The concentration of each of Ara h 1, nAra h 2, nAra h 3 and nAra h 6 in embodiments herein and in particularly in option f) is preferably determined by means of a quantitative immune assay, analytical scale reverse phase HPLC or quantitative LC-MS/MS. In particular, the concentration of nAra h 1, nAra h 2, nAra h 3 and nAra h 6 can be determined by analytical scale reverse phase HPLC comprising separation of nAra h 1, nAra h 2, nAra h 3, and nAra h 6 by use of mixed isocratric and gradient elution, which gradient elution comprises mixing an eluent A consisting of aqueous 0.1% trifluoroacetic acid with increasing volumes of an eluent B consisting of acetonitrile with 0.1% trifluoroacetic acid. Quantification may include quantification against pure calibration standards of nAra h 1, nAra h 2, nAra h 3, and nAra h 6 and the concentration may optionally be converted to molar concentrations of nAra h 1, nAra h 2, nAra h 3 and nAra h 6 by weight in the composition by using the molar mass of 68,757 g/mol for Ara h 1, the molar mass of 17,994 g/mol for Ara h 2, the molar mass of 58,600 g/mol for Ara h 3 and the molar mass of 14,846 g/mol for Ara h 6.

The concentration of nAra h 1, nAra h 2, nAra h 3 and nAra h 6 may also be determined by use of LC-MS/MS with MS quantification using heavy signature peptides (e.g., AQUA peptides) to each of the four allergens. By this MS method, the allergens of the composition of the 1^{st} aspect or an aqueous dissolution of said composition is digested by treatment with digestive enzymes like trypsin or chymotrypsin. Known concentrations of synthetic isotope labelled signature peptides are then added to the protein digest of the extract. According to this method, the presence of high molecular mass aggregates of the allergens in the composition would also be digested and then be quantified as well. Thus, where the composition comprises high molecular mass aggregates of nAra h 1 or nAra h 3, this MS method would fail to quantify the concentration of nAra h 1 or nAra h 3 that is in the natural conformation and water-soluble. As shown by the present inventors, the four allergens of the compositions of the 1^{st} aspect of the invention which are substantially free of high molecular mass aggregates can be quantified equally well by use of the RP-HPLC with UV detection and by the LC-MS/MS method as there was good correlation between the concentration of each of the allergens obtained by the two methods.

The concentration by weight of the total mass of peanut proteins in the composition of each of nAra h 1, nAra h 2, nAra h 3 and nAra h 6 in compositions disclosed herein and in particularly of option f) is typically in the range of 20% to 60% for nAra h 1, in the range of 5% to 15% for nAra h2 (optionally 4% to 20%), in the range of 15% to 50%((optionally in the range of 20 to 60%) for nAra h 3, in the range of 4% to 12% optionally in the range of 4-18%) for nAra h 6, provided that the sum of nAra h 1, nAra h 2, nAra 3 and nAra 6 at least constitute 75% by weight of total peanut protein. Typically, the concentration by weight of the total mass of peanut proteins for nAra h 1 is in the range of 20% to 60%; for nAra h 2 it is in the range of 4% to 20%; for nAra h3 it is in the range of 20% to 60% and for nAra h 6 it is in the range of 4% to 18%, provided that the sum of nAra h 1, nAra h 2, nAra 3 and nAra 6 constitute at least 75% by weight of total peanut protein, or typically the concentration by weight of the total mass of peanut proteins for nAra h 1 is in the range of 25% to 60%; for nAra h2 it is in the range of 6% to 14%; for nAra h3 it is in the range of 20% to 55% and for nAra h 6 it is the range of 5% to 15%, provided that the sum of nAra h 1, nAra h 2, nAra 3 and nAra 6 at least constitute 75% by weight of total peanut protein.

Preferably, the concentration by weight of the total mass of peanut proteins in the composition of each of nAra h 1, nAra h 2, nAra h 3 and nAra h 6 in option f) is in the range of 21% to 53% for nAra h 1, in the range of 5.5% to 14% for nAra h2, in the range of 18% to 46% for nAra h 3, in the range of 5% to 11% for nAra h 6, provided that the sum of nAra h 1, nAra h 2, nAra 3 and nAra 6, provided that the sum of nAra h 1, nAra h 2, nAra 3 and nAra 6 at least constitute 75% by weight of total peanut protein. Even more preferred are embodiments where the controlled concentration by weight of the total mass of peanut proteins in the composition of each of nAra h 1, nAra h 2, nAra h 3 and nAra h 6 in option f) is in the range of 25% to 50% for nAra h 1, in the range of 6.5% to 13% for nAra h2, in the range of 21% to 42% for nAra h 3, in the range of 5% to 11% for nAra h 6, provided that the sum of nAra h 1, nAra h 2, nAra 3 and nAra 6 at least constitute 75% by weight of total peanut protein.

At any rate, the total concentration (the sum) of nAra h 1, nAra h 2, nAra 3 and nAra 6 normally constitute at least 75% by weight of the total peanut protein in the composition, such as at least 80%, such as at least 85%, such as at least 90% by weight of the peanut proteins, and the sum of the combined concentrations constitute at the most 98%, 99% or 100% by weight of the total peanut protein. Where the compositions comprise additional allergens, the four key allergens typically constitute from 75% to 99% by weight of total peanut protein, preferably from 75% to 98%, such as from 80% to 98%, 85% to 98% or more narrow. Where it is desirable to provide compositions only including the four key allergens, the four allergens may constitute about 100% by weight of the peanut proteins in the compositions, such as between 99 to 100%. In the context of molar ratios, the concentration of each of nAra h 1, nAra h 2, nAra h 3 and nAra h 6 in compositions disclosed herein, and particularly of option f) is in the range of 2-12 nmol/mg of the total mass of peanut proteins in the composition, such as in the range of 3-11 nmol/mg, such as in the range of 3-10 nmol/mg, such as in the range of 3-9 nmol/mg, such as in the range of 4-11 nmol/mg such as in the range of 4-10, such as 4-9 nmol/mg. In typical embodiments, the concentration of the four key allergens is in the range of 2.8 nmol/mg to 8.4 nmol/mg of the total mass of peanut proteins in the composition, more preferably in the range of 3.1 to 7.8 nmol/mg, such as in the range of 3.6 to 7.1 nmol/mg of the total mass of peanut proteins in the composition. The relative concentrations between the four key allergens may be carefully pre-selected or controlled to comply with a molar ratio of each of the pairs nAra h 1:nAra h 2, nAra h 3:nAra h 2, nAra h 6:nAra h 2 in the range of 0.5 to 2.0, such as in the range of 0.5 to 1.5 or more narrow, such as in the range of 0.6 to 1.4 and preferably in the range 0.7 to 1.3. Other ranges to consider are in the range of 0.4 to 1.6 or in the range 0.3 to 1.7. As mentioned, analytical methods suitable for controlling the concentrations are by analytical scale RP-HPLC or quantitative immune assay, like ELISA.

The compositions disclosed herein may not be obtained by simple extraction of peanut kernels as this type of processing would not result in balanced molar concentrations between the four allergens and will be difficult to run in a reproducible manner in industrial scale settings. The present inventors have provided a simple preparation method allowing for the design of peanut protein compositions comprising pre-selected concentrations of the four key allergens.

The compositions disclosed herein may not be obtained by simple extraction of peanut kernels as this type of processing would not result in the balanced molar concentrations (equimolar concentrations) between the four allergens and will be difficult to run in a reproducible manner in industrial scale settings. The present inventors have provided a simple preparation method allowing for the design of peanut protein compositions comprising pre-selected concentrations of the four key allergens. The key step in this process comprises the adsorption of the peanut allergens to an anion exchange chromatography material and the ability to collect individual fractions enriched for either of the allergens nAra h 1, nAra h 2, nAra h 3 and nAra h 6 following treating the anion exchange material with different salt concentrations. The enriched fractions can be mixed to obtain pre-selected concentrations of two or more of the four key allergens.

A suitable process for obtaining the compositions disclosed herein may be obtainable by a process comprising the steps of:
1) providing an aqueous extract of peanut protein obtained by extracting raw peanut kernels (preferably pulverized defatted peanut kernels) with an aqueous solvent to obtain an aqueous extract comprising each of of nAra h 1, nAra h 2, nAra h 3, and nAra h 6; and
2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 is eluted and collected into fractions individually enriched for nAra h 1, nAra h 2, nAra h 3, or nAra h 6; and
3) optionally collecting a flow-through fraction from the anion exchange chromatography; and
4) combining fractions or aliquots thereof as obtained in step 2 or combined step 2 and 3 to obtain said peanut proteins.

A simpler process might be feasible. For example, the extraction step to obtain the four allergens in dissolved form (for example step 1 above) may be combined with the process step (for example step 2 above) of adsorbing the allergens on the anion exchange chromatography material. Such a process may comprise soaking pulverized defatted raw peanut kernels in an aqueous solvent in the presence of anion exchange chromatography material to let the extracted allergens be adsorbed onto anion exchange material. Further step comprises removing the aqueous solvent (e.g., by filtering or decanting) from the anion exchange material. Further step comprises stepwise addition of increasing concentrations of aqueous salt solution at pH in the range of 6 to 9, wherein within each step the aqueous salt solution is removed (e.g. by filtering or centrifugation) to obtain individual fractions each enriched for nAra h 1, nAra h 2, nAra h 3, or nAra h 6. As in the above process the fractions may be combined to obtain peanut compositions comprising two or more of the allergen species nAra h 1, nAra h 2, nAra h 3, or nAra h 6.

It should be understood that in the step 2 of the process, the peanut allergens nAra h 1, 2, 3 and 6 are able to being adsorbed, such as retained, on the anion exchange material, and any anion exchange material able to retain all four key allergens when applied in an aqueous solution in the pH range of about 7 to 9, such as 7.5 to 9 will be feasible for the purpose. A strong anion exchange material is preferred, such as a quaternary anion exchange material, e.g., the one sold under the trade name HiTrap Q HP from Cytiva™. The anion exchange material is typically packed in a column when used in step 2 above, In the simpler process, the anion exchange material might advantageously be linked to inert beads of a size that permits filtering or centrifugation to separate the beads from the extraction solvent/elution solvent. An exemplary bead/resin for use in the simpler process is sold under the trade name Chromalite MQ/C from Pyrolite.

The aqueous solvent of step 1 comprises preferably a buffered aqueous solvent having pH in the range of 7 to 9, and the salt of step 2 may be NaCl or a salt equivalent to NaCl. The pH is preferably consistent with the pH preferred for step 2, which require a pH above 7 to ensure that the proteins are negatively charged to be adsorbed on the positively charged anion exchange material.

It is preferred that the concentrations of nAra h 1, nAra h 2, nAra h 3, and nAra h 6 in the individual fractions obtained in step 2, such as in options i) h) and/or i) are determined, preferably by analytical RP-HPLC (cf. the examples for details on a preferred RP-HPLC quantification process). The fractions obtained in step 2 are eluted in the following order: nAra h 6, nAra h 2, nAra h 1 and nAra h 3.

The entire fractions or aliquots thereof which are combined in step 4 above, and in particularly in step 5 of option h) and/or in step 4 of option i) are in important embodiments combined to produce a molar ratio of each of the pairs nAra h 1:nAra h 2, nAra h 3:nAra h 2, nAra h 6:nAra h 2 in the range of 0.5 to 2.0, such as in the range of 0.5 to 1.5 or other ranges discloses herein.

In all embodiments of the 1^{st} aspect of the invention disclosed above, it is preferred that nAra h 1, nAra h 2, nAra h 3, and nAra h 6 comprise their naturally occurring isoforms and oligomeric forms. In other words, no artificially engineered mutants are included and conversely, it is important to avoid that naturally occurring isoforms are removed from the composition (when viewed relative to the peanut source material from which the composition has been derived). Thus, the compositions do not contain recombinantly expressed nAra h 1, nAra h 2, nAra h 3, and nAra h 6. The compositions rather comprise peanut proteins extractable from raw peanuts by an aqueous solvent to form an aqueous extract comprising each of the proteins nAra h 1, nAra h 2, nAra h 3, and nAra h 6.

The allergen nAra h 3 in the composition is preferably present in a conformation selected from the group consisting of monomeric nAra h 3, trimeric nAra h 3 and hexameric nAra h 3, such as in a mixture of monomeric, trimeric and hexameric nAra h 3, such as predominantly as a mixture of trimeric and hexameric nAra h 3. Similarly, nAra h 1 may be present it its monomeric and/or trimeric form, such as predominantly present in its trimeric conformation. Large multimeric forms, on the other hand, are essentially absent.

In important and preferred embodiments, the compositions are essentially free from aggregates comprising a nAra h 3 polypeptides and/or a nAra h 1 polypeptides, wherein the aggregates have a molecular mass >700 kDa. It has turned out, that nAra h 1 and nAra h 3 polypeptides form part of high molecular mass aggregates in in extracts obtained by aqueous extraction of peanut kernels, which renders the control of the concentration of the allergen difficult in compositions intended for pharmaceutical use.

The total mass of peanut protein in the composition of the 1^{st} aspect is conveniently determined by amino acid analysis (AAA) or by the Bradford protein assay using Bovine Serum Albumin as reference standard (BCA), preferably by amino acid analysis (AAA) as this method may determine the protein content more accurately.

Embodiments of the first aspect are further described in numbered embodiments NE1 to NE37.

Since the compositions of the 1^{st} aspect normally are used in pharmaceutical formulations for treatment of allergy by allergen-specific immunotherapy, the composition typically further comprises a pharmaceutically acceptable carrier, diluent, excipient, or vehicle. More details on these further pharmaceutically acceptable substances/components are provided in the discussion infra, relating to the 2^{nd} and further aspects of the invention.

### Embodiments of the 2^{nd} Aspect of the Invention

The pharmaceutically acceptable formulation/composition of the 2^{nd} aspect of the invention normally comprises a pre-selected/controlled amount of nAra h 2, and preferably a controlled amount of each of nAra h 1, nAra h 2, nAra h 3, and nAra h 6, thus in line with the discussion above dealing with the composition of the 1^{st} aspect of the invention and the embodiments thereof.

The carrier in the pharmaceutically acceptable formulation is in important embodiments a solid carrier substance, preferably a solid carrier substance suitable for forming a sublingual solid dosage form. Administration via the sublingual mucosa of allergen compositions has over time been demonstrated to involve several advantages over other routes of administration. Not at least, this administration form is more convenient for the patient in contrast to administration by injection. The solid formulation is typically in the form of a tablet (either compressed or non-compressed), a film, a paste, or a lyophilisate (e.g., unit dose lyophilisate). In preferably embodiments, the solid dosage form is a sublingual tablet, sublingual film, or sublingual lyophilizate. In important embodiments, the solid pharmaceutically acceptable formulation is fast-dispersing when exposed to human saliva, wherein the fast-dispersing solid formulation preferably is disintegrated within 2 minutes, such as within 1.5, 1, or within 0.5 minutes following the exposure to saliva. In interesting and important embodiments, the carrier substance comprises gelatine, preferably piscine gelatine, which could disperse at the above-indicated fast rates.

Pharmaceutical compositions formulated as solid dosage forms are preferred over liquid dosage forms. As disclosed herein, solid dosage forms provide good stability of each of the four key allergens for at least 12 months at room temperature about 25° C. This allows for storing the solid pharmaceutical compositions disclosed herein at room temperature in contrast to the currently marketed OIT products or liquid skin prick test products requiring storage condition between 2° C. and 8° C.

The pharmaceutically acceptable formulation of the 2^{nd} aspect can be a unit dose form, preferably a sublingual unit dose form. In such a unit dose form, the total amount of peanut proteins per unit dose form is normally in the range 0.1-5000 μg, and here it is preferred that the amount of nAra h 2 per unit dose form is in the range from 0.01-500 μg.

Although solid formulations are preferred over liquid formulations due to stability issues, the pharmaceutical compositions may in the alternative be formulated as a liquid solution, eventually as a kit of a powdered composition and a diluent for dissolving the composition before administration. Liquid formulations often contain saline to maintain isotonicity, pH adjusters, antioxidants, preservatives. In addition, many allergen products contain glycerol to decrease the free water activity for the purpose of stabilising the allergens during storage.

In still applicable formulation, the pharmaceutical composition may be formulated as a patch for application to skin such as by epicutaneous administration. Exemplary patch formulations for epicutaneous administration of peanut allergens are described in the patent application WO2009071599A1.

As is well-known in the art, it is often a prerequisite in allergen-specific immune therapy of allergy to include a so-called updosing phase, i.e. an initial phase of the treatment where daily or other periodic doses of allergen are increased, followed by a so-called maintenance phase using constant daily or other periodic doses—where the maintenance doses normally are at the same or slightly lower level per administration period than is the case for the latest dose in the updosing phase. To support such dosage regimens, the above-discussed unit doses can include different amounts of allergen. For example, the total amount of peanut proteins per unit dose form can be about 0.1 μg, about 0.5 μg, about 1.0 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 10.5 μg, about 11 μg, about 11.5 μg, about 12 μg, about 12.5 μg, about 13 μg, about 13.5 μg, about 14 μg, about 14.5 μg, about 15 μg, about 15.5 μg, about 16 μg, about 16.5 μg, about 17 μg, about 17.5 μg, about 18 μg, about 18.5 μg, about 19 μg, about 19.5 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, about 500 μg, about 505 μg, about 510 μg, about 515 μg, about 520 μg, about 525 μg, about 530 μg, about 535 μg, about 540 μg, about 545 μg, about 550 μg, about 555 μg, about 560 μg, about 565 μg, about 570 μg, about 575 μg, about 580 μg, about 585 μg, about 590 μg, about 595 μg, about 600 μg, about 605 μg, about 610 μg, about 615 μg, about 620 μg, about 625 μg, about 630 μg, about 635 μg, about 640 μg, about 645 μg, about 650 μg, about 655 μg, about 660 μg, about 665 μg, about 670 μg, about 675 μg, about 680 μg, about 685 μg, about 690 μg, about 695 μg, about 700 μg, about 705 μg, about 710 μg, about 715 μg, about 720 μg, about 725 μg, about 730 μg, about 735 μg, about 740 μg, about 745 μg, about 750 μg, about 755 μg, about 760 μg, about 765 μg, about 770 μg, about 775 μg, about 780 μg, about 785 μg, about 790 μg, about 795 μg, about 800 μg, about 805 μg, about 810 μg, about 815 μg, about 820 μg, about 825 μg, about 830 μg, about 835 μg, about 840 μg, about 845 μg, about 850 μg, about 855 μg, about 860 μg, about 865 μg, about 870 μg, about 875 μg, about 880 μg, about 885 μg, about 890 μg, about 895 μg, about 900 μg, about 905 μg, about 910 μg, about 915 μg, about 920 μg, about 925 μg, about 930 μg, about 935 μg, about 940 μg, about 945 μg, about 950 μg, about 955 μg, about 960 μg, about 965 μg, about 970 μg, about 975 μg, about 980 μg, about 985 μg, about 990 μg, about 995 μg, about 1000 μg, about 1050 μg, about 1100 μg, about 1150 μg, about 1200 μg, about 1250 μg, about 1300 μg, about 1350 μg, about 1400 μg, about 1450 μg, about 1500 μg, about 1550 μg, about 1600 μg, about 1650 μg, about 1700 μg, about 1750 μg, about 1800 μg, about 1850 μg, about 1900 μg, about 1950 μg, about 2000 μg, about 2050 μg, about 2100 μg, about 2150 μg, about 2200 μg, about 2250 μg, about 2300 μg, about 2350 μg, about 2400 μg, about 2450 μg, about 2500 μg, about 2550 μg, about 2600 μg, about 2650 μg, about 2700 μg, about 2750 μg, about 2800 μg, about 2850 μg, about 2900 μg, about 2950 μg, about 3000 μg, about 3050 μg, about 3100 μg, about 3150 μg, about 3200 μg, about 3250 μg, about 3300 μg, about 3350 μg, about 3400 μg, about 3450 μg, about 3500 μg, about 3550 μg, about 3600 μg, about 3650 μg, about 3700 μg, about 3750 μg, about 3800 μg, about 3850 μg, about 3900 μg, about 3950 μg, about 4000 μg, about 4050 μg, about 4100 μg, about 4150 μg, about 4200 μg, about 4250 μg, about 4300 μg, about 4350 μg, about 4400 μg, about 4450 μg, about 4500 μg, about 4550 μg, about 4600 μg, about 4650 μg, about 4700 μg, about 4750 μg, about 4800 μg, about 4850 μg, about 4900 μg, about 4950 μg, or about 5000 μg.

In particular, for Ara h 2 the amount can be about 0.01 μg, about 0.05 μg, about 0.1 μg, about 0.15 μg, about 0.2 μg, about 0.25 μg, about 0.3 μg, about 0.35 μg, about 0.4 μg, about 0.45 μg, about 0.5 μg, about 0.55 μg, about 0.6 μg, about 0.65 μg, about 0.7 μg, about 0.75 μg, about 0.8 μg, about 0.85 μg, about 0.9 μg, about 0.95 μg, about 1.0 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 10.5 μg, about 11 μg, about 11.5 μg, about 12 μg, about 12.5 μg, about 13 μg, about 13.5 μg, about 14 μg, about 14.5 μg, about 15 μg, about 15.5 μg, about 16 μg, about 16.5 μg, about 17 μg, about 17.5 μg, about 18 μg, about 18.5 μg, about 19 μg, about 19.5 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, or about 500 μg per unit dose.

Generally, it is preferred that the amounts of nAra h 1, nAra h 2, nAra h 3, and nAra h 6 in the pharmaceutically acceptable formulation are discussed above in relation to the 1^{st} aspect of the invention; hence, all considerations pertaining to the composition of the 1^{st} aspect apply mutatis mutandis to the 2^{nd} aspect of the invention and the embodiments thereof. Hence, in important embodiments of the 2^{nd} aspect, the pharmaceutically acceptable formulation comprises the composition of the 1^{st} aspect of the invention and any embodiments thereof disclosed herein. Embodiments of the 2^{nd} aspect is further defined in numbered embodiments NE38 to NE51, which relates to a pharmaceutical composition comprising a composition according to any of the numbered embodiments NE1 to NE37.

### Embodiments of the 3^{rd} Aspect of the Invention

The composition or pharmaceutical formulation for the use of the 3^{rd} aspect typically involves that the allergen-specific immunotherapy comprises a plurality of administrations of the composition or formulation. The plurality of administrations is preferably a plurality of administrations separated by at least one day, and where the plurality of administration most preferably is in the form of one daily dose, such as one daily administration of a dose (for instance, a daily dose can be made in the form of a number of separate administrations within the same day, but seen from a patient point-of-view, it is far more convenient to only perform one single administration per day (or per other period, if relevant).

So, the allergen-specific immunotherapy can comprise administration of a plurality of identical daily doses of peanut protein, optionally preceded by a plurality of consecutive non-identical daily doses. Or, in other words, provided it is found safe to administer one periodical (such a daily) dose throughout the entire treatment period, no need exists for an initial scheme, where non-identical doses are administered. But the allergen-specific immunotherapy can also comprise administration of a plurality of consecutive non-identical daily doses of peanut protein, optionally preceding a plurality of identical daily doses.

At any rate, the plurality of consecutive non-identical daily doses is in some embodiments in the form of consecutive daily doses where no earlier dose is higher than a later dose—i.e., a traditional updosing scheme where each daily non-identical dose is higher than any preceding dose in the series is part of this embodiment. The number of daily non-identical doses is typically selected from 2, 3, 4, 5, 6, 7, 8, 9, 10, 11, 12, 13, 14, 15, 16, 17, 18, 19, 20, 21, 22, 23, 24, 25, 26, 27, 28, 29, and 30 consecutive non-identical daily non-identical doses.

In some embodiments, the lowest total daily dose of peanut protein is 0.1 μg, and the highest total daily dose is 5000 μg. For instance, the lowest daily dose can be between 0.1 μg and 200 μg of peanut protein, such as about 0.1 μg, about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, and about 200 μg.

Also, the highest daily dose can be between 300 and 5000 μg of peanut protein, such as about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 820 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1000 μg, about 1010 μg, about 1020 μg, about 1030 μg, about 1040 μg, about 1050 μg, about 1060 μg, about 1070 μg, about 1080 μg, about 1090 μg, about 1100 μg, about 1110 μg, about 1120 μg, about 1130 μg, about 1140 μg, about 1150 μg, about 1160 μg, about 1170 μg, about 1180 μg, about 1190 μg, about 1200 μg, about 1210 μg, about 1220 μg, about 1230 μg, about 1240 μg, about 1250 μg, about 1260 μg, about 1270 μg, about 1280 μg, about 1290 μg, about 1300 μg, about 1310 μg, about 1320 μg, about 1330 μg, about 1340 μg, about 1350 μg, about 1360 μg, about 1370 μg, about 1380 μg, about 1390 μg, about 1400 μg, about 1410 μg, about 1420 μg, about 1430 μg, about 1440 μg, about 1450 μg, about 1460 μg, about 1470 μg, about 1480 μg, about 1490 μg, about 1500 μg, about 1510 μg, about 1520 μg, about 1530 μg, about 1540 μg, about 1550 μg, about 1560 μg, about 1570 μg, about 1580 μg, about 1590 μg, about 1600 μg, about 1610 μg, about 1620 μg, about 1630 μg, about 1640 μg, about 1650 μg, about 1660 μg, about 1670 μg, about 1680 μg, about 1690 μg, about 1700 μg, about 1710 μg, about 1720 μg, about 1730 μg, about 1740 μg, about 1750 μg, about 1760 μg, about 1770 μg, about 1780 μg, about 1790 μg, about 1800 μg, about 1810 μg, about 1820 μg, about 1830 μg, about 1840 μg, about 1850 μg, about 1860 μg, about 1870 μg, about 1880 μg, about 1890 μg, about 1900 μg, about 1910 μg, about 1920 μg, about 1930 μg, about 1940 μg, about 1950 μg, about 1960 μg, about 1970 μg, about 1980 μg, about 1990 μg, about 2000 μg, about 2010 μg, about 2020 μg, about 2030 μg, about 2040 μg, about 2050 μg, about 2060 μg, about 2070 μg, about 2080 μg, about 2090 μg, about 2100 μg, about 2110 μg, about 2120 μg, about 2130 μg, about 2140 μg, about 2150 μg, about 2160 μg, about 2170 μg, about 2180 μg, about 2190 μg, about 2200 μg, about 2210 μg, about 2220 μg, about 2230 μg, about 2240 μg, about 2250 μg, about 2260 μg, about 2270 μg, about 2280 μg, about 2290 μg, about 2300 μg, about 2310 μg, about 2320 μg, about 2330 μg, about 2340 μg, about 2350 μg, about 2360 μg, about 2370 μg, about 2380 μg, about 2390 μg, about 2400 μg, about 2410 μg, about 2420 μg, about 2430 μg, about 2440 μg, about 2450 μg, about 2460 μg, about 2470 μg, about 2480 μg, about 2490 μg, about 2500 μg, about 2510 μg, about 2520 μg, about 2530 μg, about 2540 μg, about 2550 μg, about 2560 μg, about 2570 μg, about 2580 μg, about 2590 μg, about 2600 μg, about 2610 μg, about 2620 μg, about 2630 μg, about 2640 μg, about 2650 μg, about 2660 μg, about 2670 μg, about 2680 μg, about 2690 μg, about 2700 μg, about 2710 μg, about 2720 μg, about 2730 μg, about 2740 μg, about 2750 μg, about 2760 μg, about 2770 μg, about 2780 μg, about 2790 μg, about 2800 μg, about 2810 μg, about 2820 μg, about 2830 μg, about 2840 μg, about 2850 μg, about 2860 μg, about 2870 μg, about 2880 μg, about 2890 μg, about 2900 μg, about 2910 μg, about 2920 μg, about 2930 μg, about 2940 μg, about 2950 μg, about 2960 μg, about 2970 μg, about 2980 μg, about 2990 μg, about 3000 μg, about 3010 μg, about 3020 μg, about 3030 μg, about 3040 μg, about 3050 μg, about 3060 μg, about 3070 μg, about 3080 μg, about 3090 μg, about 3100 μg, about 3110 μg, about 3120 μg, about 3130 μg, about 3140 μg, about 3150 μg, about 3160 μg, about 3170 μg, about 3180 μg, about 3190 μg, about 3200 μg, about 3210 μg, about 3220 μg, about 3230 μg, about 3240 μg, about 3250 μg, about 3260 μg, about 3270 μg, about 3280 μg, about 3290 μg, about 3300 μg, about 3310 μg, about 3320 μg, about 3330 μg, about 3340 μg, about 3350 μg, about 3360 μg, about 3370 μg, about 3380 μg, about 3390 μg, about 3400 μg, about 3410 μg, about 3420 μg, about 3430 μg, about 3440 μg, about 3450 μg, about 3460 μg, about 3470 μg, about 3480 μg, about 3490 μg, about 3500 μg, about 3510 μg, about 3520 μg, about 3530 μg, about 3540 μg, about 3550 μg, about 3560 μg, about 3570 μg, about 3580 μg, about 3590 μg, about 3600 μg, about 3610 μg, about 3620 μg, about 3630 μg, about 3640 μg, about 3650 μg, about 3660 μg, about 3670 μg, about 3680 μg, about 3690 μg, about 3700 μg, about 3710 μg, about 3720 μg, about 3730 μg, about 3740 μg, about 3750 μg, about 3760 μg, about 3770 μg, about 3780 μg, about 3790 μg, about 3800 μg, about 3810 μg, about 3820 μg, about 3830 μg, about 3840 μg, about 3850 μg, about 3860 μg, about 3870 μg, about 3880 μg, about 3890 μg, about 3900 μg, about 3910 μg, about 3920 μg, about 3930 μg, about 3940 μg, about 3950 μg, about 3960 μg, about 3970 μg, about 3980 μg, about 3990 μg, about 4000 μg, about 4010 μg, about 4020 μg, about 4030 μg, about 4040 μg, about 4050 μg, about 4060 μg, about 4070 μg, about 4080 μg, about 4090 μg, about 4100 μg, about 4110 μg, about 4120 μg, about 4130 μg, about 4140 μg, about 4150 μg, about 4160 μg, about 4170 μg, about 4180 μg, about 4190 μg, about 4200 μg, about 4210 μg, about 4220 μg, about 4230 μg, about 4240 μg, about 4250 μg, about 4260 μg, about 4270 μg, about 4280 μg, about 4290 μg, about 4300 μg, about 4310 μg, about 4320 μg, about 4330 μg, about 4340 μg, about 4350 μg, about 4360 μg, about 4370 μg, about 4380 μg, about 4390 μg, about 4400 μg, about 4410 μg, about 4420 μg, about 4430 μg, about 4440 μg, about 4450 μg, about 4460 μg, about 4470 μg, about 4480 μg, about 4490 μg, about 4500 μg, about 4510 μg, about 4520 μg, about 4530 μg, about 4540 μg, about 4550 μg, about 4560 μg, about 4570 μg, about 4580 μg, about 4590 μg, about 4600 μg, about 4610 μg, about 4620 μg, about 4630 μg, about 4640 μg, about 4650 μg, about 4660 μg, about 4670 μg, about 4680 μg, about 4690 μg, about 4700 μg, about 4710 μg, about 4720 μg, about 4730 μg, about 4740 μg, about 4750 μg, about 4760 μg, about 4770 μg, about 4780 μg, about 4790 μg, about 4800 μg, about 4810 μg, about 4820 μg, about 4830 μg, about 4840 μg, about 4850 μg, about 4860 μg, about 4870 μg, about 4880 μg, about 4890 μg, about 4900 μg, about 4910 μg, about 4920 μg, about 4930 μg, about 4940 μg, about 4950 μg, about 4960 μg, about 4970 μg, about 4980 μg, about 4990 μg, and about 5000 μg.

In particular important embodiments, the treatment comprises administration of one first series of a plurality of identical daily doses which precede at least one further series of a plurality of identical daily doses, which are different than the daily doses in the first series, and which preferably are higher than the daily doses in the first series. This also constitutes an updosing scheme, but where many traditional updosing schemes have increased each new dose compared to the preceding dose, the approach described here utilises a strategy where each increase in dose is followed by repeated periodical (e.g. daily) identical doses. So in important embodiments, a plurality of series of identical daily doses are administered as an updosing phase of the allergen-specific immunotherapy, wherein the daily dose in a series of identical daily doses is higher than the daily dose in any preceding series of identical daily doses.

When such a “plurality of series-strategy” is employed, the plurality of series is selected typically from 2, 3, 4, 5, 6, 7, 8, 9, and 10 series.

Each individual series of daily administrations typically has a length (duration) in the range of 6 to 30 days, such as in the range of 6 to 22 days, for instance in the range of 6 to 16 days, and preferably about 14 days.

After completion of an updosing phase, the allergen-specific immunotherapy can be continued with a maintenance phase comprising administering a plurality of daily doses which are identical with the daily dose of the last series in the updosing phase or is in the range of ½ to 9/10 of the daily dose of the last series in the updosing phase.

As already indicated under the 2^{nd} aspect, a particular good immune response is attained with administering via the oral (and preferably sublingual) mucosa, so the allergen-specific immunotherapy preferably comprises administration to the oral mucosa, preferably by sublingual administration.

Embodiments of the 4^{th} aspect of the invention are further defined in numbered embodiments NE53 to NE69 and 127 to 129. They relate to the use of a composition according to numbered embodiments, NE1 to NE37, or to a pharmaceutical composition according to numbered embodiments NE38 to NE51 for use in a method of mitigating peanut allergy, such as treating peanut allergy in an individual in need thereof. Further embodiments, NE88 to NE94 relate to a particular dosing regimen for treating peanut allergy in a human individual in need thereof.

### Embodiments of the 4^{th} Aspect of the Invention

The 4^{th} aspect of the invention relates to a method for preparation of peanut allergen compositions having a very high degree of reproducibility due to a very (unprecedented) high level of control over the allergen content in the final product. In turn, this process enables the other aspects of the present invention.

Thus, the peanut proteins may be obtainable by a process comprising the steps of:
1) providing an aqueous extract of peanut protein obtained by extracting raw peanut kernels with an aqueous solvent to obtain an aqueous extract comprising each of of nAra h 1, nAra h 2, nAra h 3, and nAra h 6; and
2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 is eluted and collected into fractions individually enriched for nAra h 1, nAra h 2, nAra h 3, or nAra h 6; and
3) optionally collecting a flow-through fraction from the anion exchange chromatography; and
4) combining fractions or aliquots thereof as obtained in step 2 or combined step 2 and 3 to obtain said peanut proteins.

In a 4^{th} aspect, the present invention relates to a method for preparing a composition comprising two or more of nAra h 1, nAra h 2, nAra h 3, and nAra h 6, the method comprising providing 1) an extract of peanut protein obtained by extracting raw peanut kernels with an aqueous solvent to obtain an aqueous extract comprising each of nAra h 1, nAra h 2, nAra h 3, and nAra h 6; and 2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 are eluted and collected into fractions individually enriched for nAra h 1, nAra h 2, nAra h 3, or nAra h 6; and 3) optionally collecting a flow-through fraction from the anion exchange chromatography; and 4) combining two or more fractions or aliquots thereof as obtained in step 2 or in combined step 2 and 3 to obtain said peanut protein composition comprising at least two of nAra h 1, nAra h 2, nAra h 3, and nAra h 6. Preferably, fractions containing peanut protein with high molecular mass have been discarded. In this process, the peanut allergens are adsorbed to the anion exchange material and subsequently released from the material by the salt gradient elution. Fortunately, the anion exchange chromatography step can be done in preparatory scale and then suitable for industrial scale set-up.

The aqueous solvent of step 1 comprises preferably a buffered aqueous solvent having pH in the range of 7 to 9, and the salt of step 2 may be NaCl or a salt equivalent to NaCl. The conditions for carrying out step 1 is also and further disclosed for the processes described under the first aspect of the invention and are compatible with the process of the 4^{th} aspect of the invention.

It is preferred that the concentrations of nAra h 1, nAra h 2, nAra h 3, and nAra h 6 in the individual fractions obtained in step 2, such as in options i) h) and/or i) are determined, preferably by analytical RP-HPLC (cf. the examples for details on a preferred RP-HPLC quantification process). The fractions obtained in step 2 are eluted in the following order: nAra h 6, nAra h 2, nAra h 1 and nAra h 3.

The entire fractions or aliquots thereof which are combined in step 4 above, and in particularly in step 5 of option h) and/or in step 4 of option i) are in important embodiments combined to produce a molar ratio of each of the pairs nAra h 1:nAra h 2, nAra h 3:nAra h 2, nAra h 6:nAra h 2 in the range of 0.5 to 2.0, such as in the range of 0.5 to 1.5, preferably in the range of 0.6 to 1.4, such as in the range of 0.7 to 1.3. Likewise, the molar ratio of option j) of each of the pairs nAra h 1:nAra h 2, nAra h 3:nAra h 2, nAra h 6:nAra h 2 is in important embodiments in the range of 0.5 to 1.5, such as in the range of 0.6 to 1.4, such as in the range of 0.7 to 1.3.

In the method of the 4^{th} aspect, four fractions individually enriched with either nAra h 1, nAra h 2, nAra h 3, and nAra h 6 are typically collected. At least two individual fractions may be combined to provide compositions comprising two or more of the allergens selected from nAra h 1, nAra h 2, nAra h 3, and nAra h 6. For example, fractions may be combined to obtain compositions comprising nAra h 2 and nAra h 6 and with no or low amount of the two other allergens. In one embodiment, combining the four fractions or aliquots thereof provide a composition comprising each of nAra h 1, nAra h 2, nAra h 3, and nAra h 6 (i.e., a composition of the 1^{st} aspect).

The solution, which constitutes the starting point solution is normally obtained by extraction of peanut protein from raw peanut kernels, preferably defatted, optionally skinned, and pulverised kernels, with a buffered aqueous solvent having pH in the range of 6 to 9, optionally with a buffered saline aqueous solvent having pH in the range of 6 to 9, such as in the range of 6.5 to 9 or 6.5 to 8. To increase the extraction efficiency of nAra h 3, the pH might be above neutral pH, such as in the range of 7 to 9, such as in the range of 7 to 8.5, such as in the range of 7 to 8.5. In even more preferred embodiments, the pH is in the range of 7 to 8. Further details about the buffered aqueous solvent are found in the description of the 1^{st} aspect of the invention.

Fractioning of the solution by anion exchange chromatography can comprise stepwise or continuous aqueous salt gradient elution at a pH in the range of 6 to 9, whereby nAra h 1, nAra h 2, nAra h 3, and nAra h 6 are eluted and collected into the at least two individual fractions. Also, here the pH is preferably in the range of 6 to 8, but preferably above pH 7 to ensure that the proteins possess a negative charge to be efficiently adsorbed to the anion exchange material. Optimal conditions are obtained with a pH in range of 7 to 8, such as in the range of 7.2 to 7.8.

The concentrations of the four allergens nAra h 1, nAra h 2, nAra h 3, and nAra h 6 in the at least two fractions are preferably quantified by RP-HPLC to determine the aliquots required to combine the at least to fractions for providing a composition containing controlled amounts or pre-selected amounts of two or more of the four allergens. The RP-HPLC method is further specified as described for the 1^{st} aspect of the invention.

In some embodiments, the process comprises the steps of 1) extracting peanut protein from raw peanut kernels with an aqueous solvent; 2) subjecting the extracted protein to anion exchange chromatography with stepwise or continuous aqueous salt gradient elution at a pH in the range of 7 to 9, whereby each of nAra h 1, nAra h 2, nAra h 3, and nAra h 6 is eluted and collected into individual fractions; 3) optionally collecting a flow-through fraction from the anion exchange chromatography; and 4) combining the entire fractions or aliquots of two or more of the fractions obtained in step 2 and optionally step 3, to obtain a composition comprising two or more of nAra h 1, nAra h 2, nAra h 3, and nAra h 6. The concentrations of the four allergens nAra h 1, nAra h 2, nAra h 3, and nAra h 6 in the fractions of step 2 are conveniently quantified by RP-HPLC to determine the aliquots of relevant fractions that should be combined to provide a composition comprising the two or more peanut allergens in pre-selected/controlled amounts.

In some embodiments where it is desirable to obtain compositions comprising each of nAra h 1, nAra h 2, nAra h 3, and nAra h 6, the process comprises the steps of: 1) extracting peanut protein from raw peanut kernels with an aqueous solvent; 2) subjecting the extracted protein to anion exchange chromatography with stepwise or continuous aqueous salt gradient elution at a pH in the range of 7 to 9, whereby each of nAra h 1, nAra h 2, nAra h 3, and nAra h 6 is eluted and collected into individual fractions; 3) optionally collecting a flow-through fraction from the anion exchange chromatography; and 4) combining the entire fractions or aliquots of each fraction obtained in step 2 and optionally step 3, to obtain a composition comprising each of nAra h 1, nAra h 2, nAra h 3, and nAra h 6. The concentrations of the four allergens nAra h 1, nAra h 2, nAra h 3, and nAra h 6 in the fractions of step 2 are conveniently quantified by RP-HPLC to determine the aliquots of each fraction that should be combined to provide a composition containing controlled amounts or pre-selected amounts of each of the four allergens.

In some embodiments, the fractions or aliquots thereof are combined to obtain a composition comprising each of nAra h 1, nAra h 2, nAra 3 and nAra h 6 in the composition, provided that the molar ratio of each of the pairs nAra h 1:nAra h 2, nAra h 3:nAra h 2, nAra h 6:nAra h 2 is in the range of 0.5 to 1.5 when determined by analytical scale RP-HPLC, preferably the molar ratio of each of the pairs are in the range of 0.6 to 1.4, such as in the range of 0.7 to 1.3.

In a particular important embodiment of the method of the 4^{th} aspect, fractions containing peanut protein with high molecular mass have been discarded by discarding fractions eluting later than the enriched fractions of nAra h 1, nAra h 2, nAra h 3, and nAra h 6. This hence attains the goal of avoiding the high molecular mass aggregates discussed supra in association with the 1^{st} aspect of the invention. Thus, high molecular mass molecules with a mass above 700 kDa have been discarded.

The aqueous solvent mentioned above is further as specified for the aqueous solvent described under the 1^{st} aspect of the invention. The aqueous solvent typically comprises TRIS in a molar range of 10 to 200 mM, preferably in the range of 10 to 100 mM, such as in the range of 10 to 50 mM, and optionally comprises NaCl or an equivalent salt in an amount in the range of 5 to 200 mM, preferably in the range of 10 to 100 mM, 10 to 50 mM.

The stepwise or continuous aqueous salt gradient elution is preferably carried out using NaCl as the salt or a salt equivalent to NaCl. Equivalent salts are further specified under the 1^{st} aspect of the invention. As shown in the examples, this entails the advantage that the four relevant allergens are eluted at low salt concentrations and the high mass aggregates elute at a higher salt concentration.

Further details about the preparation method (e.g., about anion exchange material, peanut source, extraction solvents, solvents for elution of the allergens adsorbed to the anion exchange material etc.) are found in the description of the 1^{st} aspect of the invention.

Further embodiments are described in numbered embodiments NE70 to NE81, which relates to a method of preparing a composition comprising two or more of nAra h 1, nAra h 2, nAra h 3, and nAra h 6, and which are suitable for producing compositions comprising a pre-selected quantity of one or more of nAra h 1, nAra h 2, nAra h 3, and nAra h 6.

### Embodiments of the 5^{th} Aspect of the Invention

A 5^{th} aspect of the present invention relates to a kit comprising a sealed package comprising a plurality of separate compartments, each compartment comprising a unit dose form of the pharmaceutically acceptable formulation of the 2^{nd} aspect of the invention (or any embodiments thereof disclosed herein), wherein at least one unit dose form comprises a quantity of total peanut protein.

In some embodiments of the kit of the 5^{th} aspect of the invention, at least one dose is unique, and preferably no unit doses are identical. Such a kit is particularly suitable for traditional updosing scheme, where all doses are increasing.

In other embodiments, a first plurality of unit doses is identical, and wherein at least one further plurality of unit doses is identical but higher than the unit doses in the first plurality of unit doses. It will be understood that this kit is useful in the above-disclosed “plurality-of-series”-updosing regimens. In preferred embodiments of this type of kit, at least 3 pluralities of identical unit doses are included, each comprising unit doses that are different from the unit dose in any of the other pluralities of unit doses. The at least 3 pluralities can for example be selected from 3, 4, 5, 6, 7, 8, 9, 10, 11, 12, 13, 14, and 15.

The quantity of peanut protein in each unit dose form is preferably as defined for the daily doses disclosed above in the context of the 3^{rd} aspect of the invention. Further embodiments are described under numbered embodiments NE82-87, which relates to a kit comprising a sealed package comprising the unit doses suitable for being used in the methods of mitigating peanut allergy. Each unit dose may comprise a composition according to NE1 to NE37 or a pharmaceutical composition according to NE38 to NE51.

### Embodiments of the 6^{th} Aspect of the Invention

A sixth aspect of the invention relates to a composition of the first aspect or 2^{nd} aspect of the invention for use in a therapeutically effective amount in a method of treating a human individual against peanut allergy. In other words, the sixth aspect of the invention relates to the use of a composition of the first aspect or 2^{nd} aspect of the invention for the preparation of a medicament for treating a human against peanut allergy.

The 6^{th} takes advantage of the 1^{st}, 2^{nd}, 4^{th} and 5^{th} aspects of the invention and generally constitutes a method for treating peanut allergy by the use of the conditions discussed in the 3^{rd} aspect.

The term “treatment of peanut allergy” is meant to include the mitigation and/or elimination, amelioration, inhibition, slowing down the progression or severity of one or more symptoms or clinical signs of peanut allergy. By example, the term “treatment of allergy” may designate the mitigation of peanut allergy, including peanut-allergen induced anaphylaxis (e.g., said anaphylaxis being caused by exposure to peanuts or peanut containing products). The mitigation of peanut allergy, including peanut allergen-induced anaphylaxis may in particular be to tolerate accidental exposure to peanut. The term “treatment” can also include prophylactic treatment, which includes a delay in the onset or the prevention of the onset of one or more symptoms or clinical signs of peanut allergy in a patient that previously have experienced symptoms or clinical signs of peanut allergy. Treatment is generally considered “effective” if one or more symptoms or clinical signs are reduced or improved. Alternatively, treatment is “effective” if the progression of the allergy is reduced or halted, such as cessation of, or at least slowed down in the progression or worsening of symptoms or clinical signs compared to what would be expected in the absence of treatment. Beneficial or desired efficacy of the treatment may also include diminishment or reduced frequency of serious immune reactions like anaphylaxis including anaphylactic shock. For example, a beneficial effect may be the reduced need for epinephrine administration to mitigate anaphylaxis.

Accidental exposure to peanut is typically occurring without the individuals own desire to eat peanut or to be exposed to peanut, which then might trigger an allergic response. The individual may be exposed to whole peanuts or products containing peanuts or peanut proteins.

Clinical sign of peanut allergy may include vomiting, stomach cramps, indigestion, diarrhea, wheezing, shortness of breath, difficulty breathing, repetitive cough, tightness in throat, hoarse voice, weak pulse, pale or blue coloring of the skin, hives, swelling which can affect the tongue and/or lips, dizziness, and confusion and in severe cases anaphylaxis, anaphylactic shock. Anaphylaxis is a life-threatening whole-body (systemic) response to an allergen and symptoms may include impaired breathing, swelling in the throat, a sudden drop in blood pressure, pale skin or blue lips, fainting and dizziness. Anaphylaxis should be treated immediately with epinephrine.

Thus, in embodiments of the sixth aspect, the peanut protein compositions disclosed herein are used in a therapeutically effective amount for mitigation of peanut allergy and/or peanut allergen-induced anaphylaxis caused by accidental exposure to one or more peanut allergens; one or more peanuts; peanut protein; or a peanut protein-containing product in a human individual in need thereof. In embodiments thereof the peanut protein compositions are used for conducting allergen-specific immunotherapy, which requires repeated administration of the specific allergen causing the allergy over a period and optionally initiated by an updosing phase to reach a high tolerable dose for continued treatment (maintenance phase).

The human individual may be any human individual in need of treatment, such a child, adolescence or an adult. Typically, the human individual is proven sensitized or allergic to peanut by conducting oral food challenge and/or skin-prick testing with peanut allergen extracts. In addition, the individual may present with detectable specific IgE against peanut allergens of a peanut allergen extract or against one or more of the four peanut allergens. The allergen specific IgE antibodies may be detected in blood samples and with a concentration about 0.7 kU/ml.

The efficacy of the treatment may be realised by following the unique biomarkers for allergy. For example, efficacious treatment may be realised by reduced peanut allergen specific IgE levels in the blood and/or increase in peanut-allergen specific IgG4 levels in the blood following treatment. That is to say that treatment of peanut allergy comprises increasing the concentration of peanut-allergen specific IgG4 levels in the blood or other biological secretes (saliva, nasal or lung lavage) compared to before treatment and/or increasing the ratio between peanut-allergen specific IgE and IgG4 levels in the blood or other biological secretes compared to before treatment. Typically, the increase can be observed at the end of the updosing phase, or after at least three months treatment. The peanut allergen specific levels may be determined in respect of one or more of the four key peanut allergens (Ara h 1, 2, 3 and 6) or in terms of whole peanut extract.

The treatment of peanut allergy may comprise induction of tolerance, such as immunological tolerance, to the ingestion of one or more peanut allergens; one or more peanuts; peanut protein; or a peanut protein-containing product. The aim of the tolerance induction is to tolerate accidental exposure to a peanut protein containing product. The tolerance induction might be realised when the individual can eat at least 300 mg peanut protein without having a severe allergic reaction. The test for tolerance of peanut protein can be assessed by an oral food challenge test.

Typically, the treatment requires daily, weekly, biweekly, or monthly administration dependent on the route of administration. For sublingual administration, the treatment comprises daily administration, preferably daily administration of a single dose. The concentration of doses is typically expressed as the concentration of peanut protein and with the lowest daily dose being 0.1 μg of peanut protein, and the highest daily dose being 5000 μg of peanut protein.

A typical dosing regimen comprises the administration of one first series of a plurality of identical daily doses which precedes at least one further series of a plurality of identical daily doses, which are different than the daily doses in the first series, and which preferably are higher than the daily doses in the first series. Optionally, the plurality of series of identical daily doses are administered as an updosing phase, wherein the daily dose in a series of identical daily doses is higher than the daily dose in any preceding series of identical daily doses.

In some embodiments, the lowest daily dose of peanut protein is in the range of 1-150 μg. The updosing phase may comprise steps with administering three to ten doses, preferably in increasing doses. Therefore, the plurality of series may be selected from 3, 4, 5, 6, 7, 8, 9, and 10 series. Typically, the same dose is administered for 6-22 days before administering a larger dose. Therefore, the series has a duration in the range of 6 to 22 days.

After completion of the updosing phase, the treatment is continued with a maintenance phase comprising administering a plurality of daily doses which are identical to the daily dose of the last series in the updosing phase or is in the range of ½ to 9/10 of the daily dose of the last series in the updosing phase. The daily dose of peanut protein administered in the maintenance phase may in the range of 300 to 5000 μg.

Typically, the dosage is for administration to the oral mucosa, such as to the sublingual mucosa—the latter being preferred.

In some embodiments, the method comprises multiple daily administrations of non-identical doses of peanut allergen, optionally preceding multiple daily administrations of identical daily doses of peanut allergen.

In other, to some degree overlapping, embodiments, the method comprises multiple daily administrations of identical doses of peanut allergen, optionally preceded by multiple daily administrations of non-identical doses of peanut allergen.

It is to be noted that daily administrations are not a prerequisite according to the invention—other than 1-day intervals between each dose can be employed, but daily doses are preferred, and in particular single daily (once-daily) administrations are preferred.

In yet other and important embodiments, the method comprises administrations of a plurality of series of identical daily doses of peanut allergen or peanut protein, wherein the daily doses in at least one series are non-identical with the daily doses in another series. Preferably, each of the plurality of series comprises daily doses that are different from the doses in any other of the series in the plurality of series, and wherein each series following an earlier series comprises higher doses than the earlier series. The plurality of series is typically constituted by 2, 3, 4, 5, 6, 7, 8, 9, 10, 11, 12, 13, 14, or 15 series.

The 6^{th} aspect also includes a method of mitigation of peanut allergy and/or peanut-induced anaphylaxis in a human by allergen-specific immunotherapy, the method comprising an updosing phase and optionally a maintenance phase, wherein the updosing phase comprises multiple consecutive series of administering a daily dose of peanut protein composition to the oral mucosa, wherein the daily dose within each series is identical and wherein any dose in a preceding series is lower than in a subsequent series and wherein each series has a duration length ranging from 6 to 30 days; and wherein
the daily dose administered in the first series contains a total amount of peanut protein in the range of 0.1 μg to 200 μg;
the daily dose of the last series contains a total amount of peanut proteins in the range of 300 μg to 5000 μg; and
wherein the number of series is in the range from 2 to 9, such as in the range of 3 to 7, such as particularly, 3, 4, 5, 6, 7, 8, or 9, preferably 3, 4, or 5.

In other words, the 6^{th} aspect also relates to a pharmaceutical composition for use in a method of mitigation of peanut allergy and/or peanut allergen-induced anaphylaxis in a human by allergen-specific immunotherapy or the use of a pharmaceutical compositions for the manufacturing of a medicament for use in a method of mitigation of peanut allergy and/or peanut allergen-induced anaphylaxis in a human by allergen-specific immunotherapy.

Thus, this part of the 6^{th} aspect of the invention focusses on the clinically important updosing phase which initiates the allergen-specific immunotherapy, including the peanut allergens as well on the concentrations and conformations of the allergens administered. The updosing phase might be followed by a maintenance phase, which preferably comprises administration with the same pharmaceutical composition and with the same route of administration as used within the updosing phase. However, the maintenance phase might in the alternative be conducted by oral allergen immunotherapy (OIT), subcutaneous allergen immunotherapy, or even by intake of peanuts or peanut-containing products.

The peanut protein composition is formulated into a pharmaceutical composition by use of a pharmaceutically acceptable carrier, diluent, excipient, or vehicle and the peanut protein comprises allergens extracted or extractable from raw peanut kernels (preferably, pulverised defatted peanut kernels) by an aqueous solvent, wherein the allergens at least comprise each of nAra h 1, nAra h 2, nAra h 3, and nAra h 6.

With the purpose to increase the peanut-allergen specific IgG4 blood levels for all four key allergens to the same extent during treatment, the peanut protein composition preferably comprise a molar ratio of each of the pairs nAra h 1:nAra h 2, nAra h 3:nAra h 2, and nAra h 6:nAra h 2 in the range 0.5-2.0, such as in the range of 0.5-1.5 or in a more narrow range disclosed herein. Thus, in embodiments of the 6^{th} aspect, the molar concentration ratios of each of the pairs nAra h 1:nAra h 2, nAra h 3:nAra h 2, and nAra h 6:nAra h 2 is in the range of 0.5-2.0, such a 0.5-1.5 or more narrow. The molar concentration for each of nAra h 1, nAra h 2, nAra h 3, and nAra h 6 are meant to be expressed as the concentration of the monomer polypeptide. By the “monomer polypeptide” is meant the polypeptide (characterized by one or more amino acid sequences derived from isoforms of the allergen) of which each of the allergen proteins is constituted. So, where the natural allergen can appear as a di-, tri-, or other multimer of polypeptides, it will always be constituted by single polypeptides. As explained under the 1^{st} aspect of the invention, such molar concentrations may be determined by analytical scale reverse phase HPLC.

The concentration of nAra h 2 on the peanut composition may range from 50-150 μg per mg peanut protein and the amount of nAra h 3 may range from 160-500 μg per mg peanut protein. The four allergens nAra h 1, nAra h 2, nAra h 3, and nAra h 6 in combination may constitute at least 75% by weight of the peanut protein to be able to provide high doses of peanut proteins with therapeutically relevant allergens. Preferably, the four allergens may constitute at least 80%, such as at least 85%, such as at least 90% by weight of the peanut protein. Where other peanut allergens are desirable to be administered, the four allergens may constitute at the most 98% of the weight of the peanut protein.

At any rate, in important embodiments herein, the peanut protein administered is essentially free from peanut protein having a molecular mass of at least 700 kDa as disclosed for peanut compositions of the of the 1^{st} aspect.

Preferably the peanut protein composition mentioned above is a composition of the 1^{st} aspect of the invention and any embodiments thereof disclosed herein, or a pharmaceutically acceptable formulation of the 2^{nd} aspect of the invention and any embodiments thereof disclosed herein.

If series of identical doses are administered as part of the method of the 6^{th} aspect, the series each preferably has a duration of 10-21 days, preferably about 14 days. In this context, the lowest daily doses in the first series discussed under the 3^{rd} aspect of the invention apply mutatis mutandis to daily doses in the first series in the 6^{th} aspect, and likewise the daily doses in the last series can be those discussed above as the highest daily doses in the 3^{rd} aspect of the invention. The pharmaceutical composition for the use according to any one of claims 86 to 91, wherein each of the series has a duration of 10-21 days.

If series of identical doses are administered, the daily dose of a series later than the first series is preferably increased by a factor of 2 to 4 compared to the daily dose of the directly preceding series, such as a factor between 3 and 3.5, such a as between 2 and 3.

In an embodiment, the daily dose of the first series is about 1 μg and the daily dose of the last series is about 4320 μg, and the number of series is 9, the doses of the 7 series between the first and last series are in escalating order about 3 μg, about 10 μg, about 40 μg, about 120 μg, about 360 μg, about 1080 μg, and about 2160 μg, respectively.

In an embodiment, the daily dose of the first series is about 3 μg and the daily dose of the last series is about 4320 μg, and the number of series is 8, the doses of the 6 series between the first and last series are in escalating order about 10 μg, about 40 μg, about 120 μg, about 360 μg, about 1080 μg and about 2160 μg, respectively.

In an embodiment, the daily dose of the first series is about 10 μg and the daily dose of the last series is about 4320 μg, and the number of series is 7, the doses of the 5 series between the first and last series are in escalating order about 40 μg, about 120 μg, about 360 μg, about 1080 μg and about 2160 μg, respectively.

In an embodiment, the daily dose of the first series is about 40 μg and the daily dose of the last series is about 4320 μg, and the number of series is 6, the doses of the 4 series between the first and last series are in escalating order about 120 μg, about 360 μg, about 1080 μg and about 2160 μg, respectively.

In an embodiment, the daily dose of the first series is about 120 μg and the daily dose of the last series is about 4320 μg, and the number of series is 5, the doses of the 3 series between the first and last series are in escalating order about 360 μg, about 1080 μg and about 2160 μg, respectively.

In an embodiment, the daily dose of the first series is about 1 μg and the daily dose of the last series is about 2160 μg, and the number of series is 8, the doses of the 6 series between the first and last series are in escalating order about 3 μg, about 10 μg, about 40 μg, about 120 μg, about 360 μg and about 1080 μg, respectively.

In an embodiment, the daily dose of the first series is about 3 μg and the daily dose of the last series is about 2160 μg, and the number of series is 7, the doses of the 5 series between the first and last series are in escalating order about 10 μg, about 40 μg, about 120 μg, about 360 μg and about 1080 μg, respectively.

In an embodiment, the daily dose of the first series is about 10 μg and the daily dose of the last series is about 2160 μg, and the number of series is 6, the doses of the 4 series between the first and last series are in escalating order about 40 μg, about 120 μg, about 360 μg and about 1080 μg, respectively.

In an embodiment, the daily dose of the first series is about 40 μg and the daily dose of the last series is about 2160 μg, and the number of series is 5, the doses of the 3 series between the first and last series are in escalating order about 120 μg, about 360 μg and about 1080 μg, respectively.

In an embodiment, the daily dose of the first series is about 120 μg and the daily dose of the last series is about 2160 μg, and the number of series is 4, the doses of the 2 series between the first and last series are in escalating order about 360 μg and about 1080 μg, respectively.

In an embodiment, the daily dose of the first series is about 1 μg and the daily dose of the last series is about 1080 μg, and the number of series is 7, the doses of the 5 series between the first and last series are in escalating order about 3 μg, about 10 μg, about 40 μg, about 120 μg and about 360 μg, respectively.

In an embodiment, the daily dose of the first series is about 3 μg and the daily dose of the last series is about 1080 μg, and the number of series is 6, the doses of the 4 series between the first and last series are in escalating order about 10 μg, about 40 μg, about 120 μg and about 360 μg, respectively.

In an embodiment, the daily dose of the first series is about 10 μg and the daily dose of the last series is about 1080 μg, and the number of series is 5, the doses of the 3 series between the first and last series are in escalating order about 40 μg, about 120 μg and about 360 μg, respectively.

In an embodiment, the daily dose of the first series is about 40 μg and the daily dose of the last series is about 1080 μg, and the number of series is 4, the doses of the 2 series between the first and last series are in escalating order about 120 μg and about 360 μg, respectively.

In an embodiment, the daily dose of the first series is about 120 μg and the daily dose of the last series is about 1080 μg, and the number of series is 3, the doses of the 1 series between the first and last series are about 360 μg.

The daily dose in the embodiments above is meant to denote the concentration of peanut protein in the administered composition. The concentration of the peanut protein in the composition may be determined by amino acid analysis (AAA) or by the Bradford protein assay using Bovine Serum Albumin as reference standard (BCA), preferably by amino acid analysis (AAA).

As mentioned above, allergen-specific immunotherapy has proven particularly effective, when the allergen composition is administered to the oral mucosa, in particular the sublingual mucosa. Hence, preferred administration to the oral mucosa is by buccal or sublingual administration, preferably sublingual administration.

The method of the 6^{th} aspect can further comprise a maintenance phase, which comprises a plurality of administrations of peanut protein doses to the oral mucosa, preferably the sublingual mucosa, with at least one day apart. Preferably, the dose of total peanut protein in the maintenance phase is identical to the daily dose of any last series of administrations or is in the range of ½ to 9/10 of the daily dose of any last series.

To gauge the efficiency of the method, the patient's allergic reaction is measured in response to challenge with the offending allergen (in this case peanuts or products containing material derived from peanuts, e.g. peanut flour). It is preferred that the human individual after completion of the updosing phase is able to tolerise at least 300 mg peanut protein in an oral food challenge test, such as at least 400 mg, 500 mg, 600 mg, 700 mg, 800 mg, 1000 mg, 1200 mg, 1500 mg, 2000 mg, 2500 mg, 3000 mg, 4000 mg, 5000 mg, or 6000 mg peanut protein. Likewise, it is preferred that the human individual after completion of the updosing phase and at least six months of maintenance phase is able to tolerise at least 300 mg peanut protein in an oral food challenge test, such as at least 400 mg, 500 mg, 600 mg, 700 mg, 800 mg, 1000 mg, 1200 mg, 1500 mg, 2000 mg, 2500 mg, 3000 mg, 4000 mg, 5000 mg, or 6000 mg peanut protein.

The maintenance phase may be upheld until the human acquires sustained responsiveness meaning that the human individual may be able to tolerise at least 300 mg peanut protein in an oral food challenge test after end of maintenance phase or updosing phase.

The maintenance phase may be upheld until the human acquires sustained responsiveness meaning that the human individual may be able to tolerize at least 300 mg peanut protein in an oral food challenge test after end of maintenance phase or updosing phase. Sustained responsiveness might be realized when the human individual can tolerize ingestion of at least 300 mg peanut protein, such as at least 600 mg 700 mg, 800 mg, 1000 mg, 1200 mg, 1500 mg, 2000 mg, 2500 mg, 3000 mg, 4000 mg, 5000 mg, or 6000 mg peanut protein after treatment cessation for three months or more, such as 6 months, 1, 2 or 4 years. The maximum dose tolerated may be determined as described by Davis et al. (2022).

Further or similar embodiments of the 6^{th} aspect can be found in numbered embodiments NE95 to NE125, which relate to a method of mitigating peanut allergy in an individual thereof, wherein a peanut composition comprising each of the four allergens nAra h 1, nAra h 2, nAra h 3, and nAra h 6 are administered in certain dosing regimens, which comprises an updosing phase with escalating doses administered within a shorter period. Such dosing regimen are considered safe without incurring serious adverse events, which could require epinephrine injection.

### NUMBERED EMBODIMENTS OF THE PRESENT INVENTION

In particular, the present invention relates to the subject matter defined in the following numbered embodiments. It will be understood that these numbered embodiments serve the same purpose as patent claims for the purpose of defining the subject matter of the invention, including the possible combination of features derivable by the combination of several claims, but that the subject matter in the numbered embodiments shall not be interpreted as being part of the claimed subject matter, unless such subject matter is or becomes recited in the claims.

NE1. A composition comprising each of nAra h 1, nAra h 2, nAra h 3, and nAra h 6, wherein said composition is characterized by one or more features a) to k), wherein the features are:
a. being essentially free from peanut protein having a molecular mass of ≥700 kDa. The molecular mass may be determined by analytical scale size exclusion HPLC; and/or
b. an aqueous sample of the composition can be loaded onto a reverse phase HPLC column and eluted to separate nAra h 1, nAra h 2, nAra h 3, and nAra h 6 into quantifiable polypeptides when the reverse phase HPLC column is subjected to elution by mixed isocratric and gradient elution, which comprises mixing an eluent A consisting of aqueous 0.1% trifluoroacetic acid with increasing volumes of an eluent B consisting of acetonitrile mixed with 0.1% trifluoroacetic acid; and/or
c. being essentially free from peanut protein, which due to molecular size constraints is incapable of being loaded and/or separated in a reverse phase HPLC column. That is to say that nAra h1, nAra h 2, n Ara h 3, and nAra h 6 preferably are in non-denatured conformations, such as non-aggregated conformations; and/or
d. being essentially free from high molecular weight complexes of peanut-derived protein, where said high molecular complexes are characterised by being present in a discarded fraction, which can be obtained by extraction of peanut protein from raw peanut kernels (preferably pulverized raw peanut kernels) with an aqueous solvent to obtain an aqueous extract comprising each of of nAra h 1, nAra h 2, nAra h 3, and nAra h 6, subsequently subjecting the aqueous extract of extracted peanut proteins to preparative scale anion exchange chromatography with stepwise or continuous aqueous salt gradient elution at a pH in the range of 7 to 9, whereby each of nAra h 1, nAra h 2, nAra h 3, and nAra h 6 are eluted, wherein the gradient elution is continued with higher salt concentrations after elution of each of nAra h 1, nAra h 2, nAra h 3, and nAra h 6, whereby said discarded fraction is a fraction eluted after nAra h 1, nAra h 2, nAra h 3, and nAra h 6 or is retained by the anion exchange chromatography; and/or
e. comprising a controlled concentration of nAra h 3 and/or nAra h 2; and/or
f. comprising a controlled concentration of each of nAra h 1, nAra h 2, nAra h 3, nAra h 6; and/or
g. being obtained or obtainable by a process comprising the steps of i) extracting peanut protein from raw peanut kernels, preferably from pulverized raw peanut kernels, with an aqueous solvent to obtain an aqueous extract comprising each of of nAra h 1, nAra h 2, nAra h 3, and nAra h 6 and ii) purifying the extracted proteins by anion exchange chromatography, said chromatography comprises loading said aqueous extract to an anion exchange material and eluting with a salt gradient elution to collect fractions individually enriched for nAra h 1, nAra h 2, nAra h 3, or nAra h 6 and iii) combine fractions or aliquots thereof individually enriched with one or more of nAra h 1, nAra h 2, nAra h 3, and nAra h 6 and preferably to discard fractions eluting later than the enriched fractions of nAra h 1, nAra h 2, nAra h 3, and nAra h 6; and/or
h. being obtained or obtainable by a process comprising the steps of:
   1) extracting peanut protein from raw peanut kernels, preferably from pulverized raw peanut kernels, with an aqueous solvent to obtain an aqueous extract comprising each of of nAra h 1, nAra h 2, nAra h 3, and nAra h 6; and
   2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 are eluted and collected into one or more individual fractions; and
   3) optionally collecting a flow-through fraction from the anion exchange chromatography; and
   4) optionally continuing the stepwise or continuous salt gradient after the elution of each of nAra h 1, nAra h 2, nAra h 3, and nAra h 6 to obtain a fraction to be discarded; and
   5) combining fractions or aliquots thereof obtained in step 2, optionally obtained in both step 2 and step 3, to obtain said composition; and/or
i. being obtained or obtainable by a process comprising the steps of:
   1) extracting peanut protein from raw peanut kernels, preferably from pulverized raw peanut kernels) with an aqueous solvent to obtain an aqueous extract comprising each of of nAra h 1, nAra h 2, nAra h 3, and nAra h 6; and
   2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 are eluted and collected into fractions individually enriched for nAra h 1, nAra h 2, nAra h 3, or nAra h 6;
   3) optionally collecting a flow-through fraction from the anion exchange chromatography; and
   4) combining fractions or aliquots thereof obtained in step 2, optionally obtained in both step 2 and step 3, to obtain said composition; and/or
j. comprising molar ratios nAra h 1:nAra h 2, nAra h 3:nAra h 2, nAra h 6:nAra h 2, which each are in the range between 0.5 to 2.0, such as between 0.5 and 1.5. each of the peanut proteins nAra h 1, nAra h 2, nAra h 3, and nAra h 6 may be extractable from raw peanuts by an aqueous solvent. The concentration of each of the allergens may be quantified by analytical scale RP-HPLC and/or LC-MS/MS. For converting mass units to molar concentrations, the following molar masses may be used: 68757 g/mol for Ara h 1, the molar mass of 17994 g/mol for Ara h 2, the molar mass of 58600 g/mol for Ara h 3 and the molar mass of 14846 g/mol for Ara h 6; and/or
k. being enriched for total amount of water-soluble peanut proteins selected from nAra h 1, nAra h 2, nAra h 3, and nAra h 6 per weight unit total peanut protein compared to an aqueous extract of pulverized raw peanut kernels that have been subjected to extraction with an aqueous solvent.

NE2. The composition according to numbered embodiment NE1, wherein the analytical scale size exclusion HPLC of option a) is capable of separating the size-indicating reference standards thyroglobulin (670 kDa), bovine γ-globulin (158 kDa), chicken ovalbumin (44 kDa), equine myoglobin (17 kDa), and vitamin B12 (1.35 kDa) by elution with aqueous (phosphate) buffered saline having pH in the range of 7 to 7.5.

NE3. The composition according to numbered embodiment NE1 or NE2, wherein the composition of option a) which is being essentially free from peanut protein having a molecular mass >900, such as >800, such as >700 kDa, wherein the peanut proteins with molecular mass >700 kDa is determined by subjecting an aqueous sample of the composition to analytical scale size exclusion HPLC method, which method is capable of separating the size-indicating reference standards thyroglobulin (670 kDa), bovine γ-globulin (158 kDa), chicken ovalbumin (44 kDa), equine myoglobin (17 kDa), and vitamin B12 (1.35 kDa) by elution with aqueous buffered(preferably phosphate buffered) saline having pH in the range of 7 to 7.5 and determining that the resulting chromatogram is essentially free from peanut protein peaks eluting with a mass similar to or higher than the size-indicating reference standard thyroglobulin having a mass of 670 kDa AND/OR wherein the presence of peanut proteins with molecular mass >700 kDa is determined by subjecting an aqueous sample of the composition to native gel electrophoresis.

NE4. The composition according to any one of the preceding numbered embodiments, wherein the composition is being essentially free from peanut protein having a molecular mass >650 kDa, preferably >600 kDa, such as >550 kDa, >500 kDa, >450 kDa, >450 kDa, >400 kDa, said molecular mass being determined by analytical scale size exclusion HPLC.

NE5. The composition according to any one of the preceding numbered embodiments, wherein the extraction of peanut protein from raw peanut kernels with an aqueous solvent in option d), g), step 1 of option h) or step 1 of option i) comprises extraction with a buffered aqueous solvent having pH in the range of 6 to 9, optionally with a buffered saline aqueous solvent having pH in the range of 6 to 9.

NE6. The composition according to numbered embodiment NE5, wherein the pH of the buffered aqueous solvent is in the range of 6.5 to 8.5, such as in the range of 6.5 to 9, such as in the range of 6.5 to 8, such as in the range of 7 to 9, such as in the range of 7 to 8.5, such as in the range of 7 to 8.5, and preferably in the range of 7 to 8.

NE7. The composition according to numbered embodiment NE5 or NE6, wherein the aqueous solvent comprises TRIS in a molar range of 10 to 200 mM, preferably in the range of 10 to 100 mM, such as in the range of 10 to 50 mM, and optionally comprises NaCl or an equivalent salt in an amount in the range of 5 to 200 mM, preferably in the range of 10 to 100 mM, such as in the range of 10 to 50 mM.

NE8. The composition according to any one of numbered embodiments NE5-NE7, wherein the aqueous solvent is 50 mM TRIS+50 mM NaCl dissolved in purified water and pH adjusted to 7.4 with 2.0 M NaOH.

NE9. The composition according to any one of the preceding numbered embodiments, wherein the stepwise or continuous aqueous salt gradient elution in option d), g), step 2 of option h) or step 2 of option i) is carried out at a pH in the range of 7 to 8.5, preferably in the range of 7 to 8, such as in the range of 7.2 to 7.8.

NE10. The composition according to any one of the preceding numbered embodiments, wherein the stepwise or continuous aqueous salt gradient elution in option d), g), step 2 of option h) or step 2 of option i) is carried out using NaCl as the salt or a salt equivalent to NaCl.

NE11. The composition according to any one of the preceding numbered embodiments, wherein the controlled concentration of nAra h and/or nAra h 2 in option(s) e) and/or f) is determined by means of a quantitative immune assay, analytical scale reverse phase HPLC or quantitative LC-MS/MS, preferably by analytical scale reverse phase HPLC.

NE12. The composition according to any one of the preceding numbered embodiments, wherein the controlled concentration of nAra h 3 and/or nAra h 2 in option(s) e), f) and/or j) is determined by analytical scale reverse phase HPLC comprising separation of nAra h 1, nAra h 2, nAra h 3, and nAra h 6 by use of mixed isocratic and gradient elution, said gradient elution comprises mixing an eluent A consisting of aqueous 0.1% trifluoroacetic acid with increasing volumes of an eluent B consisting of acetonitrile with 0.1% trifluoroacetic acid and quantification against pure calibration standard of nAra h 3, optionally converting the concentration by weight of nAra h 3 in the composition to molar concentration of nAra h 3 by using a molar mass of 58600 g/mol for Ara h 3.

NE13. The composition according to any one of the preceding numbered embodiments, wherein the controlled concentration of nAra h 3 in option(s) e) and/or f) is in the range of 12% to 70% by weight of the total mass of peanut proteins in the composition, such as in the range of 12% to 60%, such as in the range of 15% to 60%, such as in the range of 20% to 60%, such as in the range of 25 to 55%, such as in the range of 15% to 50%, such as in the range of 25% to 50%, such as in the range of 17% to 53%.

NE14. The composition according to any one of the preceding numbered embodiments, wherein the controlled concentration of nAra h 3 in option(s) e) and/or f) is in the range of 18% to 46% by weight of the total mass of peanut proteins in the composition.

NE15. The composition according to any one of the preceding numbered embodiments, wherein the controlled concentration of nAra h 3 in option(s) e) and/or f) is in the range of 21% to 42% by weight of the total mass of peanut proteins in the composition.

NE16. The composition according to any one of the preceding numbered embodiments, wherein the controlled concentration of nAra h 3 and/or nAra h 2 in option(s) e) and/or f) is in the range of 2-12 nmol/mg of the total mass of peanut proteins, such as in the range of 3-11 nmol/mg, such as in the range of 4-10 nmol/mg, such as in the range of 4-9 nmol/mg, such as in the range of 4-8 nmol/mg, such as in the range of 2.8 nmol/mg to 8.4 nmol/mg of the total mass of peanut proteins in the composition, preferably in the range of 3.1 nmol/mg to 7.8 nmol/mg, such as in the range of 3.6 nmol/mg to 7.1 nmol/mg of the total mass of peanut proteins in the composition.

NE17. The composition according to any one of the preceding numbered embodiments, wherein the concentration of nAra h 1, nAra h 2, nAra h 3 and nAra h 6 in option f) is determined by means of a quantitative immune assay, analytical scale reverse phase HPLC or quantitative LC-MS/MS.

NE18. The composition according to any one of the preceding numbered embodiments, wherein the concentration of nAra h 1, nAra h 2, nAra h 3 and nAra h 6 in option f) is determined by analytical scale reverse phase HPLC comprising separation of nAra h 1, nAra h 2, nAra h 3, and nAra h 6 by use of mixed isocratric and gradient elution, which gradient elution comprises mixing an eluent A consisting of aqueous 0.1% trifluoroacetic acid with increasing volumes of an eluent B consisting of acetonitrile mixed with 0.1% trifluoroacetic acid, quantification may be performed against pure calibration standards of nAra h 1, nAra h 2, nAra h 3, and nAra h 6, optionally converting the concentration nAra h 1, nAra h 2, nAra h 3 and nAra h 6 by weight in the composition to molar concentrations by using the molar mass of 68757 g/mol for Ara h 1, the molar mass of 17994 g/mol for Ara h 2, the molar mass of 58600 g/mol for Ara h 3 and the molar mass of 14846 g/mol for Ara h 6.

NE19. The composition according to any one of the preceding numbered embodiments, wherein the concentration by weight of the total mass of peanut proteins in the composition of each of nAra h 1, nAra h 2, nAra h 3 and nAra h 6 in option f) is in the range of 20% to 60% for nAra h 1; in the range of 5% to 15% for nAra h2 (optionally 4% to 20%; in the range of 15% to 50% (optionally in the range of 20 to 60%; for nAra h 3, in the range of 4% to 12% (optionally in the range of 4-18%) for nAra h 6, provided that the sum of nAra h 1, nAra h 2, nAra 3 and nAra 6 at least constitute 75% by weight of total peanut protein, or wherein the concentration by weight of the total mass of peanut proteins for nAra h 1 is in the range of 20% to 60%; for nAra h 2 it is in the range of 4% to 20%; for nAra h3 it is in the range of 20% to 60% and for nAra h 6 it is in the range of 4% to 18%, provided that the sum of nAra h 1, nAra h 2, nAra 3 and nAra 6 constitute at least 75% by weight of total peanut protein, OR wherein the concentration by weight of the total mass of peanut proteins for nAra h 1 is in the range of 25% to 60%; for nAra h2 it is in the range of 6% to 14%; for nAra h3 it is in the range of 20% to 55% and for nAra h 6 it is the range of 5% to 15%, provided that the sum of nAra h 1, nAra h 2, nAra 3 and nAra 6 at least constitute 75% by weight of total peanut protein.

NE20. The composition according to any one of the preceding numbered embodiments, wherein the concentration by weight of the total mass of peanut proteins in the composition of each of nAra h 1, nAra h 2, nAra h 3 and nAra h 6 in option f) is in the range of 21% to 53% for nAra h 1, in the range of 5.5% to 14% for nAra h2, in the range of 18% to 46% for nAra h 3, in the range of 5% to 11% for nAra h 6, provided that the sum of nAra h 1, nAra h 2, nAra 3 and nAra 6 at least constitutes 75% by weight of total peanut protein.

NE21. The composition according to any one of the preceding numbered embodiments, wherein the controlled concentration by weight of the total mass of peanut proteins in the composition of each of nAra h 1, nAra h 2, nAra h 3 and nAra h 6 in option f) is in the range of 25% to 50% for nAra h 1, in the range of 6.5% to 13% for nAra h2, in the range of 21% to 42% for nAra h 3, in the range of 5% to 11% for nAra h 6, provided that the sum of nAra h 1, nAra h 2, nAra 3 and nAra 6 at least constitutes 75% by weight of total peanut protein.

NE22. The composition according to any one of the preceding numbered embodiments, preferably to any one of numbered embodiments NE19 to NE21, wherein the sum of nAra h 1, nAra h 2, nAra 3 and nAra 6 constitute at least 75% by weight of the total peanut protein in the composition, such as at least 80%, such as at least 85%, such as at least 90%, typically wherein nAra h 1, nAra h 2, nAra h 3, and nAra h 6 in combination constitute at the most 98%, 99% or 100% by weight of the total peanut protein in the composition of the, such that nAra h 1, nAra h 2, nAra h 3, and nAra h 6 in combination constitute in the range of 75% to 99% by weight of total peanut protein, preferably in the range of 75% to 98%, such as in the range of 80% to 100%, 80% to 99%, 80% to 98%, such as in the range of 85% to 100%, 85% to 99%, 85% to 98%.

NE23. The composition according to any one of the preceding numbered embodiments, wherein the concentration, such as a controlled concentration, of each of nAra h 1, nAra h 3, and nAra h 6, and optionally each of nAra h 1, nAra h 2, nAra h 3 and nAra h 6, in options e) and Error!Reference source not found.) is in the range of 2-12 nmol/mg of the total mass of peanut proteins in the composition, such as in the range of 3-11 nmol/mg, such as in the range of 3-10 nmol/mg, such as in the range of 3-9 nmol/mg, such as in the range of 2.8 nmol/mg to 8.4 nmol/mg of the total mass of peanut proteins in the composition. Optionally the molar ratio of each of the pairs nAra h 1:nAra h 2, nAra h 3:nAra h 2, nAra h 6:nAra h 2 is in the range of 0.5-2.0 such as in the range of 0.5 to 1.5. Optionally wherein the concentration is controlled/determined by analytical scale RP-HPLC or quantitative immune assay.

NE24. The composition according to any one of the preceding numbered embodiments, wherein the concentration, such as a controlled concentration, of each of nAra h 1, nAra h 3 and nAra h 6, and optionally each of nAra h 1, nAra h 2, nAra h 3 and nAra h 6, in options e) and Error!Reference source not found.) is in the range of 3.1 nmol/mg to 7.8 nmol/mg of the total mass of peanut proteins in the composition. Optionally the molar ratio of each of the pairs nAra h 1:nAra h 2, nAra h 3:nAra h 2, nAra h 6:nAra h 2 is in the range of 0.5 to 2.0, such as in the range of 0.5 to 1.5. Optionally wherein the concentration is controlled/determined by analytical scale RP-HPLC or quantitative immune assay.

NE25. The composition according to any one of the preceding numbered embodiments, wherein the concentration, such as a controlled concentration, of each of nAra h 1, nAra h 3 and nAra h 6, and optionally each of nAra h 1, nAra h 2, nAra h 3 and nAra h 6, in options e) and Error!Reference source not found.) is in the range of 3.6 nmol/mg to 7.1 nmol/mg of the total mass of peanut proteins in the composition and provided that the molar ratio of each of the pairs nAra h 1:nAra h 2, nAra h 3:nAra h 2, nAra h 6:nAra h 2 is on the range 0.5 to 2.0, such as in the range of 0.5 to 1.5., Optionally wherein the concentration is controlled/determined by analytical scale RP-HPLC or quantitative immune assay.

NE26. The composition according to any of numbered embodiments NE23-NE25, wherein the molar ratio of each of the pairs nAra h 1:nAra h 2, nAra h 3:nAra h 2, nAra h 6:nAra h 2 is the range of 0.6 to 1.4.

NE27. The composition according to any of numbered embodiments NE23 to NE26, wherein the molar ratio of each of the pairs nAra h 1:nAra h 2, nAra h 3:nAra h 2, nAra h 6:nAra h 2 is the range of 0.7 to 1.3.

NE28. The composition according to any one of the preceding numbered embodiments, wherein the concentrations of nAra h 1, nAra h 2, nAra h 3, and nAra h 6 in the individual fractions obtained in step 2 in h) and/or i) are determined/controlled, preferably by analytical scale RP-HPLC or quantitative immune assay.

NE29. The composition according to any one of the preceding numbered embodiments, wherein the fractions obtained in step 2 in option(s) h) and/or i) are enriched for Ara h species in the following order of elution: nAra h 6, nAra h 2, nAra h 1 and nAra h 3.

NE30. The composition according to any one of the preceding numbered embodiments, wherein the fractions or aliquots thereof in step 5 of option h) and/or in step 4 of option i) is combined to produce a molar ratio of each of the pairs nAra h 1:nAra h 2, nAra h 3:nAra h 2, nAra h 6:nAra h 2 in the range of 0.5 to 2.0, such as in the range of 0.5 to 1.8, such as in the range of 0.5 to 1.5. preferably in the range of 0.6 to 1.4, such as in the range of 0.7 to 1.3.

NE31. The composition according to any one of the preceding numbered embodiments, wherein the molar ratio of option j) of each of the pairs nAra h 1:nAra h 2, nAra h 3:nAra h 2, nAra h 6:nAra h 2 is in the range of 0.5 to 2.0, such as in the range of 0.5 to 1.8, such as in the range of 0.5 to 1.5, such as in the range of 0.6 to 1.4, such as in the range of 0.7 to 1.3.

NE32. The composition according to any one of the preceding numbered embodiments, wherein each of nAra h 1, nAra h 2, nAra h 3, and nAra h 6 comprises their naturally occurring isoforms and/or naturally occurring oligomeric conformations NE33. The composition according to any one of the preceding numbered embodiments, said composition comprises the nAra h 3 in a conformation selected from the group consisting of monomeric nAra h 3, trimeric nAra h 3 and hexameric nAra h 3, such as wherein nAra h 3 is present in a mixture of monomeric, trimeric and hexameric nAra h 3, such as predominantly present as a mixture of trimeric and hexameric nAra h 3, optionally wherein nAra h 1 is present predominantly in its trimeric conformation.

NE34. The composition according to any one of the preceding numbered embodiments, which is free from aggregates comprising a nAra h 3 polypeptides and/or a nAra h 1 polypeptides, wherein the aggregates have a molecular mass ≥700 kDa.

NE35. The composition according to any one of the preceding numbered embodiments, wherein the amount of nAra h 2 is in the range of 4% to 20% by weight of the total mass of peanut proteins in the composition, such as in the range of 4 to 18%, such as in the range of 5 to 15%, 5.5 to 14%, such as in the range of 6.5% to 13%, such as in the range of 7% to 12% by weight of the total mass of peanut proteins in the composition.

NE36. The composition according to any one of the preceding numbered embodiments, wherein nAra h 1, nAra h 2, nAra h 3, and nAra h 6 in combination constitute at least 75% by weight of the total peanut protein in the composition, such as at least 80%, such as at least 90%, typically wherein nAra h 1, nAra h 2, nAra h 3, and nAra h 6 in combination constitute at the most 98%, 99% or 100% by weight of the total peanut protein in the composition of the, such that nAra h 1, nAra h 2, nAra h 3, and nAra h 6 in combination constitute from 75% to 100% by weight of the total peanut protein in the composition, such as in the range of 75% to 99% by weight of total peanut protein, preferably in the range of 75% to 98%, such as in the range of 80% to 100%, 80% to 99%, 80% to 98%, such as in the range of 85% to 100%, 85% to 99%, 85% to 98%.

NE37. The composition according to any one of the preceding numbered embodiments, wherein the total mass of peanut protein is determined by amino acid analysis (AAA) or by the Bradford protein assay using Bovine Serum Albumin as reference standard (BCA), preferably by amino acid analysis (AAA).

NE38. The composition according to any one of the preceding numbered embodiments, which further comprises a pharmaceutically acceptable carrier, diluent, excipient, or vehicle.

NE39. A pharmaceutically acceptable formulation, wherein the formulation comprises a composition according to any one of the preceding numbered embodiments dissolved or dispersed in a carrier substance selected from the group consisting of a liquid, a semi-solid, and a solid carrier substance.

NE40. The pharmaceutically acceptable formulation according to numbered embodiment NE39, which comprises a controlled amount of nAra h 2, preferably a controlled amount of each of nAra h 1, nAra h 2, nAra h 3, and nAra h 6, respectively.

NE41. The pharmaceutically acceptable formulation according to numbered embodiment NE39 or NE40, wherein the carrier is a solid carrier substance, preferably a solid carrier substance suitable for forming a sublingual solid dosage form, such as a sublingual solid unit dosage form.

NE42. The pharmaceutically acceptable formulation according to numbered embodiment NE41, wherein the solid formulation is a tablet (compressed or non-compressed), a film, a paste or lyophilizate (such as a unit dose lyophilisate), preferably a sublingual tablet, sublingual film or sublingual lyophilisate (sublingual unit dose lyophilisate).

NE43. The pharmaceutically acceptable formulation according to numbered embodiment NE41 or NE42, which is fast-dispersing when exposed to human saliva, preferably wherein the fast-dispersing solid formulation is disintegrated within 2 minutes, such as within 1.5, 1 or within 0.5 minutes following the exposure to saliva.

NE44. The pharmaceutically acceptable formulation according to any one of numbered embodiments NE39-NE43, wherein the carrier substance comprises gelatine, preferably piscine gelatine.

NE45. The pharmaceutically acceptable formulation according to any one of numbered embodiments NE39-NE44, which is a unit dose form, preferably a sublingual unit dose form.

NE46. The pharmaceutically acceptable formulation according to numbered embodiment NE45, wherein the total quantity of peanut proteins per unit dose form is in the range 0.1-5000 μg.

NE47. The pharmaceutically acceptable formulation according to numbered embodiment NE46, wherein the quantity of nAra h 2 per unit dose form is in the range from 0.01-500 μg.

NE48. The pharmaceutically acceptable formulation according to any one of numbered embodiments NE46-NE47, wherein the total quantity of peanut proteins per unit dose form is about 0.1 μg, about 0.5 μg, about 1.0 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 10.5 μg, about 11 μg, about 11.5 μg, about 12 μg, about 12.5 μg, about 13 μg, about 13.5 μg, about 14 μg, about 14.5 μg, about 15 μg, about 15.5 μg, about 16 μg, about 16.5 μg, about 17 μg, about 17.5 μg, about 18 μg, about 18.5 μg, about 19 μg, about 19.5 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, about 500 μg, about 505 μg, about 510 μg, about 515 μg, about 520 μg, about 525 μg, about 530 μg, about 535 μg, about 540 μg, about 545 μg, about 550 μg, about 555 μg, about 560 μg, about 565 μg, about 570 μg, about 575 μg, about 580 μg, about 585 μg, about 590 μg, about 595 μg, about 600 μg, about 605 μg, about 610 μg, about 615 μg, about 620 μg, about 625 μg, about 630 μg, about 635 μg, about 640 μg, about 645 μg, about 650 μg, about 655 μg, about 660 μg, about 665 μg, about 670 μg, about 675 μg, about 680 μg, about 685 μg, about 690 μg, about 695 μg, about 700 μg, about 705 μg, about 710 μg, about 715 μg, about 720 μg, about 725 μg, about 730 μg, about 735 μg, about 740 μg, about 745 μg, about 750 μg, about 755 μg, about 760 μg, about 765 μg, about 770 μg, about 775 μg, about 780 μg, about 785 μg, about 790 μg, about 795 μg, about 800 μg, about 805 μg, about 810 μg, about 815 μg, about 820 μg, about 825 μg, about 830 μg, about 835 μg, about 840 μg, about 845 μg, about 850 μg, about 855 μg, about 860 μg, about 865 μg, about 870 μg, about 875 μg, about 880 μg, about 885 μg, about 890 μg, about 895 μg, about 900 μg, about 905 μg, about 910 μg, about 915 μg, about 920 μg, about 925 μg, about 930 μg, about 935 μg, about 940 μg, about 945 μg, about 950 μg, about 955 μg, about 960 μg, about 965 μg, about 970 μg, about 975 μg, about 980 μg, about 985 μg, about 990 μg, about 995 μg, about 1000 μg, about 1050 μg, about 1100 μg, about 1150 μg, about 1200 μg, about 1250 μg, about 1300 μg, about 1350 μg, about 1400 μg, about 1450 μg, about 1500 μg, about 1550 μg, about 1600 μg, about 1650 μg, about 1700 μg, about 1750 μg, about 1800 μg, about 1850 μg, about 1900 μg, about 1950 μg, about 2000 μg, about 2050 μg, about 2100 μg, about 2150 μg, about 2200 μg, about 2250 μg, about 2300 μg, about 2350 μg, about 2400 μg, about 2450 μg, about 2500 μg, about 2550 μg, about 2600 μg, about 2650 μg, about 2700 μg, about 2750 μg, about 2800 μg, about 2850 μg, about 2900 μg, about 2950 μg, about 3000 μg, about 3050 μg, about 3100 μg, about 3150 μg, about 3200 μg, about 3250 μg, about 3300 μg, about 3350 μg, about 3400 μg, about 3450 μg, about 3500 μg, about 3550 μg, about 3600 μg, about 3650 μg, about 3700 μg, about 3750 μg, about 3800 μg, about 3850 μg, about 3900 μg, about 3950 μg, about 4000 μg, about 4050 μg, about 4100 μg, about 4150 μg, about 4200 μg, about 4250 μg, about 4300 μg, about 4350 μg, about 4400 μg, about 4450 μg, about 4500 μg, about 4550 μg, about 4600 μg, about 4650 μg, about 4700 μg, about 4750 μg, about 4800 μg, about 4850 μg, about 4900 μg, about 4950 μg, or about 5000 μg.

NE49. The pharmaceutically acceptable formulation according to any one of numbered embodiments NE46-NE48, wherein the amount of Ara h 2 is about 0.01 μg, about 0.05 μg, about 0.1 μg, about 0.15 μg, about 0.2 μg, about 0.25 μg, about 0.3 μg, about 0.35 μg, about 0.4 μg, about 0.45 μg, about 0.5 μg, about 0.55 μg, about 0.6 μg, about 0.65 μg, about 0.7 μg, about 0.75 μg, about 0.8 μg, about 0.85 μg, about 0.9 μg, about 0.95 μg, about 1.0 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 10.5 μg, about 11 μg, about 11.5 μg, about 12 μg, about 12.5 μg, about 13 μg, about 13.5 μg, about 14 μg, about 14.5 μg, about 15 μg, about 15.5 μg, about 16 μg, about 16.5 μg, about 17 μg, about 17.5 μg, about 18 μg, about 18.5 μg, about 19 μg, about 19.5 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, or about 500 μg.

NE50. The pharmaceutically acceptable formulation according to any one of numbered embodiments NE39-NE49, wherein the quantities of nAra h 1, nAra h 2, nAra h 3, and nAra h 6 are as defined in any one of numbered embodiments NE1-NE38.

NE51. The pharmaceutically acceptable formulation according to any one of numbered embodiments NE39-NE50, which comprises the composition according to any one of numbered embodiments NE1-NE38.

NE52. The composition according to any one of numbered embodiments NE1-NE38 or the pharmaceutically acceptable formulation according to any one of numbered embodiments NE39-NE51 for use as a medicament.

NE53. The composition according to any one of numbered embodiments NE1-NE38 or the pharmaceutically acceptable formulation according to any one of numbered embodiments NE39-NE51 for use in a method of treating a human against peanut allergy, optionally wherein the treating of peanut allergy comprises allergen-specific immunotherapy.

NE54. The composition or pharmaceutical formulation for the use according to numbered embodiment NE53 wherein allergen-specific immunotherapy comprises a plurality of administrations of the composition or formulation.

NE55. The composition or pharmaceutical formulation for the use according to numbered embodiment NE54, wherein the plurality of administrations is a plurality of administrations separated by at least one day, and where the plurality of administration preferably is in the form of one daily dose, such as one daily administration of a dose.

NE56. The composition or pharmaceutical formulation for the use according to any one of numbered embodiments NE53-NE55, wherein allergen-specific immunotherapy comprises administration of a plurality of identical daily doses of peanut protein, optionally preceded by a plurality of consecutive non-identical daily doses.

NE57. The composition or formulation for the use according to any one of numbered embodiments NE53-NE56, wherein allergen-specific immunotherapy comprises administration of a plurality of consecutive non-identical daily doses of peanut protein, optionally preceding a plurality of identical daily doses.

NE58. The composition or formulation for the use according to any one of numbered embodiments NE56-NE57, wherein the plurality of consecutive non-identical daily doses is in the form of consecutive daily doses where no earlier dose is higher than a later dose.

NE59. The composition or formulation for the use according to numbered embodiment NE58, wherein each daily non-identical dose is higher than any preceding dose in the series.

NE60. The composition or formulation for the use according to any one of numbered embodiments NE56-NE59, wherein the number of daily non-identical doses is selected from 2, 3, 4, 5, 6, 7, 8, 9, 10, 11, 12, 13, 14, 15, 16, 17, 18, 19, 20, 21, 22, 23, 24, 25, 26, 27, 28, 29, and 30 consecutive non-identical daily non-identical doses.

NE61. The composition or formulation for the use according to any one of numbered embodiments NE53-NE60, wherein the lowest total daily dose of peanut protein is 0.1 μg, and where the highest total daily dose is 5000 μg.

NE62. The composition or formulation for the use according to any one of numbered embodiments 56-62, wherein the lowest daily dose is between 0.1 μg and 200 μg of peanut protein, such as about 0.1 μg, about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, and about 200 μg.

NE63. The composition or formulation for the use according to any one of numbered embodiments NE53-NE62, wherein the highest daily dose is between 300 and 5,000 μg of peanut protein, such as about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 820 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1000 μg, about 1010 μg, about 1020 μg, about 1030 μg, about 1040 μg, about 1050 μg, about 1060 μg, about 1070 μg, about 1080 μg, about 1090 μg, about 1100 μg, about 1110 μg, about 1120 μg, about 1130 μg, about 1140 μg, about 1150 μg, about 1160 μg, about 1170 μg, about 1180 μg, about 1190 μg, about 1200 μg, about 1210 μg, about 1220 μg, about 1230 μg, about 1240 μg, about 1250 μg, about 1260 μg, about 1270 μg, about 1280 μg, about 1290 μg, about 1300 μg, about 1310 μg, about 1320 μg, about 1330 μg, about 1340 μg, about 1350 μg, about 1360 μg, about 1370 μg, about 1380 μg, about 1390 μg, about 1400 μg, about 1410 μg, about 1420 μg, about 1430 μg, about 1440 μg, about 1450 μg, about 1460 μg, about 1470 μg, about 1480 μg, about 1490 μg, about 1500 μg, about 1510 μg, about 1520 μg, about 1530 μg, about 1540 μg, about 1550 μg, about 1560 μg, about 1570 μg, about 1580 μg, about 1590 μg, about 1600 μg, about 1610 μg, about 1620 μg, about 1630 μg, about 1640 μg, about 1650 μg, about 1660 μg, about 1670 μg, about 1680 μg, about 1690 μg, about 1700 μg, about 1710 μg, about 1720 μg, about 1730 μg, about 1740 μg, about 1750 μg, about 1760 μg, about 1770 μg, about 1780 μg, about 1790 μg, about 1800 μg, about 1810 μg, about 1820 μg, about 1830 μg, about 1840 μg, about 1850 μg, about 1860 μg, about 1870 μg, about 1880 μg, about 1890 μg, about 1900 μg, about 1910 μg, about 1920 μg, about 1930 μg, about 1940 μg, about 1950 μg, about 1960 μg, about 1970 μg, about 1980 μg, about 1990 μg, about 2000 μg, about 2010 μg, about 2020 μg, about 2030 μg, about 2040 μg, about 2050 μg, about 2060 μg, about 2070 μg, about 2080 μg, about 2090 μg, about 2100 μg, about 2110 μg, about 2120 μg, about 2130 μg, about 2140 μg, about 2150 μg, about 2160 μg, about 2170 μg, about 2180 μg, about 2190 μg, about 2200 μg, about 2210 μg, about 2220 μg, about 2230 μg, about 2240 μg, about 2250 μg, about 2260 μg, about 2270 μg, about 2280 μg, about 2290 μg, about 2300 μg, about 2310 μg, about 2320 μg, about 2330 μg, about 2340 μg, about 2350 μg, about 2360 μg, about 2370 μg, about 2380 μg, about 2390 μg, about 2400 μg, about 2410 μg, about 2420 μg, about 2430 μg, about 2440 μg, about 2450 μg, about 2460 μg, about 2470 μg, about 2480 μg, about 2490 μg, about 2500 μg, about 2510 μg, about 2520 μg, about 2530 μg, about 2540 μg, about 2550 μg, about 2560 μg, about 2570 μg, about 2580 μg, about 2590 μg, about 2600 μg, about 2610 μg, about 2620 μg, about 2630 μg, about 2640 μg, about 2650 μg, about 2660 μg, about 2670 μg, about 2680 μg, about 2690 μg, about 2700 μg, about 2710 μg, about 2720 μg, about 2730 μg, about 2740 μg, about 2750 μg, about 2760 μg, about 2770 μg, about 2780 μg, about 2790 μg, about 2800 μg, about 2810 μg, about 2820 μg, about 2830 μg, about 2840 μg, about 2850 μg, about 2860 μg, about 2870 μg, about 2880 μg, about 2890 μg, about 2900 μg, about 2910 μg, about 2920 μg, about 2930 μg, about 2940 μg, about 2950 μg, about 2960 μg, about 2970 μg, about 2980 μg, about 2990 μg, about 3000 μg, about 3010 μg, about 3020 μg, about 3030 μg, about 3040 μg, about 3050 μg, about 3060 μg, about 3070 μg, about 3080 μg, about 3090 μg, about 3100 μg, about 3110 μg, about 3120 μg, about 3130 μg, about 3140 μg, about 3150 μg, about 3160 μg, about 3170 μg, about 3180 μg, about 3190 μg, about 3200 μg, about 3210 μg, about 3220 μg, about 3230 μg, about 3240 μg, about 3250 μg, about 3260 μg, about 3270 μg, about 3280 μg, about 3290 μg, about 3300 μg, about 3310 μg, about 3320 μg, about 3330 μg, about 3340 μg, about 3350 μg, about 3360 μg, about 3370 μg, about 3380 μg, about 3390 μg, about 3400 μg, about 3410 μg, about 3420 μg, about 3430 μg, about 3440 μg, about 3450 μg, about 3460 μg, about 3470 μg, about 3480 μg, about 3490 μg, about 3500 μg, about 3510 μg, about 3520 μg, about 3530 μg, about 3540 μg, about 3550 μg, about 3560 μg, about 3570 μg, about 3580 μg, about 3590 μg, about 3600 μg, about 3610 μg, about 3620 μg, about 3630 μg, about 3640 μg, about 3650 μg, about 3660 μg, about 3670 μg, about 3680 μg, about 3690 μg, about 3700 μg, about 3710 μg, about 3720 μg, about 3730 μg, about 3740 μg, about 3750 μg, about 3760 μg, about 3770 μg, about 3780 μg, about 3790 μg, about 3800 μg, about 3810 μg, about 3820 μg, about 3830 μg, about 3840 μg, about 3850 μg, about 3860 μg, about 3870 μg, about 3880 μg, about 3890 μg, about 3900 μg, about 3910 μg, about 3920 μg, about 3930 μg, about 3940 μg, about 3950 μg, about 3960 μg, about 3970 μg, about 3980 μg, about 3990 μg, about 4000 μg, about 4010 μg, about 4020 μg, about 4030 μg, about 4040 μg, about 4050 μg, about 4060 μg, about 4070 μg, about 4080 μg, about 4090 μg, about 4100 μg, about 4110 μg, about 4120 μg, about 4130 μg, about 4140 μg, about 4150 μg, about 4160 μg, about 4170 μg, about 4180 μg, about 4190 μg, about 4200 μg, about 4210 μg, about 4220 μg, about 4230 μg, about 4240 μg, about 4250 μg, about 4260 μg, about 4270 μg, about 4280 μg, about 4290 μg, about 4300 μg, about 4310 μg, about 4320 μg, about 4330 μg, about 4340 μg, about 4350 μg, about 4360 μg, about 4370 μg, about 4380 μg, about 4390 μg, about 4400 μg, about 4410 μg, about 4420 μg, about 4430 μg, about 4440 μg, about 4450 μg, about 4460 μg, about 4470 μg, about 4480 μg, about 4490 μg, about 4500 μg, about 4510 μg, about 4520 μg, about 4530 μg, about 4540 μg, about 4550 μg, about 4560 μg, about 4570 μg, about 4580 μg, about 4590 μg, about 4600 μg, about 4610 μg, about 4620 μg, about 4630 μg, about 4640 μg, about 4650 μg, about 4660 μg, about 4670 μg, about 4680 μg, about 4690 μg, about 4700 μg, about 4710 μg, about 4720 μg, about 4730 μg, about 4740 μg, about 4750 μg, about 4760 μg, about 4770 μg, about 4780 μg, about 4790 μg, about 4800 μg, about 4810 μg, about 4820 μg, about 4830 μg, about 4840 μg, about 4850 μg, about 4860 μg, about 4870 μg, about 4880 μg, about 4890 μg, about 4900 μg, about 4910 μg, about 4920 μg, about 4930 μg, about 4940 μg, about 4950 μg, about 4960 μg, about 4970 μg, about 4980 μg, about 4990 μg, and about 5000 μg.

NE64. The composition or pharmaceutical formulation for the use according to any one of numbered embodiments NE53-NE63, which comprises administration of one first series of a plurality of identical daily doses which precedes at least one further series of a plurality of identical daily doses, which are different than the daily doses in the first series, and which preferably are higher than the daily doses in the first series.

NE65. The composition or pharmaceutical formulation for the use according to numbered embodiment NE64, where a plurality of series of identical daily doses are administered as an updosing phase of the allergen-specific immunotherapy, wherein the daily dose in a series of identical daily doses is higher than the daily dose in any preceding series of identical daily doses.

NE66. The composition or pharmaceutical formulation for the use according to numbered embodiment NE65, wherein the plurality of series is selected from 2, 3, 4, 5, 6, 7, 8, 9, and 10 series.

NE67. The composition or pharmaceutical formulation for use according to any one of numbered embodiments NE64-NE66, wherein a series has a duration in the range of 6 to 30 days, such as in the range of 6 to 22 days, for instance in the range of 6 to 16 days, and preferably about 14 days.

NE68. The composition of pharmaceutical formulation for use according to any one of numbered embodiments NE64 to NE67, wherein—after completion of the updosing phase—the allergen-specific immunotherapy is continued with a maintenance phase comprising administering a plurality of daily doses which are identical with the daily dose of the last series in the updosing phase or is in the range of ½ to 9/10 of the daily dose of the last series in the updosing phase.

NE69. The composition of pharmaceutical formulation for use according to any one of numbered embodiments NE53 to NE68, wherein the allergen-specific immunotherapy comprises administration to the oral mucosa, preferably by sublingual administration.

NE70. A method for preparing a composition comprising two or more of nAra h 1, nAra h 2, nAra h 3, and nAra h 6, the method comprising providing 1) an extract of peanut protein obtained by extracting raw peanut kernels with an aqueous solvent to obtain an aqueous extract comprising each of nAra h 1, nAra h 2, nAra h 3, and nAra h 6 and 2) subjecting said aqueous extract to anion exchange chromatography with stepwise or continuous aqueous salt gradient elution at pH in the range of 7 to 9, whereby each of nAra h 1, nAra h 2, nAra h 3, and nAra h 6 are eluted and collected into fractions individually enriched for nAra h 1, nAra h 2, nAra h 3, or nAra h 6; 3) optionally collecting a flow-through fraction from the anion exchange chromatography; and 4) combining two or more fractions or aliquots thereof as obtained in step 2 or in combined step 2 and 3 to obtain said peanut protein composition comprising at least two of nAra h 1, nAra h 2, nAra h 3, and nAra h 6. Preferably, fractions containing peanut protein with high molecular mass have been discarded.

NE71. The method according to numbered embodiment NE70, wherein four fractions individually enriched with either nAra h 1, nAra h 2, nAra h 3, and nAra h 6 are collected and wherein combining the four fractions or aliquots thereof provide a composition comprising each of nAra h 1, nAra h 2, nAra h 3, and nAra h 6.

NE72. The method according to numbered embodiments NE70 or NE71, wherein the solution is obtained by extraction of peanut protein from raw peanut kernels with a buffered aqueous solvent having pH in the range of 6 to 9, optionally with a buffered saline aqueous solvent having pH in the range of 6 to 9.

NE73. The method according to numbered embodiment NE72, wherein the pH of the buffered aqueous solvent is in the range of 6.5 to 8.5, such as in the range of 6.5 to 9, such as in the range of 6.5 to 8, such as in the range of 7 to 9, such as in the range of 7 to 8.5, such as in the range of 7 to 8.5, and preferably in the range of 7 to 8.

NE74. The method according to numbered embodiments NE70 to NE73, wherein fractioning of the solution by anion exchange chromatography comprises stepwise or continuous aqueous salt gradient elution at a pH in the range of 7 to 9, whereby nAra h 1, nAra h 2, nAra h 3, and nAra h 6 are eluted and collected into the at least two individual fractions, and wherein the pH preferably is in the range of 7 to 8.5, preferably in the range of 7 to 8, such as in the range of 7.2 to 7.8.

NE75. The method according to numbered embodiment NE70 to NE74, wherein the concentrations of the four allergens nAra h 1, nAra h 2, nAra h 3, and nAra h 6 in the at least two fractions are quantified by RP-HPLC in order to combine aliquots of the at least two fractions to obtain the two or more peanut allergens in a composition containing controlled amounts or pre-selected amounts of two or more of the four allergens.

NE76. The method according to numbered embodiments NE70 to NE75, wherein the process comprising the steps of:
1) extracting peanut protein from raw peanut kernels with an aqueous solvent;
2) subjecting the extracted protein to anion exchange chromatography with stepwise or continuous aqueous salt gradient elution at a pH in the range of 7 to 9, whereby each of nAra h 1, nAra h 2, nAra h 3, and nAra h 6 are eluted and collected into individual fractions;
3) optionally collecting a flow-through fraction from the anion exchange chromatography; and
4) combining the entire fractions or aliquots of each fraction obtained in step 2 and optionally step 3, to obtain a composition comprising each of nAra h 1, nAra h 2, nAra h 3, and nAra h 6.

NE77. The method according to numbered embodiment NE76, wherein the concentrations of the four allergens nAra h 1, nAra h 2, nAra h 3, and nAra h 6 in the fractions of step 2 are quantified by RP-HPLC in order to combine aliquots of the fractions to obtain the four peanut allergens in a composition containing controlled amounts or pre-selected amounts of the four allergens.

NE78. The method according to numbered embodiments NE70 to NE77, wherein the fractions or aliquots thereof are combined to obtain a composition comprising each of nAra h 1, nAra h 2, nAra h 3, nAra h 6 in the composition and provided that the molar ratio of each of the pairs nAra h 1:nAra h 2, nAra h 3:nAra h 2, nAra h 6:nAra h 2 are in the range of 0.5 to 1.5 when determined by analytical scale RP-HPLC, preferably the molar ratio of each of the pairs are in the range of 0.6 to 1.4, such as in the range of 0.7 to 1.3.

NE79. The method according to numbered embodiments NE70 to NE78, wherein fractions containing peanut protein with high molecular mass have been discarded by discarding fractions eluting later than the enriched fractions of nAra h 1, nAra h 2, nAra h 3, and nAra h 6.

NE80. The method according to any one of numbered embodiments NE72 to NE79, wherein the aqueous solvent comprises TRIS in a molar range of 10 to 200 mM, preferably in the range of 10 to 100 mM, such as in the range of 10 to 50 mM, and optionally comprises NaCl or an equivalent salt in an amount in the range of 5 to 200 mM, preferably in the range of 10 to 100 mM, 10 to 50 mM.

NE81. The method according to any one of numbered embodiments NE74 to NE80, wherein the stepwise or continuous aqueous salt gradient elution is carried out using NaCl as the salt or a salt equivalent to NaCl.

NE82. A kit comprising a sealed package comprising a plurality of separate compartments, each compartment comprising a unit dose form of the pharmaceutically acceptable formulation according to any one of numbered embodiments NE41-NE51, wherein at least one unit dose form comprises an amount of total peanut allergen, which is non-identical with the amount in another unit dose form in the kit.

NE83. The kit according to numbered embodiment NE82, wherein at least one dose is unique, and wherein preferably no unit doses are identical.

NE84. The kit according to numbered embodiment NE82, wherein a first plurality of unit doses are identical, and wherein at least one further plurality of unit doses are identical but higher than the unit doses in the first plurality of unit doses.

NE85. The kit according to numbered embodiment NE84, which comprises at least 3 pluralities of identical unit doses, each comprising unit doses that are different from the unit dose in any of the other pluralities of unit doses.

NE86. The kit according to numbered embodiment NE85, wherein the at least 3 pluralities is selected from 3, 4, 5, 6, 7, 8, 9, 10, 11, 12, 13, 14, and 15.

NE87. The kit according to numbered embodiments 83 to 87, wherein the quantity of peanut protein in each unit dose form is as defined for the daily doses in any one of numbered embodiments NE61-NE63.

NE88. A method of treating a human against peanut allergy, such as mitigation of peanut allergy and/or peanut allergen-induced anaphylaxis, such as conducting peanut allergen-specific immunotherapy, the method comprising administration of at most one daily dose of a composition according to any one of numbered embodiments NE1-NE38 or the pharmaceutically acceptable formulation according to any one of numbered embodiments NE39-NE53 over a prolonged period of time.

NE89. The method according to numbered embodiment NE88, wherein the administration is to the oral mucosa, such as to the sublingual mucosa.

NE90. The method according to numbered embodiment NE88 or NE89 which comprises multiple daily administrations of non-identical doses of peanut allergen, optionally preceding multiple daily administrations of identical daily doses of peanut allergen.

NE91. The method according to any one of numbered embodiments NE88-NE90, which comprises multiple daily administrations of identical doses of peanut allergen, optionally preceded by multiple daily administrations of non-identical doses of peanut allergen.

NE92. The method according to numbered embodiment NE91, which comprises administrations of a plurality of series of identical daily doses of peanut allergen, wherein the daily doses in at least one series are non-identical with the daily doses in another series.

NE93. The method according to numbered embodiment NE92, wherein each of the plurality of series comprises daily doses that are different from the doses in any other of the series in the plurality of series, and wherein each series following an earlier series comprises higher doses than the earlier series.

NE94. The method according to any one of numbered embodiments NE92-NE93, wherein the plurality of series is constituted by 2, 3, 4, 5, 6, 7, 8, 9, 10, 11, 12, 13, 14, or 15 series.

NE95. A method of treating a human against peanut allergy, such as for mitigation of peanut allergy and/or peanut allergen-induced anaphylaxis in a human individual, such as by allergen-specific immunotherapy, the method comprising an updosing phase and optionally a maintenance phase, wherein the updosing phase comprises multiple consecutive series of administering a daily dose of peanut protein composition to the oral mucosa, wherein the daily dose within each series is identical and wherein any dose in a preceding series is lower than in a subsequent series and wherein each series has a duration ranging from 6 to 30 days; and wherein
the daily dose administered in the first series contains a total amount of peanut protein in the range of 0.1 μg to 200 μg;
the daily dose of the last series contains a total amount of peanut proteins in the range of 300 μg to 5000 μg; and
wherein the number of series is in the range from 2 to 9, such as in the range of 3 to 7, such as particularly, 3, 4, 5, 6, 7, 8, or 9, preferably 3, 4, or 5;
preferably wherein the peanut protein is extracted or extractable from raw peanut kernels by an aqueous solvent and comprises each of nAra h 1, nAra h 2, nAra h 3, and nAra h 6.

NE96. The method according to any one of numbered embodiments NE88-NE95, wherein the molar concentration ratios of each of the pairs nAra h 1:nAra h 2, nAra h 3:nAra h 2, and nAra h 6:nAra h 2 is in the range 0.5-2.0, such as in the range of 0.5 to 1.5 or more narrow for example in the range of 0.6 to 1.4, such as 0.7 to 1.3. The molar concentration for each of nAra h 1, nAra h 2, nAra h 3, and nAra h 6 is expressed as the concentration of the monomer polypeptide conformation of each of said allergens.

NE97. The method according to any one of numbered embodiments NE88-NE96 wherein the peanut protein is being essentially free from peanut protein having a molecular mass of at least 700 kDa.

NE98. The method according to any one of numbered embodiments NE88-NE97, wherein the peanut proteins comprises an amount of nAra h 2 ranging from 50-150 μg per mg peanut protein.

NE99. The method according to any one of numbered embodiments NE88-NE98, wherein the peanut protein comprises an amount of nAra h 3 ranging from 160-500 μg per mg peanut.

NE100. The method according to any one of numbered embodiments NE88-NE99, wherein nAra h 1, nAra h 2, nAra h 3, and nAra h6 in combination constitutes at least 75% by weight of the peanut protein.

NE101. The method according to any one of numbered embodiments NE88-NE100 wherein the peanut protein composition is a composition according to any one of numbered embodiments 1-NE38 or a pharmaceutically acceptable formulation according to any one of numbered embodiments NE39-NE51.

NE102. The method according to any one of numbered embodiments NE88-NE101, wherein, if series of identical doses are administered, the series each have a duration of 10-21 days, preferably about 14 days.

NE103. The method according to any one of numbered embodiments NE88-NE102, wherein, if series of identical doses are administered, the daily doses in the first series are as defined in numbered embodiment NE62.

NE104. The method according to numbered embodiment NE103, wherein the daily doses in the last series are as defined in numbered embodiment NE63.

NE105. The method according to any one of numbered embodiments NE88-NE104, wherein, if series of identical doses are administered, the daily dose of a series later than the first series is increased by a factor of 2 to 4 compared to the daily dose of the directly preceding series, such as a factor between 3 to 3.5, such a as between 2 to 3.

NE106. The method according to numbered embodiment NE105, wherein the daily dose of the first series is about 1 μg and the daily dose of the last series is about 4320 μg, and the number of series is 9, the doses of the 7 series between the first and last series are in escalating order about 3 μg, about 10 μg, about 40 μg, about 120 μg, about 360 μg, about 1080 μg, and about 2160 μg, respectively.

NE107. The method according to numbered embodiment NE105, wherein the daily dose of the first series is about 3 μg and the daily dose of the last series is about 4320 μg, and the number of series is 8, the doses of the 6 series between the first and last series are in escalating order about 10 μg, about 40 μg, about 120 μg, about 360 μg, about 1080 μg and about 2160 μg, respectively.

NE108. The method according to numbered embodiment NE105, wherein the daily dose of the first series is about 10 μg and the daily dose of the last series is about 4320 μg, and the number of series is 7, the doses of the 5 series between the first and last series are in escalating order about 40 μg, about 120 μg, about 360 μg, about 1080 μg and about 2160 μg, respectively.

NE109. The method according to numbered embodiment NE105, wherein the daily dose of the first series is about 40 μg and the daily dose of the last series is about 4320 μg, and the number of series is 6, the doses of the 4 series between the first and last series are in escalating order about 120 μg, about 360 μg, about 1080 μg and about 2160 μg, respectively.

NE110. The method according to numbered embodiment NE105, wherein the daily dose of the first series is about 120 μg and the daily dose of the last series is about 4320 μg, and the number of series is 5, the doses of the 3 series between the first and last series are in escalating order about 360 μg, about 1080 μg and about 2160 μg, respectively.

NE111. The method according to numbered embodiment NE105, wherein the daily dose of the first series is about 1 μg and the daily dose of the last series is about 2160 μg, and the number of series is 8, the doses of the 6 series between the first and last series are in escalating order about 3 μg, about 10 μg, about 40 μg, about 120 μg, about 360 μg and about 1080 μg, respectively.

NE112. The method according to numbered embodiment NE105, wherein the daily dose of the first series is about 3 μg and the daily dose of the last series is about 2160 μg, and the number of series is 7, the doses of the 5 series between the first and last series are in escalating order about 10 μg, about 40 μg, about 120 μg, about 360 μg and about 1080 μg, respectively.

NE113. The method according to numbered embodiment NE105, wherein the daily dose of the first series is about 10 μg and the daily dose of the last series is about 2160 μg, and the number of series is 6, the doses of the 4 series between the first and last series are in escalating order about 40 μg, about 120 μg, about 360 μg and about 1080 μg, respectively.

NE114. The method according to numbered embodiment NE105, wherein the daily dose of the first series is about 40 μg and the daily dose of the last series is about 2160 μg, and the number of series is 5, the doses of the 3 series between the first and last series are in escalating order about 120 μg, about 360 μg and about 1080 μg, respectively.

NE115. The method according to numbered embodiment NE105, wherein the daily dose of the first series is about 120 μg and the daily dose of the last series is about 2160 μg, and the number of series is 4, the doses of the 2 series between the first and last series are in escalating order about 360 μg and about 1080 μg, respectively.

NE116. The method according to numbered embodiment NE105, wherein the daily dose of the first series is about 1 μg and the daily dose of the last series is about 1080 μg, and the number of series is 7, the doses of the 5 series between the first and last series are in escalating order about 3 μg, about 10 μg, about 40 μg, about 120 μg and about 360 μg, respectively.

NE117. The method according to numbered embodiment NE105, wherein the daily dose of the first series is about 3 μg and the daily dose of the last series is about 1080 μg, and the number of series is 6, the doses of the 4 series between the first and last series are in escalating order about 10 μg, about 40 μg, about 120 μg and about 360 μg, respectively.

NE118. The method according to numbered embodiment NE105, wherein the daily dose of the first series is about 10 μg and the daily dose of the last series is about 1080 μg, and the number of series is 5, the doses of the 3 series between the first and last series are in escalating order about 40 μg, about 120 μg and about 360 μg, respectively.

NE119. The method according to numbered embodiment NE105, wherein the daily dose of the first series is about 40 μg and the daily dose of the last series is about 1080 μg, and the number of series is 4, the doses of the 2 series between the first and last series are in escalating order about 120 μg and about 360 μg, respectively.

NE120. The method according to numbered embodiment NE105, wherein the daily dose of the first series is about 120 μg and the daily dose of the last series is about 1080 μg, and the number of series is 3, the doses of the 1 series between the first and last series are about 360 μg.

NE121. The method according to any one of numbered embodiments NE88-NE120, wherein administration to the oral mucosa is by buccal or sublingual administration, preferably sublingual administration.

NE122. The method according to any one of numbered embodiments NE88-NE121, comprising a maintenance phase, which comprises a plurality of administrations of peanut protein doses to the oral mucosa, preferably the sublingual mucosa, with at least one day apart.

NE123. The method according to numbered embodiment NE122, wherein the dose of total protein in the maintenance phase is identical to the daily dose of any last series of administrations or is in the range of 0.5 to 0.9 of the daily dose of any last series.

NE124. The method according to any one of numbered embodiments NE88-NE123, wherein the human after completion of the updosing phase is able to tolerize at least 300 mg peanut protein in an oral food challenge test, such as at least 400 mg, 500 mg, 600 mg, 700 mg, 800 mg, 1000 mg, 1200 mg, 1500 mg, 2000 mg, 2500 mg, 3000 mg, 4000 mg, 5000 mg, or 6000 mg peanut protein.

NE125. The method according to any one of numbered embodiments NE88-NE124, wherein the human after completion of the updosing phase and six months of maintenance phase is able to tolerize at least 300 mg peanut protein in an oral food challenge test, such as at least 400 mg, 500 mg, 600 mg, 700 mg, 800 mg, 1000 mg, 1200 mg, 1500 mg, 2000 mg, 2500 mg, 3000 mg, 4000 mg, 5000 mg, or 6000 mg peanut protein.

NE126. The composition according to any one of numbered embodiments NE0-NE38 or the pharmaceutically acceptable formulation according to any one of numbered embodiments NE39-NE52 for use in the method according to any one of numbered embodiments NE88-NE125.

NE127. The composition or formulation for the use according to any one of numbered embodiments NE53-NE69, wherein the method of mitigating peanut allergy and/or anaphylaxis is in association with accidental exposure to peanuts, or peanut containing products.

NE128. The composition or formulation for the use according to any one of numbered embodiments NE53-NE69 and NE127, wherein the method of mitigating peanut allergy comprises induction of tolerance to one or more peanut allergens; one or more peanuts; peanut protein; or a peanut protein-containing product.

NE129. The composition or formulation for the use according to numbered embodiments NE1278, wherein induction of tolerance comprises tolerance to ingestion or exposure to at least 600 mg peanut protein, for example wherein induction of tolerance comprises tolerance to at least 600 mg peanut protein in an oral food challenge test.

### Example 1

### Methods for Controlling Allergen Profiles and Quantifying Allergens

This example relates to methods for controlling the allergen profiles and quantifying the amounts of the four key allergens in peanut compositions described in the examples below.

Reverse Phase HPLC (RP-HPLC) Method with UV Detection, Analytical Scale

RP-HPLC method was developed for controlling the allergen profiles and for quantifying the levels of each of the key peanut allergens Ara h 1, 2, 3, and 6 in the compositions. As HPLC column material was selected a reverse-phase C4 ligand in order to optimise the separation of proteins based on size, hydrophobicity and isoelectric point. In comparison to traditional C18 phases, the C4 ligand is less retentive and will minimise protein carryover, increase protein recovery, and improve peak capacity.

The following conditions were used:
Column: Waters ACQUITY UPLC BEH300 C4, 300 Å, 1.7 μm, 150×2.1 mm, operated at a temperature of 60° C.
Detection: UV absorbance at 210 nm or 280 nm.
Standards: Each of the four key peanut allergens was purified from raw peanuts of the cultivar variant Runner using multistep purification to obtain separate pure fractions of nAra h 1, nAra h 2, nAra h 3 and nAra h 6 as described below.
Elution: A mixed isocratic and gradient elution was applied using two mobile phases A (water with 0.1% trifluoroacetic acid) and B (acetonitrile with 0.1% trifluoroacetic acid).
   Two different elution profiles were used: the long run (86 min) or the short run (29 min). The long run elution was carried out by initial gradient mixing from 2-19% B with flow rate 0.4 ml/min to elute compounds binding weakly to the column. These are eluted isocratic at 19% B. Then the flow rate was decreased to 0.1 ml/min and a gradient from 19-32% B with 1% increase per minute was used to elute the Ara h 2 and Ara h 6 with good resolution. Then the elution was continued with increasing the percentage of eluent B by 0.1%/minute from 32-37% to elute Ara h 1 and Ara h 3. The gradient mixing with B is continued to quickly arrive at 98% B to ensure that all proteins in the sample were eluted and then to return to the initial gradient mixing with 2% B.
   The short run elution was carried out by applying a linear gradient elution from 18% B to 54% B over 18 minutes using a flow rate of 0.4 ml/min and then quickly raise the percentage of B to 98% within 0.5 min and keep the 98% B until time point 22 min. The percentage of B was then quickly dropped to 18% within 0.5 min and kept at 18% until final run time at 29 min.

### Mass Spectrometry (LC-MS/MS)

The total amount of each of the four key allergens was determined by MS quantification using specific heavy signature peptides to each of the four key allergens (AQUA peptides). The molar ratio to Ara h 2 was determined for each of the four allergens using the molecular mass of each of the allergens mentioned above. The MS method comprises digesting the allergen composition by treatment with digestive enzymes like trypsin or chymotrypsin and adding known concentrations of synthetic isotope labelled peptides to the protein digest of the extract. The concentration of the natural peptide is then determined by comparing the peak areas between the isotope labelled and natural peptides that have the same amino acid sequence but different masses. Comparison of the peak areas from the natural peptide to its isotope-labelled standard yields a ratio which is used to calculate the concentration of the natural peptide.

All mass spectrometer experiments were performed on a nanoscale HPLC system (EASY-nLC 1000 from Thermo Scientific) connected to an Orbitrap Q-Exactive equipped with a nanoelectrospray source (Thermo Fisher Scientific). Each peptide sample was autosampled and separated on a 15-cm analytical column (50 μm inner diameter) EASY-Spray™ HPLC Columns with a 1-h gradient ranging from 5 to 40% acetonitrile in 0.5% acetic acid. The effluent from the HPLC was electrosprayed directly into the mass spectrometer. The Q-Exactive mass spectrometer was operated in a targeted acquisition mode scanning only for the peptides of interest. Both full scans of the peptide ions (MS) and fragment scans of the peptide fragments ions are recorded (MS/MS scans). All raw data analyses were performed with the Skyline software suite. Concentrations were verified by spiking in known amounts of naturally purified major allergens and measuring the increase in ratio to the heavy isotope labelled peptide.

Size Exclusion HPLC (SEC) with UV Detection, Analytical Scale
Column: Yarra 1.8 μm SEC-X 300 SS, particle size 1.8 μm, pore size 300 Å, column size 150×4.6 mm, operated at a temperature of 25° C. Autosampler was operated at 20° C.
Detection: UV absorbance at various wavelengths, calculations are preferably made at 280 nm or 210 nm depending on interfering background and sample proteins.
Standards: The following standards were used as molecular mass indicators for the SEC analysis: Lyophilised mix of thyroglobulin (670 kDA), bovine γ-globulin (158 kDa), chicken ovalbumin (44 kDa), equine myoglobin (17 kDa), and vitamin B12 (1.35 kDa) obtained from BIO-RAD Gel Filtration Standard, Cat. #151-1901.
Elution: Phosphate buffered saline of 8.2 mM NaH2PO4, 137 mM NaCl, 1.7 mM KCl, and 1.5 mM KH2PO4 at pH 7.2.
Samples: A volume corresponding to Approx. 15 μg of peanut protein (determined by BCA) was applied.
   Size Exclusion Chromatography (SEC G200) with UV Detection (Preparative-Scale)

Preparative-scale SEC was carried out using a matrix well suited for high-resolution analysis and small-scale purification of monoclonal antibodies (mAb) and other biomolecules with Mr˜10 000 to ˜600 000. In the present example, a Superdex™ 200 (G200) column with the dimensions of 10×300 mm was used (supplier: Cytiva). Sample size of about 7,5 μl was applied. Aqueous phosphate-buffered saline (pH about 7.4) was used as elution solvent.

Pure Reference Standards of nAra h 1, nAra h 2, nAra h 3 and nAra h 6.

Each of the four key peanut allergens was purified from aqueous extractions of pulverized raw peanut kernels of the cultivar variant Runner (mix of genotypes) using multistep purification to obtain separate pure fractions of nAra h 1, nAra h 2, nAra h 3 and nAra h 6, which can be used as reference standards for identity or for quantification. The purified standards of the allergens were obtained by purifying a crude peanut allergen extract by combining various chromatographic methods: initially the four key allergens were separated by anion exchange chromatography, preferably by use of the strong anion exchange resin, HiTrap Q HP from Cytiva™ to separate the four key allergens into individual fractions, which were further purified by use of hydrophobic retention chromatography and size exclusion chromatography.

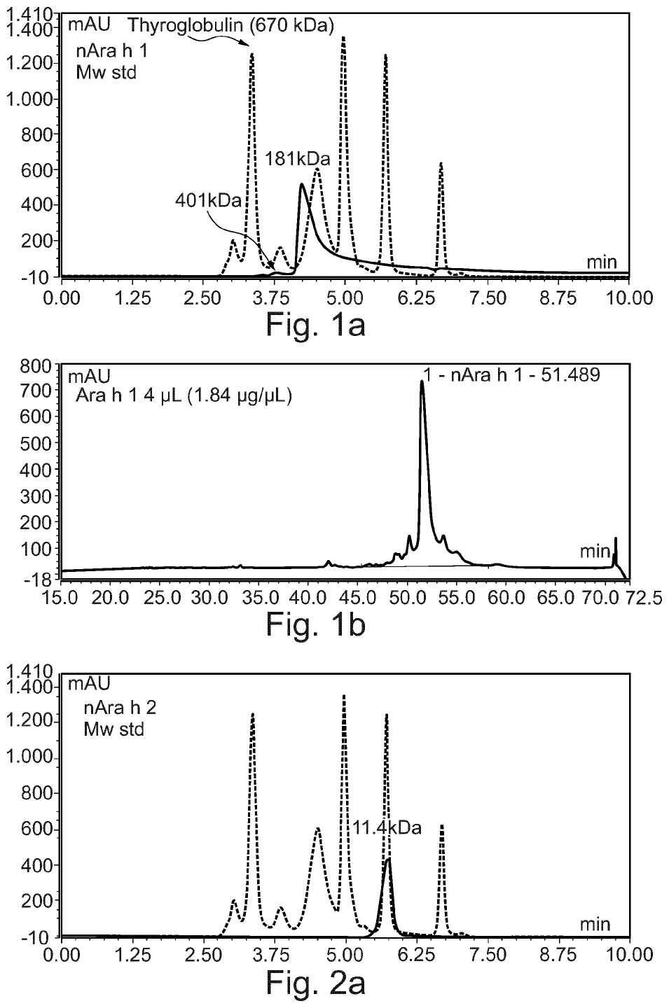

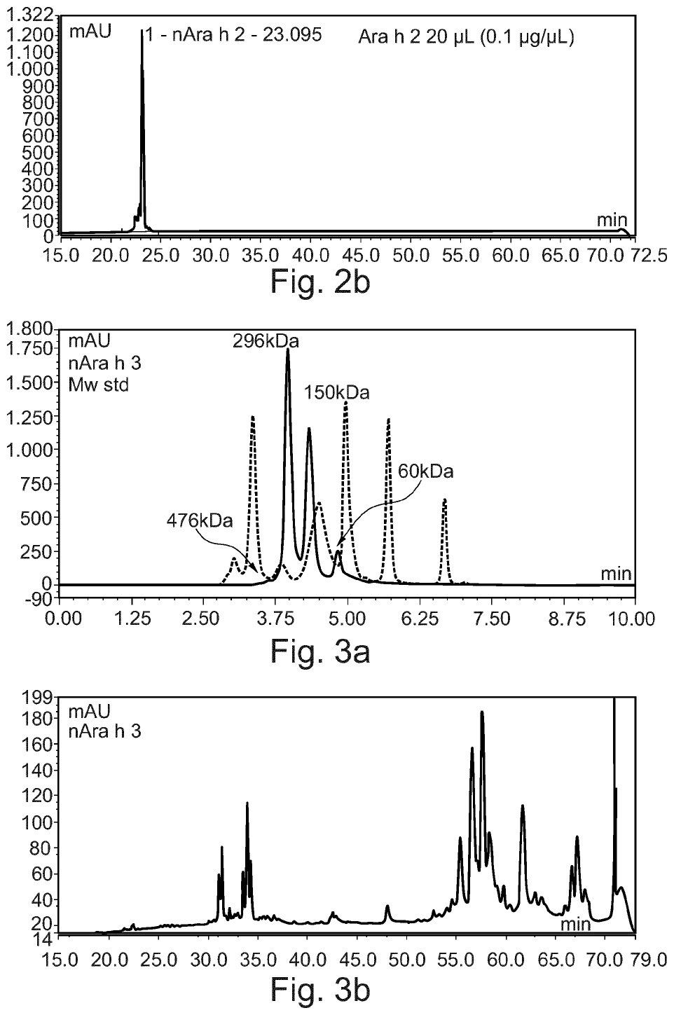

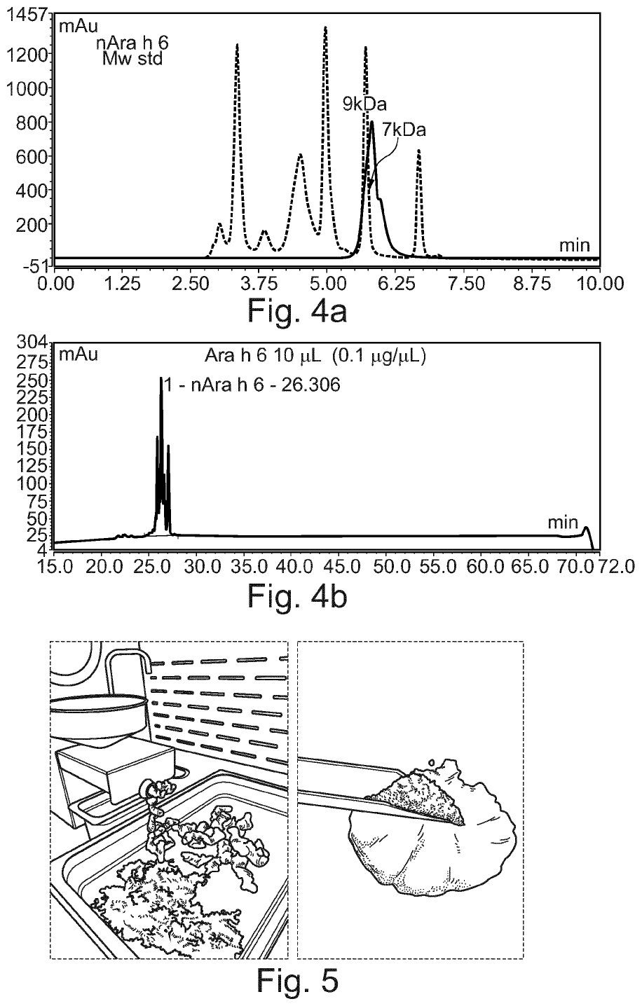

Each of the pure reference standards was evaluated by analytical scale size-exclusion HPLC (SEC X300) and by analytical scale RP-HPLC using the analytical conditions described above. FIGS. 1 to 4 show the resulting chromatograms of the four reference standards from both analytical methods). From the evaluation by SEC analysis, it appears that nAra h 3 elutes with three peaks corresponding to a monomeric (contains one polypeptide chain), trimeric (contains three polypeptide chains) and hexameric conformation (assumably association between two trimeric conformations) of Ara h 3. The hexameric conformation of Ara h 3 has a molecular mass of about 300 kDA when analysed by the present X300 SEC column. Ara h 1 appears as one peak with a molecular mass corresponding to the trimeric conformation of Ara h 1. Ara h 2 and 6 both appear as single peaks with molecular mass corresponding to their monomeric form.

Since the four allergens exist naturally in several isoforms, the RP-HPLC profile of each of the reference standards appears as a clustering of peaks, and the entire cluster may be integrated to correctly determine the peak area assigned to each of the four allergens. In the RP-HPLC chromatograms, the order of elution was: Ara h 2 isoforms, Ara h 6 isoforms, minor fraction of Ara h 3 isoforms, Ara h 1 isoforms and remaining Ara h 3 isoforms.

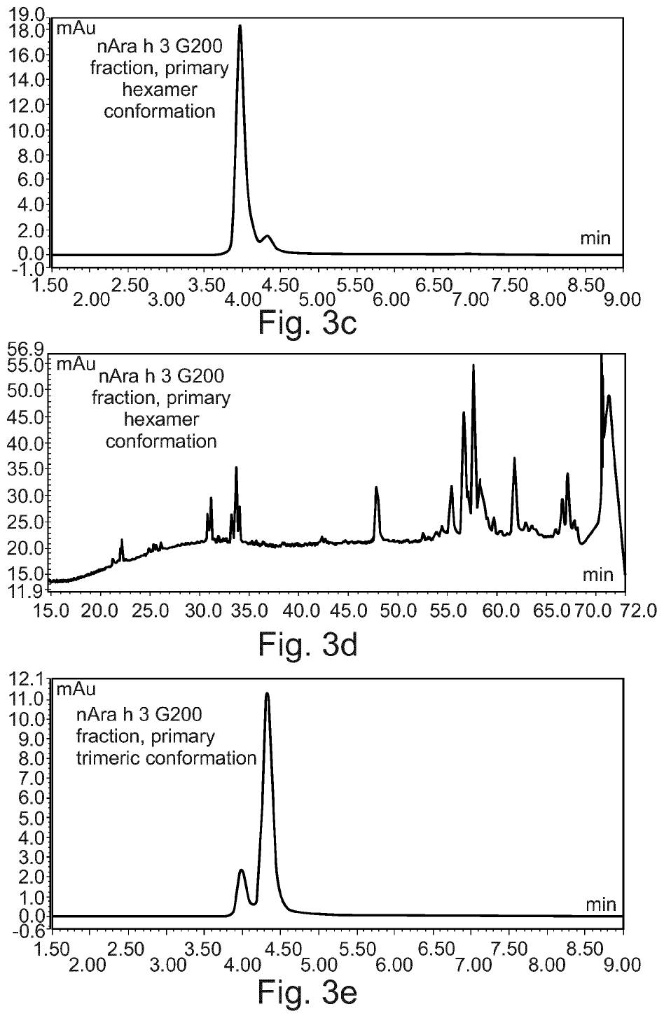

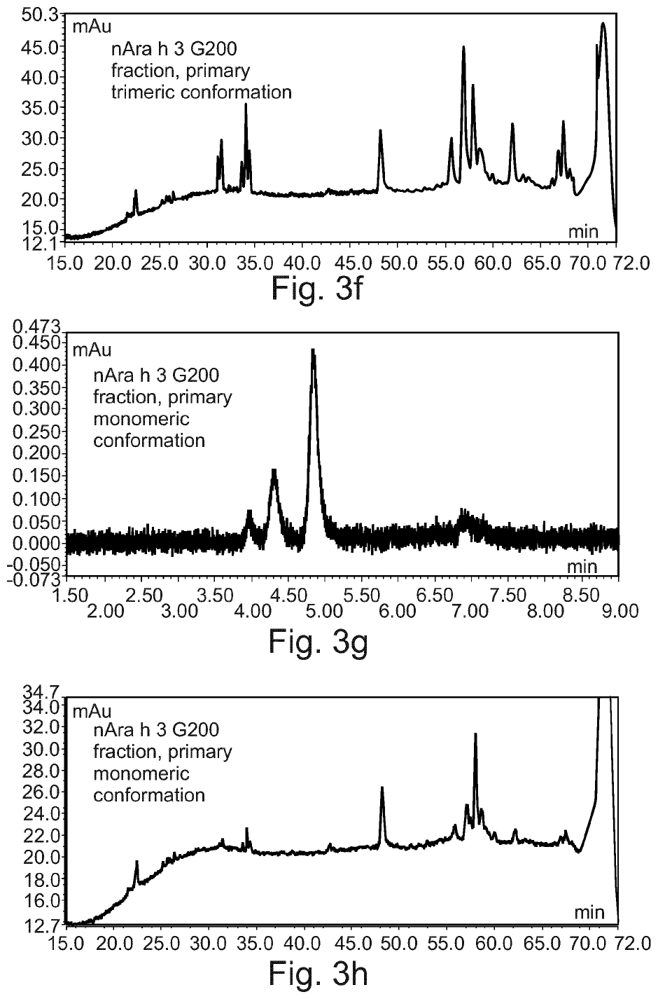

With respect to the allergen nAra h 3, it could be confirmed that the RP-HPLC analysis resulted in the elution of nAra h 3 in its monomeric form despite that the pure standard of nAra h 3 also contains the trimeric and hexameric form. In short: The pure nAra h 3 reference standard was fractionated by preparative SEC using a G200 column. The fractions containing either the hexameric, trimeric and monomeric form were collected and evaluated by analytical scale SEC as well as analytical scale RP-HPLC analysis. FIGS. 3e to 3h show that the injection of either the monomeric, trimeric or hexameric form of nAra h 3 resulted in similar RP-HPLC profiles. Therefore, it has been found that the present RP-HPLC method is suitable for quantification of nAra h 3 by the content of the monomeric form (singe polypeptide form).

The concentration of each of the allergens in the pure fractions was determined by the content of protein measured by amino acid analysis (AAA). Linear calibration curves were obtained by injecting different amounts of the reference standards and determining the peak area for each concentration of each of the four allergens.

The quantitative amount of the four allergens in peanut allergen compositions was determined by injecting a volume (typically between 1 μl and 25 μl) of the peanut allergen compositions and determining the concentration of each of the allergens in mg/ml by use of the linear calibration curve. Linear calibration curves for standardisation could be produced in the following ranges: Ara h 1: 1-10 μg, Ara h 2: 1-3 μg, Ara h 3: 1-12 μg, and Ara h 6: 1-2 μg. The quantitative amount of the allergens in the extracts may further be converted into nmol by use of an average molar mass of each of the allergens (Ara h 1: 68757 g/mol, Ara h 2: 17994 g/mol, Ara h 3: 58600 g/mol and Ara h 6: 14846 g/mol, or the content may be expressed as the ratio normalised to the amount of Ara h 2.

### Example 2

### Defatting/Pulverising of Peanut Kernels

This example relates to the processing of raw peanut kernels to obtain pulverised defatted peanut powder, which can be used as a starting material (peanut source material (PSM)) for the production of a crude allergen extract comprising key peanut allergens.

Raw peanuts (e.g. of the cultivar variant Runner supplied by ACI Seed a.k.a. AgResearch Consultants, Inc) were processed in a mechanical oil press equipped with a screw-style pressing chamber operated at 210° C. to separate crushed defatted peanut material from the peanut oil (FIG. 5). The raw peanuts may be skinned or unskinned, although unskinned is preferred in order to avoid colouring material in the resulting defatted peanut material. The defatted peanut material produced in the first period of processing (about 5 minutes) may be discarded due to heat-induced loss of Ara h 1. The defatted peanut material was collected and allowed to cool before being crushed into flakes. The flakes are further processed by a grinder mill to obtain the final powdered and defatted PSM (FIG. 5). This PSM had an oil content in the range of 8 to 12% by weight.

### Example 3

### Extraction

This example relates to the extraction of the peanut source material (PSM) obtained by the combined defatting and pulverising process outlined in Example 2. The objective is to obtain a peanut allergen extract comprising the four key peanut allergens, Ara h 1, 2, 3 and 6 in their natural conformation.

The most important biochemical characteristics of the four key allergens:

| Parameter | Ara h 1 | Ara h 2 | Ara h 3 | Ara h 6 |
|---|---|---|---|---|
| Molecular mass (g/mol)¹ | 68757 | 17994 | 58600 | 14846 |
| Isoelectric point (PI)² | 6.4 | 5.4 | 5.5-5.7 | 5.5 |
| Heat stability | labile | stable | Stable | Stable |
| Degradation by digestive | sensitive | withstand | Sensitive | withstand |
| enzymes | | | | |

¹The molecular mass is based on the amino acid sequence of the monomeric form of the allergens reported on the homepage http://www.allergen.org/ and as specified in the table below. Where several isoforms are reported, the molecular mass is given as the average molecular mass of the isoforms.
²Theoretical PI calculated from the amino acid seqThe four allergens are represented by the following isoforms:

| | | | Molecular | | |
|---|---|---|---|---|---|
| | | | weight (MW) | | |
| | | | calculations | Number | |
| Allergen | Isoallergen | Uniprot | based on: | of AAA | Mw |
| Ara h 1 | Ara h 1.0101 | P43238 | Sequence | 601 | 68757 |
| | | | 26-626 | | |
| Ara h 2 | Ara h 2.0201 | Q6PSU2-1 | Sequence | 151 | 17994 |
| | | | 22-172 | | |
| | | | Conglutin-7 | | |
| Ara h 3 | Ara h 3.0101 | O82580 | Entire sequence | 507 | 58350 |
| | | | (507 amino | | |
| | | | acids). | | |
| Ara h 3 | Ara h 3.0202 | Q9SQH7 | Sequence | 510 | 58849 |
| Ara h 3 | Ara h 3 | | 21-530 | | 58600 |
| | (average) | | | | |
| Ara h 6 | Ara h 6.0101 | Q647G9 | Chain 22-145 | 124 | 14846 |
| | | | Conglutin | | |

Initially, various extraction conditions were investigated to get insight into the impact of pH, salt concentration, temperature, duration, extraction ratio between PSM and buffer, peanut variant/cultivar, defatting method, powder particle size and the use of roasted vs non-roasted peanuts as peanut source material.

In brief, the extraction was carried out by suspending the PSM in the aqueous extraction buffer under stirring for a period of time, typically between 10 minutes and 2 hours and at a temperature between 5° C. and 25° C., to extract water-soluble allergens from the peanut powder into the aqueous extraction buffer. A crude peanut allergen extract is then obtained by ultracentrifugation (as the liquid supernatant). If the supernatant was turbid or it contained insoluble materials, the supernatant was filtered through a filter, e.g. a filter with a pore size of approximately 1.0 μm as this resulted in a clear aqueous allergen extract. The crude allergen extract may also be subjected to diafiltration to remove buffer salts or other small molecules.

Finally, the extraction of the allergen was carried out by two different buffed aqueous solvents (A and B) to determine the optimal extraction conditions for peanuts of the cultivar variant Runner (mix of genotypes).
A: 100 mM TRIS+180 mM NaCl in purified water, pH adjusted to 8.5 with 2.0 M NaOH. For extraction is used 600 g PMS and 12000 g extraction buffer (1+20 extraction ratio) for one hour at about 10° C. and pH was continuously monitored and adjusted to be at pH 8.5.
B: 50 mM TRIS+50 mM NaCl in purified water, pH adjusted to 7.4 with 2.0 M NaOH. For extraction is used 600 g PSM and 6000 g (=6000 ml) extraction buffer (1+10 extraction ratio) for one hour at room temperature (22° C.) and pH was continuously monitored and adjusted to be at pH 7.4.

The allergen profiles obtained by use of the two extraction buffers were examined by methods described in Example 1 such as reverse-phase HPLC (RP-HPLC) and size-exclusion chromatography (SEC).

Results from Initial Investigation of Extraction Parameters

In general, the four peanut allergens were easily and quickly extracted from the PSM and not much affected by the extraction temperature, duration of extraction and the ratio between the amount of PSM versus extraction buffer volume. The greatest impact was observed at changes in the pH and salt concentration of the extraction buffer.

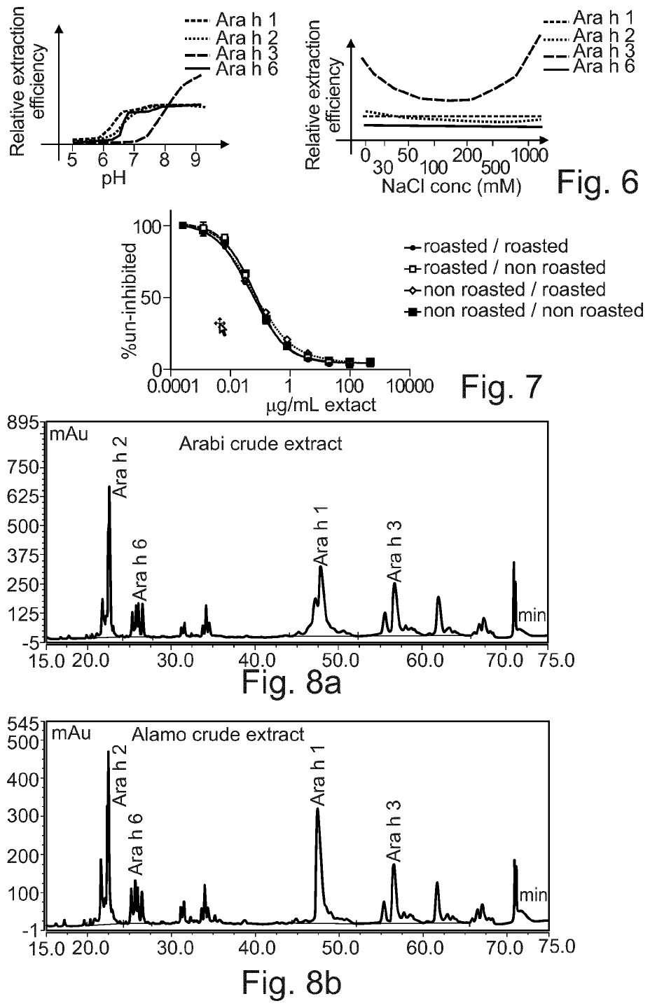

The extraction efficiency of Ara h 3 varied significantly with pH, the maximal extraction of Ara h 3 was reached at pH around 8.5 to 9. At pH below 7, Ara h 3 could hardly be detected in the allergen extracts. The extraction efficiencies of the other peanut allergens Ara h 1, Ara h 2 and Ara h 6 were less pH dependent, although at very low pH values (about pH 2) only Ara h 2 and Ara h 6 could be extracted. Thus, to obtain crude peanut allergen extracts of Ara h 3, the extraction pH should not go below 7. FIG. 6 shows the pH-dependencies of the relative extraction efficiencies between the four peanut allergens from pH 5 to 9 when investigated in aqueous buffers containing 150 mM TRIS and no salt (NaCL). At pH 5, all allergens were not easily extracted as this pH is close to their pI. At pH above 6.5 to 9, the three allergens Ara h 1, 2 and 6 were extracted with same efficiency, whereas Ara h 3 was extracted with increasing efficiency from about pH 7 to 8.5-9.

Concerning the ionic strength, e.g. the salt concentration, the addition of NaCl to the extraction buffers from 30 mM to 1000 mM revealed a biphasic dependency of the extraction efficiency of Ara h 3 with lowest extraction efficiency between 100 mM and 200 mM NaCl. FIG. 6 also shows the relationship between the extraction efficiency of the four allergens and the content of NaCl in the extraction buffer.

The buffer capacity and temperature were found to have minor impact on the extraction efficiencies:

Concerning the impact of buffer capacity, it was found important to use extraction buffers with high buffer capacity in order to maintain the intended pH without the need of adjusting pH during extraction. For example, it was found that TRIS buffer (tris(hydroxymethyl)-aminomethane buffer) with a molar concentration of TRIS of 100 mM or above had the sufficient buffering capacity to maintain the targeted pH value, which is key when extracting allergens with pH-dependent extraction efficiency.

Concerning extraction temperature, there was not found any difference in the allergen extraction efficiency in the investigated temperature from about 5° C. to 25° C. However, the allergen Ara h 1 was found to be thermo-labile and high temperatures should be avoided. For example, the content of Ara h 1 dropped significantly in the crude allergen extract when heated to about 90° C.

Concerning extraction duration, no difference in the yield was detected between 10 minutes and 2 hours duration of extraction.

Concerning the extraction ratio, i.e., the ratio between the amount of PMS and the extraction volume, it was found that the yield of the four peanut allergens was highest at a ratio of 1:30 (e.g 100 g PMS dispersed in 30 kg (=30 liter)) of extraction buffer, and lower at ratios of 1:20 and 1:10. Lower extraction ratios may be used, but turbidity or precipitation of proteins may appear.

The selection of the peanut variant and the defatting method/pulverising method would have some impact on the allergen profiles of the crude allergen extracts:

For example, it was found that the HPLC allergen profiles of crude allergen extracts made from PMS obtained from peanuts of varying degree of roasting (none, light and dark roasted peanut flour) at pH 8.5 showed that roasting affects the allergen profile. The RP-HPLC peaks of Ara h 2, Ara h 3 and Ara h 6 in the crude extracts of roasted peanuts appeared flatter, although the thermo-stabile allergens Ara h 2 and Ara h 6 were not as affected as the thermo-labile allergen. Notably, the peak of Ara h 1 was completely absent in the crude allergen extracts of dark roasted peanut and considerably reduced in the crude allergen extracts of lightly roasted peanuts. This phenomenon was also observed by analysis with SDS-PAGE. It is hypothesised that the difference in the profiles between raw and roasted peanuts is caused by Maillard reactions and glycosylations of peanut proteins during roasting.

By human IgE-inhibition ELISA, it was however confirmed that extracts obtained from roasted peanuts or from raw peanuts both could completely inhibit each other's signal. Therefore, it was concluded that allergen extracts obtained from extraction of roasted peanuts did not contain any unique IgE-epitopes which are not present in an allergen extract of raw peanuts. Peanut extracts from roasted and non-roasted (or raw source material) showed similar and full inhibition, when tested by use of a panel of serum positive for sIGE to peanut extract (n=12; United States (4), United Kingdom (1), Germany (4), Switzerland (1), Sweden (1), Denmark (1)). The IgE-inhibition ELISA is based in the competition between free antigen (inhibitor, whole peanut extract, raw or roasted) and the corresponding antigen (whole peanut extract, raw or roasted) absorbed to the plate for binding to serum IgE: 96-well ELISA plates were coated with either 1 μg of raw peanut extract or 1 μg roasted peanut extract in PBS (phosphate buffered saline with pH 7.4) overnight at 4° C. After washing with PBS/Tween-20, plates were blocked with PBS/1% Casein for 2 hours at room temperature. Plates were washed with PBS/Tween-20 and incubated at 37° C. for 1 hour with a dilution of the serum sample in the presence of increasing concentrations of competitor. Competitors (free antigen) consisted of increasing concentrations of whole peanut extract, raw or roasted. The concentrations used for the free allergen competitor were in the range of 0.0005-500 μg. After washing, the plates were incubated with an anti-human IgE antibody directly conjugated HRP for 1 hour at room temperature. Colour was developed using “TMB one” (3, 3′, 5, 5′-tetramethylbenzidine is a chromogenic substrate for Horseradish Peroxidase), and the OD of each well was measured at 450 nm.

FIG. 7 shows a representative plot of IgE inhibition assay performed on a serum from a donor living in the USA. This serum contained sIgE to Ara extract (53 kU/L), Ara h 1 (11 kU/L), Ara h 2 (28 kU/L), Ara h 3 (4 kU/L) and Ara h 6 (15 kU/L). Here, it is shown that there is no difference in the capability of achieving full inhibition with either non-roasted or roasted peanut extract. This indicates that no new IgE epitopes are generated due to roasting of peanuts. Irrespective of which extract (non-roasted or roasted) that was used for coating the ELISA plates, the individual sera from the entire panel could be inhibited to the similar capacity with roasted or non-roasted peanut extracts.

The table below shows the peak areas of the four key peanut allergens in HPLC chromatograms obtained from analysis of crude allergen extracts made from raw peanuts, lightly roasted peanuts and dark roasted peanuts, respectively. All peanuts were of the cultivar “Spanish” and the roasting was performed in-house at 350 F (177° C.) for 20 minutes to obtain “lightly roasted” peanuts and for 50 minutes to obtain dark-roasted peanuts.

HPLC peak areas (mAU*min)

| Peanut Sample | Ara h 1 | Ara h 2 | Ara h 3 | Ara h 6 |
|---|---|---|---|---|
| Non-roasted | 127 | 130 | 306 | 52 |
| Light-roasted | 7 | 42 | 77 | 16 |
| Dark-roasted | 0.6 | 5 | 8 | 0.2 |

Further, the HPLC allergen profiles of crude allergen extracts from extraction of commercially available lightly roasted peanut flour (Golden Peanut Company) in TRIS buffer at pH 8.5 showed that the chromatographic peak of Ara h 1 was missing (Data not shown).

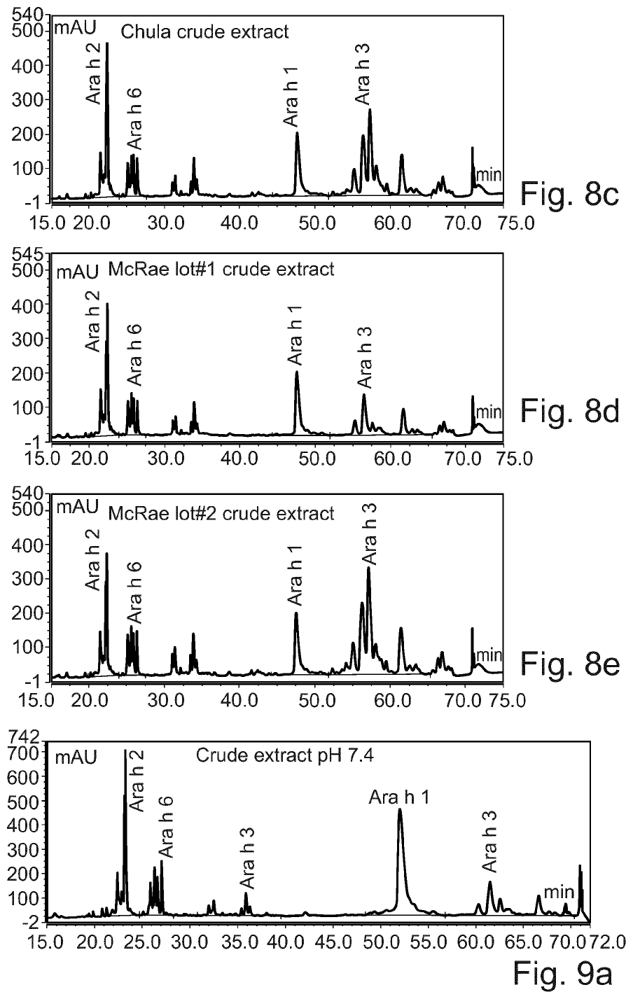

Cultivated peanut (Arachis hypogaea) comes in many botanical varieties, but there are four basic types: Runner, Virginia, Spanish and Valencia. Each of the peanut types is distinctive in size, flavor, and nutritional composition, and the cultivar variety Runner is the most widespread peanut variant. Each cultivar variety also comes in different genotypes. FIG. 8 shows the RP-HPLC profiles of crude allergen extracts made from different Runner genotypes (#1041, #3310 and #212C) and indicates slightly different RP-HPLC profiles, in particularly in the part of the chromatogram where Ara h 3 appears. Notably, the genotypes #3310 and #212C do not have the peak seen with the genotype 1041. Notably, it was found that this extra peak in the 1041 genotype was recognised by human IgE when analysed by western blot technology. Therefore, it may be recommended to produce allergen extracts obtained from the 1041 genotype, eventually in mixture with other Runner genotypes or cultivar variants.

The following shows the amount of each of the four key allergens in allergen extracts obtained from raw peanut (Runner, mix of genotypes). The content is reported in mg/ml, nmol/ml or as the molar ratio versus Ara h 2.

| Extraction | Amount in mg/ml, (nmol/ml), molar ratio vs Ara h 2 | | | |
|---|---|---|---|---|
| buffer | Ara h 1 | Ara h 2 | Ara h 3 | Ara h 6 |
| A (pH 8.5) | 1.7 | 1.4 | 4.1 | 0.8 |
| | (25) | (78) | (70) | (56) |
| | 0.3 | 1.0 | 0.9 | 0.7 |
| B (pH 7.4) | 3.3 | 1.4 | 2.3 | 1.1 |
| | (47) | (78) | (40) | (75) |
| | 0.6 | 1.0 | 0.5 | 1.0 |

FIG. 9 shows the HPLC profile (long run method) of the crude peanut extracts made with the extraction buffers mentioned above: Buffer B (pH 7.4) versus Buffer A (pH 8.5). The data indicates that Ara h 1 is extracted to a lower degree at higher pH (8.5) than at pH 7.3, while this is opposite for Ara h 3. Therefore, it is challenging to generate compositions comprising balanced concentrations of the four key allergens by simple extraction of peanuts.

### Example 4

### Purification of an Allergen Extract

This example relates to the purification of crude peanut allergen extracts, such as crude allergen extracts obtained in Example 3.

The allergen extract obtained in Example 3 was loaded onto an anion exchange chromatographic column to adsorb the four key allergens to the anion exchange material, optionally together with other peanut allergens of interest. The liquid that passes directly through the column by this operation was collected as an individual fraction (named the “flow-through fraction” (FT)). This fraction contains compounds of the allergen extract, which are poorly adsorbed to the anion exchange material, e.g., proteinic or non-proteinic compounds without or with poor negative charging. Some minor peanut allergens are present in the flow-through fraction like Ara h 8 and Ara 9.

With the aim to obtain individual fractions enriched with one of the key allergens (i.e. a fraction enriched with Ara h 1, a fraction enriched with Ara h 2, a fraction enriched with Ara h 3 and a fraction enriched with Ara h 6), individual liquid fractions were collected upon elution with gradually increasing concentrations of salt (sodium chloride (NaCl)) in the elution buffer. Alternatively, the salt gradient may be accomplished by stepwise elution with individual volumes of aqueous elution solvents having different salt concentrations. The anion exchange material was a strong anion exchange resin, HiTrap Q HP from Cytiva™. It was packed with Q Sepharose High Performance strong quaternary ammonium anion exchange resin, has a small (34 μm) bead size and delivers high-performance, high-resolution purifications. The resin was packed in a column (diameter 100 mm) and equilibrated with buffer (25 mM TRIS, HCL pH=7.4). The column was operated with a flow rate of about 6CV/hr and the collected fractions were evaluated for the allergen content by use of the HPLC procedure described in Example 4. Different fractions may then be mixed to give a desired allergen composition with respect to the intended amounts of each of the key peanut allergens, such as the relative molar ratio to Ara h 2.

Different salt gradients were examined in that the two allergens Ara h 2 and Ara h 6 were difficult to separate to obtain individual enriched fractions. It was found that the lowering of the pH from pH 8.5 to pH about 7.5 provided a better separation of the two allergens and made it possible to obtain individual fractions enriched with Ara h 2 or with Ara h 6.

By example, the four key peanut allergens could be collected in four individual fractions, wherein each fraction comprises a high enrichment for one of the four key peanut allergens. This anion exchange chromatographic procedure comprises the following steps:
A peanut allergen extract obtained by extraction with buffered aqueous solution having pH 7.4 as described in Example 2 (e.g. 50 mM TRIS buffer, pH 7.4+50 mM NaCl) was loaded onto the influx end of the anion exchange column using a flow rate of 6CV/hr.
The flow-through liquid was collected at the efflux end of the anion exchange column and is named the flow through fraction (FT);
Stepwise elution with increasing salt concentrations was carried out four times in total in that each step comprised: i) elution with an aqueous elution solvent consisting of 25 mM TRIS, HCL pH=7.4 and a given pre-determined amount of salt (NaCl) at a flow rate of 6CV/hr and ii) collection of the efflux liquid in individual fractions of the loaded allergen extract, wherein
   the first elution liquid comprises a salt content of 125 mM NaCl and the resulting fraction (A) was enriched with Ara h 6;
   the second elution liquid comprises a salt content of 200 mM NaCl and the resulting fraction (B) was enriched with Ara h 2;
   the third elution liquid comprises a salt content of 375 mM NaCl and the resulting fraction (C) was enriched with Ara h 1; and
   the fourth elution liquid comprises a salt content of 700 mM NaCl and the resulting fraction (D) was enriched with Ara h 3.

Any further elution of the anion exchange column with a higher salt concentration of up to 1000 mM NaCl did not result in fractions containing the four key allergens Ara h 1, 2, 3 and 6 when investigated by RP-HPLC.

The allergen profiles of the collected fractions were examined by methods described in Example 1 such as reverse-phase HPLC using the short-run method and size-exclusion chromatography (SEC).

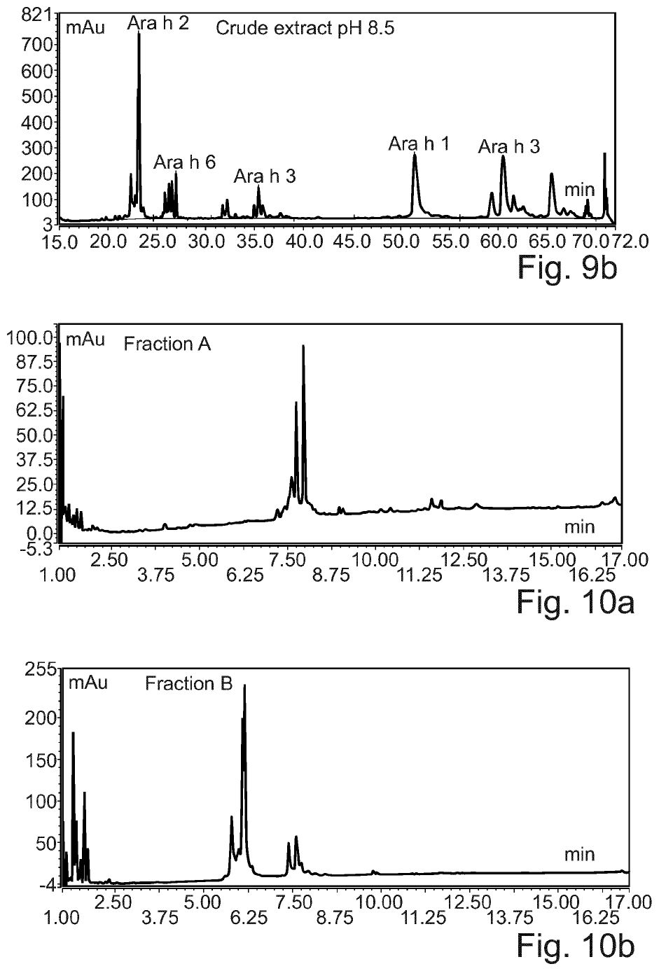

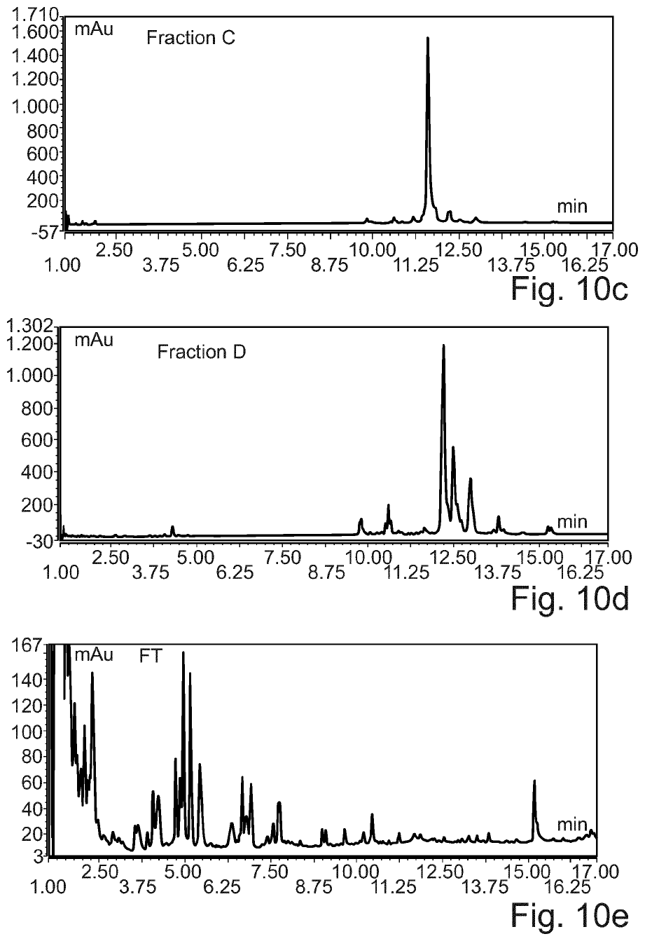

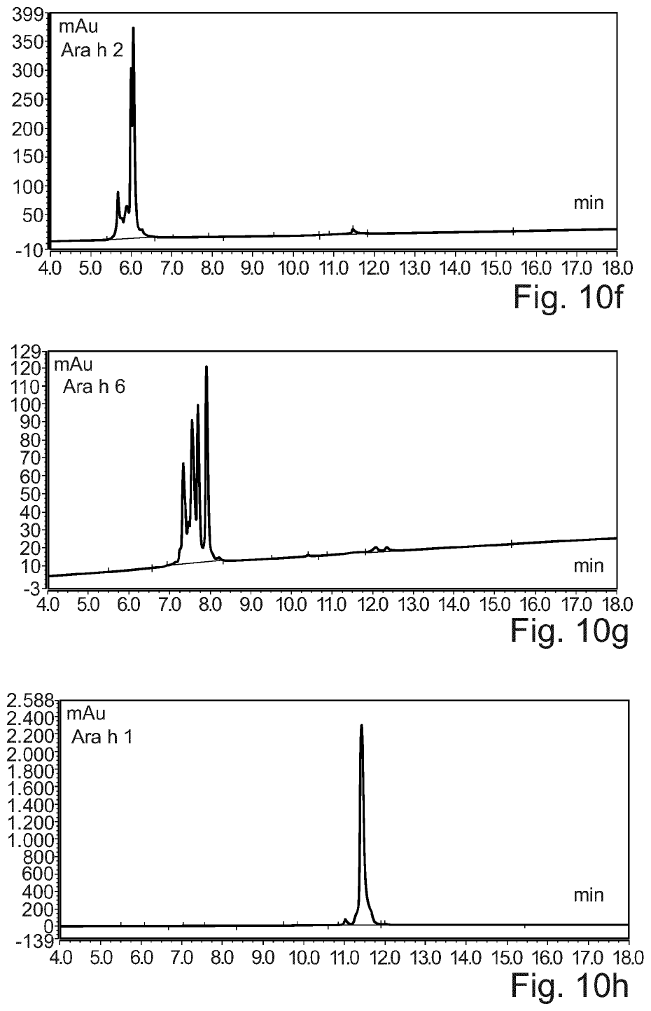

The RP-HPLC chromatograms (short run method) of fractions A, B, C and D confirm that the anion exchange chromatography process as outlined above was able to produce individual fractions with high purity for either of the peanut allergens Ara h 1, 2, 3 and 6 (FIGS. 10a-d) and that the flow through (FT) fraction was enriched for other allergens than Ara h 1, 2, 3, and 6 (FIG. 10e). Pure standards of nAra h 1, 2, 3, and 6 are shown in the FIGS. 10f to 10i.

### Example 5

### Mixing of Fractions to Final Drug Substance

This example relates to the mixing of the fractions A, B, C and D, and optionally the fraction FT, obtained in Example 4 to produce peanut allergen compositions comprising targeted levels of one or more specific peanut allergen(s), which may be used as drug substance (DS) for treating peanut allergy, such as by allergen-specific immunotherapy. The mixed fractions may be used as is, or may further be processed, such as to remove undesirable constituents (e.g., buffers, salts, small-size molecules), to up-concentrate the final liquid peanut allergen composition or to change the pH values. The liquid peanut allergen composition may also be subject to lyophilisation or droplet freezing.

Following determination of the concentration of each of the four key allergens by RP-HPLC in all the fractions collected in Example 4, peanut allergen compositions with targeted amounts of peanut allergens were produced by mixing appropriate aliquots of one or more of the fractions.

By example, the entire volume of the flow-through liquid collected in Example 4 and aliquots of each of the four enriched fractions A, B, C and D was mixed to produce peanut allergen compositions with targeted molar ratios between the four key allergens being close to 1:1:1:1 (balanced amounts). By example, the enriched fractions may be mixed to obtain purified peanut allergen extracts with molar concentrations of each of the three key allergens Ara h 1, 2, and 6 relatively to the molar concentration of Ara h 2 being in the range of a relative molar ratio of 0.5 to 1.5 or more narrower, such as a relative molar ratio 0.4 to 1.4. Such peanut allergen compositions may also contain other peanut allergens that have co-eluted with the four key peanut allergens. For example, peanut allergen Ara h 7 is present in the fractions containing Ara h 2 or 6, and a number of the minor peanut allergens (e.g. Ara h 8, 9, 12 and 13) are present in the flow-through liquid. Compositions with other molar ratios can be produced and it can be decided to leave out one or more the fractions A, B, C and D to produce peanut allergen compositions with low levels of one or more of the four key allergens or minor allergens. Thus, by using the processes described in the examples 3 to 5, peanut compositions comprising controlled and pre-selected amounts of Ara h 1, 2, 3, and 6 can be provided even to the extent that the peanut composition does not contain one, two or three of the peanut allergens Ara h 1, 2, 3, and 6, which simply can be made by excluding one or more of the enriched fractions.

Following mixing, the liquid peanut allergen composition was then up concentrated by ultrafiltration step at 22° C. using membrane cut-off at approximately 5 kDa MW to remove smaller molecules, e.g. buffer salts (TRIS buffer) and to reduce the volume about 25 times. Further low molecular mass molecules are removed by diafiltration until the conductivity is about 650 mS/cm. The peanut protein content in the final purified extract was in the range of 16 to 21 mg/ml when determined by amino acid analysis (AAA).

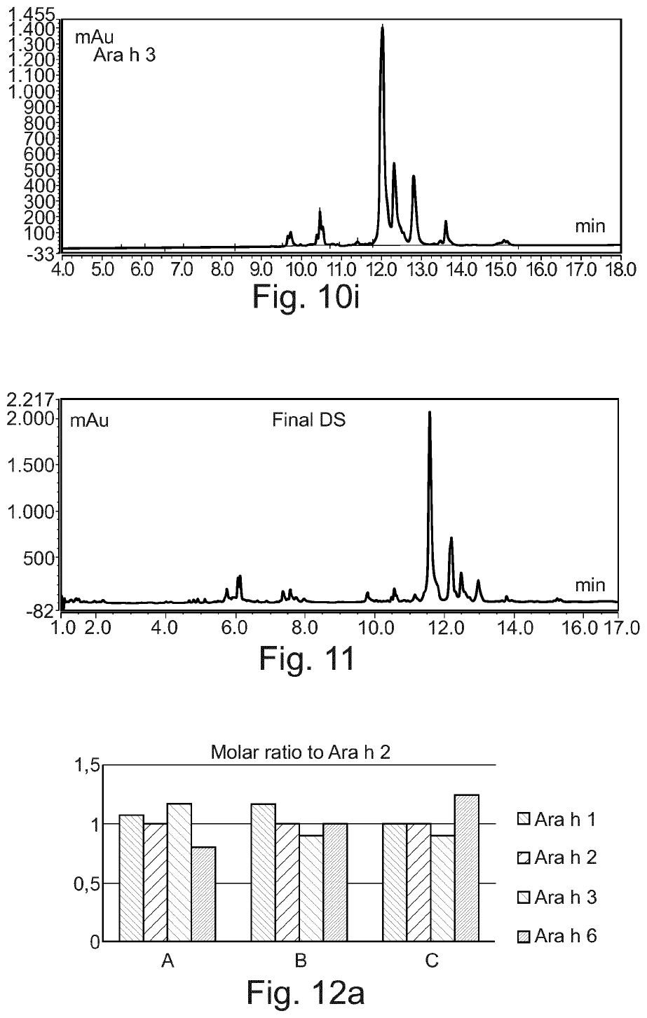

A typical RP-HPLC chromatogram (short run method) of a purified allergen extract is shown in FIG. 11. The purified allergen extract was made by mixing the FT fraction and appropriate volumes of fractions A, B, C and D to produce an extract with balanced molar amounts of each of the allergens Ara h 1, 2, 3, and 6 and with content of minor allergens present in the FT fractions. By example, the FT fractions may be left out or be added in a minor volume.

Below is shown the results of the amounts of the four allergens in a typical composition obtained by the methodology described in example 5. The results obtained by RP-HPLC and LC-MS/MS are compared and reveal good consistency between the two methods.

| | % allergen by weight of | | nmol allergen/mg | |
|---|---|---|---|---|
| | peanut protein | | peanut protein | |
| Allergen | RP-HPLC | LC-MS/MS | RP-HPLC | LC-MS/MS |
| Ara h 1 | 39.2 | 36.2 | 5.7 | 5.3 |
| Ara h 2 | 7.9 | 9.8 | 4.4 | 5.4 |
| Ara h 3 | 33.3 | 38.4 | 5.7 | 6.6 |
| Ara h 6 | 6.7 | 7.9 | 4.5 | 5.4 |
| sum | 87.1 | 92.3 | | |

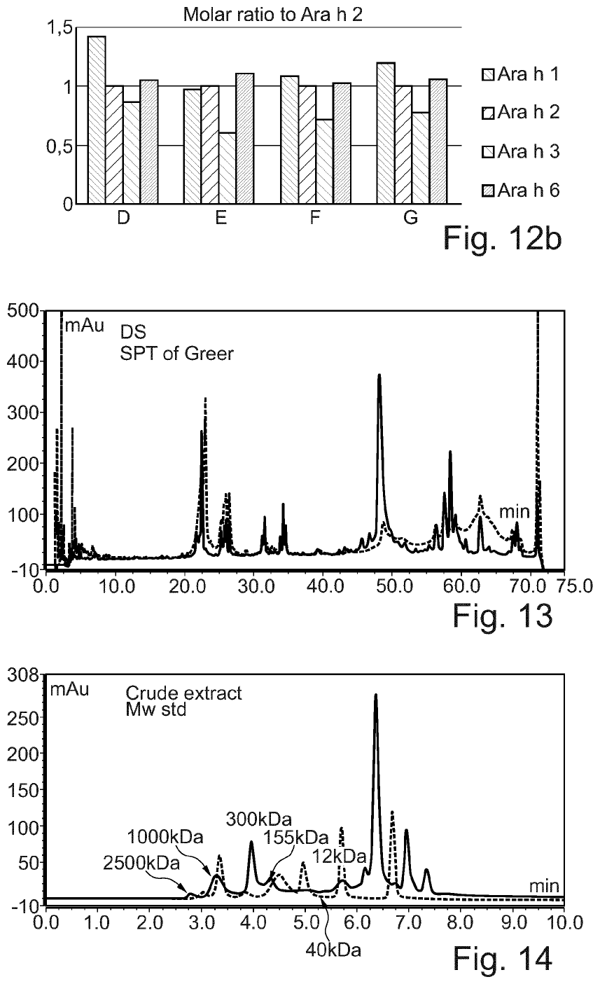

Three batches of purified allergen extract (Batches A, B and C) were produced with a targeted molar ratio between the molar amount of each of the allergens Ara h 1, Ara h 3 and Ara h 6 versus the molar amount of the allergen Ara h 2 close to 1:1:1. The molar concentrations of the four key allergens and the molar ratio to Ara h 2 were determined in the three batches A, B and C as well as in four batches of a comparator allergen extract (commercially available peanut allergen extract sold for doing skin prick testing (SPT product of Greer)). The molar concentrations were determined by LC-MS/MS combined with isotope labelled AQUA peptides. Spike-in of pure reference standards of nAra h 1, nAra h 2, nAra h 3 and nAra h 6 were used to verify the allergen amounts and the quantification method. FIG. 12a shows the relative molar amounts in the three batches (A, B and C) and reveal high consistency to the targeted molar concentrations and low variation between batches. FIG. 12b illustrates the variation in the relative ratios within the same batch as well as between batches of four batches of the Greer SPT product (D-G) when analysed in a similar set-up using heavy isotope labelled AQUA peptides combined with LC-MS/MS analysis.

The HPLC allergen profiles of Batch A (produced according to the examples 2 to 5) were compared to a SPT product of Greer. FIG. 13 shows the overlay of the RP-HPLC profile of Batch A and of a batch of the SPT Greer allergen extract (Batch D). Notably, the RP-HPLC profile of Batch A comprises peaks easily detectable as originating from nAra h 1, 2, 3, or 6, whereas the Greer SPT product comprises flattened peaks in the part of the chromatogram where nAra h 1 and nAra h 3 should appear. Upon injecting the Greer SPT peanut allergen extract a number of times, the column got clogged. Therefore, the allergen levels of the SPT Greer peanut allergen extract cannot be controlled and quantified by use of RP-HPLC method, whereas the RP-HPLC method is eligible for being used as an accurate method for controlling the levels of each of the allergens nAra h 1, 2, 3, and 6 in the purified allergen extracts of Example 4. Furthermore, the lack of nAra h 1 as a single peak in the RP-HPLC profile of the Greer SPT, may indicate that nAra h 1 is not present in the Greer extract in a conformation that can be analysed by RP-HPLC. It appears that the LC-MS/MS quantification method, which uses digestion of the allergens into smaller peptides before quantification would not return the correct amount of nAra h 1 in terms of monomeric amount, but also would measure oligomerised nAra h 1. The same is observed for nAra h 3, wherein the LC-MS/MS method will return levels of all peptides deriving from a Ara h 3 sequence, including those present in a oligomerised nAra h 3. The RP-HPLC method which determines the nAra h 3 single polypeptides seems to be more accurate for determining the content of nAra h 3 deriving from a monomeric, trimeric or hexameric nAra h 3, as oligomeric forms will clog the HPLC column or fails to elute with a sharp identifiable chromatographic peak.

Further by use of size exclusion chromatography (SEC), the allergen profiles of the purified allergen extracts obtained in Example 5 were compared to the crude allergen extract of Example 3 that was obtained before anion exchange chromatography and mixing.

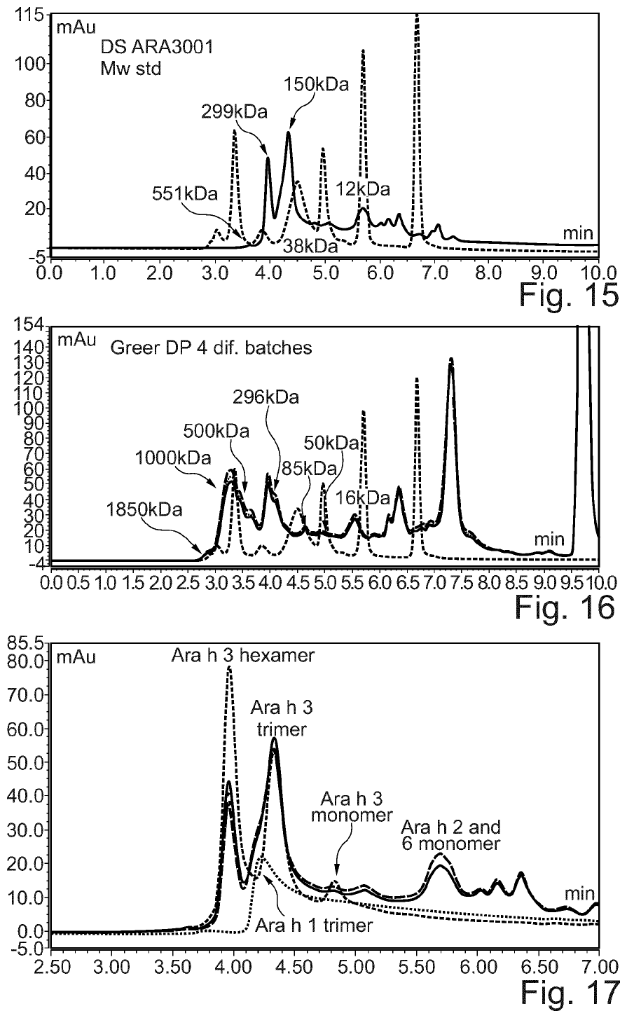

Notably, the crude allergen extract of Example 3 comprises high-molecular mass molecules having molecular mass that are significantly higher than 400 to 500 kDa) than observed for each of the pure standards of nAra h 1, 2, 3, and 6 (FIG. 14) while the high-molecular mass molecules are substantially absent in the purified allergen extract of Example 5 (FIG. 15). The SEC analysis of the SPT product of Greers showed significant levels of high-molecular mass molecules (FIG. 16). FIG. 17 shows the SEC analysis of three batches of purified allergen extracts obtained in Example 5 (using the process steps of example 2 throughout example 5) and demonstrates low batch-to-batch variation in the levels of the different conformational forms of Ara h 3 and Ara h 1. It was further found that the ratio between nAra h 3 trimer and nAra h 3 hexamer is higher in the purified allergen extracts of Example 5 compared to the crude allergen extracts of Example 3. It seems that the anion exchange fractioning changes the nAra h 3 conformations into more of the trimeric form (data not shown).

The origin of the high molecular mass molecules was further investigated after isolation of the high molecular mass molecules by size exclusion chromatography. By RP-HPLC analysis and LC-MS/MS AQUA peptide analysis, it was found that the high mass molecules comprise nAra h 1 and nAra h 3 polypeptides. This is further verified by native gel electrophoresis as shown in Example 6. Therefore, it is speculated that the high mass molecules are aggregates of nAra h 1 and/or nAra h 3 polypeptides.

By shotgun proteomics, it was further confirmed that all the peanut allergens listed in WHO/IUIS Allergen Nomenclature were present in extracts comprising the five fractions, A to D as well as the flow-through fraction (FT).

### Example 6

### Solid Dosage Forms

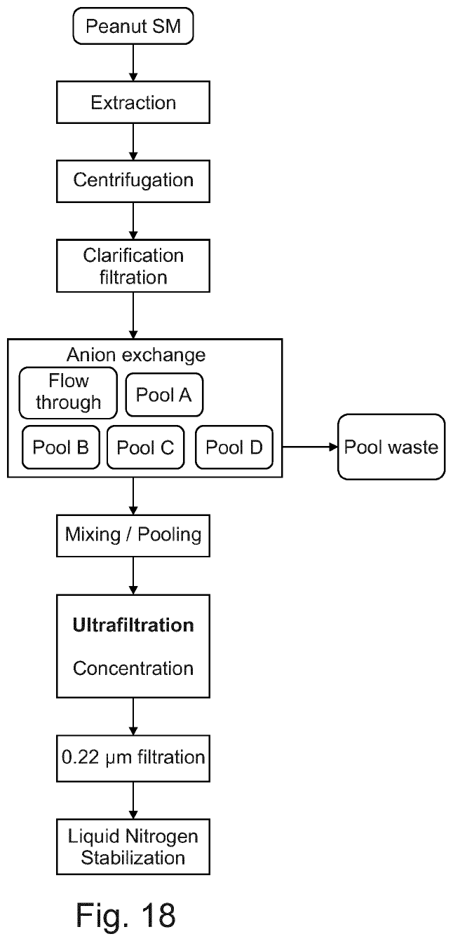

The resulting peanut allergen composition may further be formulated into a pharmaceutical dosage form, either without further processing or following lyophilisation or treatment by liquid nitrogen to produce free flowing frozen droplets. FIG. 18 shows the flow chart of a typical manufacturing process starting from the peanut source material, through the steps of extraction, fractionation and mixing of fractions. Following mixing, the composition may be subjected to ultracentrifugation and filtration. Finally, the liquid composition may be treated with liquid nitrogen to store the composition as frozen droplets before being further formulated into a pharmaceutical formulation, e.g., dosage form.

By example, the resulting peanut allergen composition may be lyophilised and then mixed with pharmaceutical inactive ingredients to produce a sublingual dosage form such as a film, compressed or non-compressed tablet or a lyophilised unit dose form (e.g in the form of a tablet).

Alternatively, the resulting liquid peanut allergen composition may be mixed with a gelatin (optionally mixes of different size gelatins of different origins) and mannitol and then lyophilised to obtain a fast-dispersing solid dosage form suitable for sublingual administration. Advantageously, it has been possible to provide solid dosage forms comprising a total peanut protein content in the range from about 0.1 microgram to about 5000 microgram with low variation in the content of Ara h 2 by weight relatively to the total peanut protein content and wherein each of the allergens Ara h 1, Ara h 6, and Ara h 3 is present in molar levels relatively to the molar levels of Ara h 2 in the range of 0.5 to 1.5 or more narrow. As explained above, the RP-HPLC method described in Example 1 is a suitable method for controlling the molar concentrations and ratios. Alternatively, the concentrations may be controlled by antibody-based assays (e.g., ELISA) as described in Example 8.

The four key allergens were found to be stable in the solid dosage forms for at least 12 months at room temperature about 25° C., which allows for storing the solid pharmaceutical compositions disclosed herein at room temperature.

### Example 7

### Controlling Peanut Proteins by Native Gel Electrophoresis (Native PAGE)

Native gel electrophoresis can be used to compare and control protein patterns of crude extracts (as produced according to Example 3), purified extracts (as produced according to Example 4 and 5) of final lyophilised solid dosage form containing gelatin and mannose (produced according to Example 6) to get information about content of high-molecular size proteinic structures.

Native PAGE was run with Bis-Tris 4-16% gel (Invitrogen®), using reagents from Invitrogen®: NativePAGE Sample Buffer (SB), NativePAGE 20× Running Buffer, NativePAGE 20× Cathode Buffer Additive, Native Mark Protein std and unstained Protein Standard. The electrophoresis was carried out without heating of samples. Protein bands in the size range above 700 kDa were excised and treated with the enzyme trypsin or chymotrypsin for mass spectrometry analysis.

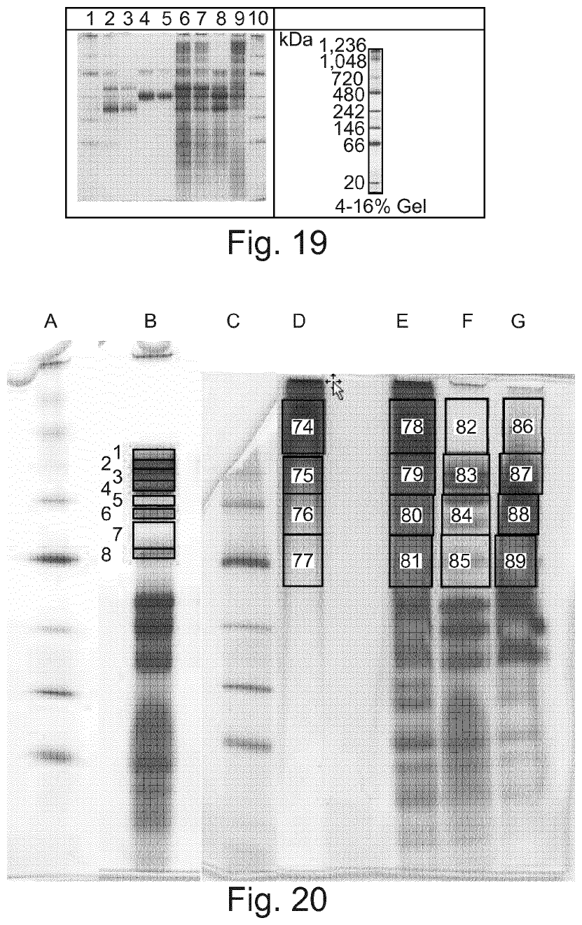

### Results are Provided in the FIGS. 19 and 20:

FIG. 19 shows the native gels of purified standards of nAra h 3 (lanes 2(4 ug) and 3(2 ug)) and nAra h 1(lanes 4 (4 ug) and 5 (2 ug)) and crude extract (filtered) (lanes 6 (30 ug) and 7 (15 ug)) purified crude extract (lane 8 (15 ug)) and comparator peanut allergen extract (Greer) extract (lane 9 (15 ug). Molecular size indicators (lanes 1 and 10). Notably, the purified allergen extract did not contain proteinic structures with sizes above 700 kDa as the longest eluted band appears at a size about 480 kDa, and the ingredients of the placebo solid dosage form (gelatin and mannose) did not induce detectable such high-molecular size proteinic structures.

FIG. 20 shows the protein bands eluting on native electrophoresis gel from the following samples: A) Standard size marker; B) crude extract (filtered); C) standard size marker; D) placebo solid dosage form; E) crude extract (filtered) added to placebo; F) crude extract (filtered); and G) solid dosage form formulated with purified extract. Protein bands in the size range above 700 kDa of lane B were investigated by mass spectrometry analysis. It was found that bands 1 to 6 consisted of Ara h 1 protein structures and bands 7 and 8 consisted of mixed Ara h 1 and Ara h 3 structures. Similar bands in the size range above 700 kDa of lanes D, E, F and G were also investigated by MS. For sample D, there was not detected protein structures corresponding to those of bands 1 to 8, while these could be detected in the samples E and F, and sporadically in sample G. Thus, the process of formulating the purified extract into a solid dosage form did not generate notably amounts of high-molecular size proteinic structures.

### Example 8

### Determining Concentrations of Allergens by Quantitative Immune Assay in Extracts and Final Dosage Form.

The concentration of nAra h 1, nAra h 2, nAra h 3 and nAra h 6 in compositions disclosed herein may be determined/controlled by quantitative immune assay using monoclonal antibodies able to specifically bind either of the peanut allergens nAra h 1, nAra h 2, nAra h 3 or nAra h 6.

Monoclonal antibodies may be purchased from vendors, for example from Indoor Biotechnologies® or they can be generated in-house after immunising mice or rabbits with either purified nAra h 1, nAra h 2, nAra h 3 or nAra h 6 and selecting clones for monoclonal recombinant expression of antibodies (mAbs). Preferred antibodies are murine IgG2a antibodies, which advantageously can be purified by Protein A binding. Ideally, the mAbs should bind the allergens with high affinity, bind non-overlapping epitopes, bind epitopes conserved among all isoforms and with no cross-reactivity between the different allergens. The mAbs may be expressed in HEK cells and purified by protein A affinity chromatography. The purity and size may be controlled by SDS-PAGE and specificity and binding affinity to selected peanut allergens may be controlled by ELISA.

Conventional or automated ELISA-based immunoassays were set-up using specific mAbs against nAra h 1, nAra h 2, nAra h 3 and nAra h 6. Standard curves were prepared using the purified standards of each of the four allergens (as described in Example 1).

nAra h 3 appears with several protein bands at SDS-PAGE gel and it was important to select mAbs specific for Ara h 3, which can bind a majority or all the various protein bands appearing in the SDS-PAGE gel for nAra h 3. This is advantageous, since mAbs only binding one or a few of the protein bands will underestimate the concentration of nAra h 3 in extracts or final dosage forms. Furthermore, a suitable mAbs might be able to bind the nAra h 3 epitopes independent on nAra h 3 being in the monomeric or its oligomeric forms. Thus, a monoclonal anti-Ara h 3 antibody might be able to bind i) Ara h 3 in its monomeric conformation as 1:1 complexes, ii) Ara h 3 in its trimeric conformation as 3:1 complexes (three anti-Ara h 3 antibodies bind one Ara h 3 trimer) and iii) Ara h 3 in its hexameric conformation as 6:1 complexes (six anti-ara h 3 antibodies bind one hexameric Ara h 3).

By example, it was found that the concentration of nAra h 3 in a purified extract produced according to Example 5 (mixed enriched fractions of nAra h 1, 2, 3, and 6) could be assessed by automated ELISA-based immunoassay (Gyrolab® platform) to about 242 ug nAra h 3/mg peanut protein when the ELISA was performed with a specific monoclonal anti-nAra h 3 antibody, which only binds a few of the protein bands appearing on SDS-PAGE for nAra h 3. In contrast, the concentration was about 418 ug nAra h 3/mg peanut protein when the ELISA was performed with a specific monoclonal anti-nAra h 3 antibody, which binds all bands of nAra h 3 on SDS-PAGE gel.

### Example 9

### Updosing Schedule for Immunotherapy

This example relates to the investigation of an updosing regimen (UDR) for use in allergen-specific immunotherapy in patients diagnosed with peanut allergy. The trial medication will be a peanut allergen composition comprising targeted amounts of selected peanut allergens, such as a composition disclosed herein, i.e. composition comprising a high quantity of each of the four key allergens per mg peanut protein. The peanut allergen composition may be produced according to the examples 2, 3, 4, 5 and 6 described herein. During and after the course of immunotherapy, the peanut allergic patients will be able to mitigate allergic reactions that may occur with accidental exposure to peanut. By desensitising patients to peanut allergens, the tolerance threshold for peanut is increased, and consequently protects against allergic reactions upon accidental peanut exposure.

The aim is to provide a safe updosing regimen with few updosing steps, while it is still possible to reach a high tolerable maintenance dose for continued immunotherapy.

By conducting the trial, it will be confirmed that the updosing regimen is safe and can be started at a relative high dose of peanut protein, such as in the range of 10 to 150 microgram peanut protein and that the updosing can be done in a few steps, such as at most five steps to reach a considerable high maximum dose of 4000 microgram of peanut protein.

### Objectives

The primary goal is to evaluate the minimal tolerable entry dose (MTED), the maximal tolerable dose (MTD) and the number of updosing steps in a updosing regimen (UDR) by conducting a human clinical trial.

A dose is considered tolerable if, following intake of the last tablet of a dose-step, the individual has not experienced any treatment-related systemic adverse events, although the individual may have experienced treatment-related adverse events, but these are at the most local reactions of moderate intensity and the individual may have experienced adverse events that are not treatment-related which should not be considered in the assessment of tolerability.

Dose tolerability is meant to designate a binary endpoint, where an individual is classified as having either tolerated or not tolerated a specific dose of the trial medication (peanut SLIT-tablet). A dose is considered tolerated when pre-selected individual tolerability criteria has been met. A not tolerated dose can occur for several reasons, either that the pre-selected individual tolerability criteria has not been met, the individual met the individual stopping criteria or discontinued treatment due to adverse events.

The individual stopping criteria meaning that an individual should immediately discontinue treatment are met when, over the duration of the trial, the individual experience a cumulative total of two or more of the following events at distinct timepoints: a) a severe systemic allergic reaction, b) requiring treatment with epinephrine, c) a severe local swelling compromising respiration and d) in the opinion of the investigator, it is not safe to continue treatment.

A secondary objective is to evaluate the safety of the peanut SLIT-tablet. Safety parameters investigated are: a) treatment emergent adverse events (TEAEs), b) events of special interest (ESIs), c) systemic allergic reactions, d) events treated with epinephrine, e) severe swelling or oedema of the mouth and/or throat, f) severe asthma exacerbations, g) Eosinophilic esophagitis (EoE), h) local application site reactions, i) discontinuation of treatment due to TEAEs, j) occurrence of clinically significant findings in oropharyngeal examination, physical examination, vital signs, and clinical laboratory values, k) changes from baseline in vital signs and clinical laboratory values.

The following determinations may be made: An overall summary of TEAEs may present the number (%) of individuals experiencing an event and the number (%) of events for all TEAEs and by causality, severity, seriousness, action taken, outcome, whether event was leading to discontinuation and whether medication was given. The above summaries may be repeated for treatment-related TEAEs, serious treatment-related TEAEs and treatment-related TEAEs leading to discontinuation of IMP. TEAEs may be determined by dose, by age-group and by time to onset.

For each ESI, an overall summary and a summary by patient groups, age or dose may be made. The overall summary of systemic allergic reactions may also include anaphylaxis as an additional severity category. For systemic allergic reactions, time to onset and duration of event may be summarised. Local application site reactions may be reported including time to onset and duration of the site reaction may be summarised.

A third objective is to evaluate the effect of the peanut SLIT-tablet updosing regimen on immunological parameters, such as specific IgE, IgG4, IgG, IgG1 or IgA against peanut or against specific peanut allergens (Ara h 1, Ara h 2, Ara h 3 or Ara h 6). Peanut SPT wheal diameter before start of treatment and during or after end of updosing regimen may also be included.

### Trial Medication

The trial medication contains a drug substance obtained by the manufacturing processes of examples 2 to example 5, and the trial medication is further formulated as an oral lyophilisate (Peanut SLIT-tablet) that instantly disintegrates when placed under the tongue. The drug substance comprises the four key peanut allergens: nAra h 1, nAra h 2, nAra h 3 and nAra h 6 in standardised amounts (target is to achieve equimolar amounts between the four allergens). Other peanut allergens typically present in raw peanuts are also present in the drug substance. The peanut SLIT-tablet is made with different potency doses of peanut protein, such as 1 DU, 3 DU, 10 DU, 40 DU, 120 DU, 360 DU, 1080 DU, 2160 DU or 4320 DU. The potency unit (DU) is defined as 1 μg peanut protein of a reference batch of the trial medication, which has been produced by the same process as the actual trial medication and with the same pre-determined levels of the four key peanut allergens. The peanut protein content was determined by amino acid analysis. The allergens nAra h 1, nAra h 2, nAra h 3 and nAra h 6 constituted about 85% to 95% by weight of the peanut proteins.

### Individuals

Individuals eligible for the trial are adults (18-65 years), adolescents (12-17 years) and children (4-11 years) with a clinical history of peanut allergy. Peanut allergy diagnosis is determined by a clinical history of peanut allergy, IgE sensitisation determined by serum allergen-specific IgE, a positive SPT reaction to peanut, and a positive reaction in a DBPCFC. All enrolled individuals must go through a double-blind, placebo-controlled, food challenge (DBPCFC) and experience dose-limiting symptoms at or before the 100 mg peanut protein challenge dose to confirm peanut allergy. Among the enrolled individuals, highly sensitised individuals will be defined as those experiencing dose-limiting symptoms at either the 1 mg or 3 mg challenge doses of peanut protein in the screening DBPCFC. DBPCFC consists of two separate Oral Food Challenges (OFCs) performed with either peanut protein or placebo, which are administered in a random order and blinded for the physician conducting the OFC. Each OFC is performed by ingestion of gradually increasing amounts of either peanut protein (usually consisting of standardised defatted peanut flour) or placebo mixed into a vehicle food until pre-defined dose-limiting symptoms are observed. The dose increments are 1 mg, 3 mg, 10 mg, 30 mg and 100 mg. The dose-limiting symptoms will be assessed by evaluation of the tolerability of each challenge dose based on the severity of allergic symptoms elicited at a particular dose. The severity of an allergic reaction will be determined by the investigator as the maximum severity of any observed symptoms. The CoFAR grading system for OFCs (Chinthrajah et al. 2022) can be used as general guide. Vital signs (blood pressure and pulse rate) will be measured just prior to each challenge dose or at appropriate times between challenge doses, if the dosing interval is prolonged. Assessment for signs and symptoms of an allergic reaction will be performed at the time that vital signs are checked.

### Severity Grading of Allergic Symptoms During the DBPCFC According to (Chinthrajah et al. 2022):

| Mild | Moderate | Severe |
|---|---|---|
| Skin: limited (few) or | Skin: systemic hives | Skin: severe |
| localised hives, swelling | (e.g. numerous or | generalised urticaria/ |
| (e.g. mild lip oedema), | widespread hives), | angioedema/erythema |
| skin flushing (e.g. few | swelling (e.g. | |
| areas of faint erythema) | significant lip or face | |
| or mild pruritus (e.g. | oedema), pruritus | |
| occasional scratching) | causing protracted | |
| | scratching, more than | |
| | a few areas of | |
| | erythema or | |
| | pronounced erythema | |
| Respiratory: rhinorrhoea | Respiratory: throat | Respiratory: laryngeal |
| (e.g. occasional sniffling | tightness without | oedema, throat |
| or sneezing), nasal | hoarseness, persistent | tightness with |
| congestion, occasional | cough, wheezing | hoarseness, wheezing |
| cough, throat discomfort | without dyspnoea | with dyspnoea, stridor |
| GI: mild abdominal | GI: persistent | GI: severe abdominal |
| discomfort (including | moderate abdominal | pain/cramping/ |
| mild nausea with or | pain/cramping/nausea | repetitive vomiting |
| without decreased | with decreased | |
| activity), isolated emesis | activity, vomiting | |
| thought to be secondary | | |
| to gag | | |
| | | Neurological: change |
| | | in mental status |
| | | Circulatory: |
| | | clinically significant |
| | | hypotension |

Further evaluation will include:
No symptoms: If a dose elicits no symptoms, the dose will be assessed as tolerated.
Mild symptoms: A reaction characterised by only mild symptom(s), will be assessed as tolerated or not tolerated based on the investigator's clinical judgement. The below examples serve as guidance for when a dose eliciting only mild symptoms may be considered to be tolerated:
   Symptoms are isolated to a single organ system
   Symptoms resolve with no pharmaceutical intervention or with a single oral administration of an H1 antihistamine
   Symptoms do not require administration of epinephrine
   Symptoms are not worsening in intensity or distribution over time
   Symptoms resolve, or show definite signs of resolving, in under 1 hour
   Symptoms do not include objective wheezing
   If an allergic reaction to dosing is characterised by mild symptoms that do not meet all of the above criteria then the dose should be assessed as not tolerated. If dose-eliciting mild symptoms do not fit all of the above criteria and the dose is assessed as tolerated, an explanation as to why the dose was considered tolerated must be recorded in the eCRF.
Moderate symptoms: A reaction characterised by moderate symptoms will in general be assessed as not tolerated. On rare occasions, dose-eliciting moderate symptoms could be assessed as tolerated if symptoms are transient, self-limiting (requiring no intervention and resolving completely), and affecting only a single organ system. Typically, such symptoms would be subjective only. Any dose associated with moderate symptoms and assessed as tolerated must be accompanied by an explanation in the eCRF as to why the dose was considered tolerated.
Severe symptoms: A reaction characterised by severe symptoms will be assessed as not tolerated.

### Treatment Outline

The individuals will initially be selected for once-daily sublingual treatment of a fixed-dose peanut protein dose (such as either 1 DU, 3 DU, 10 DU, 40 DU, or 120 DU) of the peanut SLIT-tablet for 2 weeks. Following the two weeks treatment, the treatment safety and dose tolerability will be evaluated.

Then individuals will be selected for the investigation of the treatment safety and dose tolerability of an updosing regimen (UDR) including different age cohorts (adults 18-65 years, adolescents 12-17 years and children 4-11 years) and whether the individual is highly sensitive to peanut or not.

The UDR consists of series of once-daily fixed-dose administration of the same dose to the sublingual mucosa for two weeks and wherein the dose is increased every two weeks. Individuals will start the UDR at an entry dose identical to the minimal entry dose determined in the initial two-week trial (such as either be 1 DU, 3 DU, 10 DU, 40 DU, or 120 DU). Subsequent dose steps in the UDR will follow every two weeks (such as 360 DU, 1080 DU, 2160 DU and 4320 DU peanut protein) to investigate the maximal dose tolerated by the study individuals, eventually determined by age group. Individuals will updose until they either complete the planned UDR (up to dose 4320 μg) or reach their individual MTD. Flexibility in the UDR is allowed; each dose-step can be extended with an additional 1 week of treatment if the individual does not meet the individual updosing criteria after 2 weeks.

The MTED dose will be selected on the basis of doses where at least 75% of the individuals included in the assessment of dose tolerability for that dose meet the prerequisite for being able to tolerate the dose.

The dose tolerability will be assessed in that the number of individuals included in the assessment of dose tolerability and the number (%) of individuals that tolerated and did not tolerate each dose will be summarised.

### Assessment of Tolerability

Expected adverse reactions of the peanut SLIT-tablet include local allergic reactions in the mouth and throat of mild or moderate severity. As with any AIT, there is a risk of more severe AEs, such as severe systemic allergic reactions and severe swelling of the mouth and throat, which could compromise airways.

For an individual, a dose is considered tolerable if, following intake of the last tablet of the dose step:
Any treatment-related AEs are local application site reactions no more than mild to moderate in intensity in that local application site reactions are defined as treatment-related adverse events that occur in close proximity to the sublingual tablet application site in and around the mouth, throat, ear, nose, eyes and upper gastrointestinal tract, with an onset within 60 min of tablet intake.
The occurrence of any treatment-related systemic AE following intake of the last tablet of the dose step would mean that the dose has not been tolerated.
Treatment-related AEs occurring prior to the intake of the last tablet of the dose step and AEs that are not treatment-related should not be considered in the assessment of tolerability.

An individual is permitted to updose if, after intake of the last tablet on their current dose step:
The individual meets the individual tolerability criteria; AND

They meet the individual pre-dosing safety check; AND it is safe to do so, in the opinion of the investigator

If an individual does not meet all the individual updosing criteria after 2 weeks treatment, they may continue treatment with the current dose for 1 additional week (i.e. extended treatment).

However, individuals that do not meet the individual IMP compliance criteria after 2 weeks treatment may only receive extended treatment if they have received treatment on at least 4 of the previous 7 days. Individuals that do not meet this additional criterion will be unable to meet the compliance criteria after extended treatment and have therefore completed treatment according to protocol-specified rules and criteria.

If an individual does not meet all the individual updosing criteria after extended treatment, then the individual has completed treatment according to protocol-specified rules and criteria.

### Assessment of Safety

For characterising the safety profile of peanut SLIT-tablet, unsolicited AEs, including ESIs, and solicited AEs are collected for the duration of the trial. The solicited AEs are based on local application site reactions to SLIT-tablets (Canonica et al. 2014). In addition, questions regarding hives and dyspnea have been included to monitor for possible systemic allergic reactions and asthma exacerbations. The solicited AEs are reported based on 16 pre-specified symptoms that the individual will be asked to record daily in an eDiary. The symptoms will be evaluated by the investigator and reported in the eCRF as solicited AEs, if applicable.

### AEs

An AE is any untoward medical occurrence in a clinical trial individual and which does not necessarily have a causal relationship with the administered IMP. An AE can therefore be any unfavourable and unintended sign (including e.g. an abnormal laboratory finding or medication error), symptom, or disease, whether or not considered related to the IMP.

### SAEs

An SAE is any untoward medical occurrence or effect that:
Results in death
Is life-threatening (this refers to an event in which the individual was at risk of death at the time of the event; it does not refer to an event that hypothetically might have caused death if it had been more severe)
Requires in-subject hospitalisation, regardless of duration, or prolongation of existing hospitalisation
Results in persistent or significant disability or incapacity
Is a congenital anomaly or birth defect
Is judged to be medically important (this refers to an event that may not be immediately life-threatening or result in death or hospitalisation, but may jeopardise the individual or may require intervention to prevent one of the other outcomes listed above)

### Local Application Site Reactions

Local application site reactions are treatment-related adverse events that occur in close proximity to the application site of the SLIT-tablet with a temporal relationship to administration of the tablet. For the SLIT-tablet, this means AEs that initiate within 60 minutes of tablet intake and occur in and around the mouth, throat, ear, nose, eyes and upper gastrointestinal tract. Ear, eye, and nasal symptoms are included as neurogenic reflexes originating from the application site which may trigger symptoms like sneezing, runny nose, ear itching etc. Upper GI symptoms may occur from swallowing small amounts of allergen.

For SLIT-tablet administration, local application site reactions include:
Ear/eye/nose: itching, runny nose or eye
Mouth/tongue/lips: itching, tingling, swelling, pain, ulcer
Throat: itching, irritation, cough, tightness, swelling
Skin: localised flushing/hives in the head/neck area (hives affecting a single dermatome)
Gastrointestinal: upper abdominal pain, nausea, single episode of vomiting, single episode of diarrhoea
Local application site reactions of mild or moderate intensity are considered acceptable when assessing the individual tolerability criteria.

### Solicited Adverse Events (SAEs)

Sixteen pre-specified symptoms will be solicited via the eDiary after each tablet intake throughout the trial. The first 14 pre-specified symptoms are identified as potential local application site reactions to SLIT tablets (Canonica et al. 2014). In addition, questions regarding hives and dyspnoea have been included to monitor for possible systemic allergic reactions and asthma exacerbation:
A sore in the mouth (canker sore)
Swelling in the mouth
Swelling of the lips
Swelling of the tongue
Throat swelling
Itching in the mouth
Itching in the ear
Pain in mouth
Pain in tongue
Throat irritation/tickle
Stomach pain
Nausea
Vomiting
Diarrhoea
Hives over the body
Shortness of breath

### ESIs

Selected AEs (non-serious or serious) will be considered ESIs. ESIs are events that are considered critical for the evaluation of the product's safety profile and for which additional data will be collected on a separate eCRF form. The ESIs for this trial are:

### Systemic Allergic Reactions:

Events treated with epinephrine
Severe local swelling or oedema of the mouth and/or throat
Severe asthma exacerbations
EoE

All adverse events entered in the eCRF as ESIs by the investigator will be classified as ESIs. Additional searches of the safety database will be conducted to identify other potential ESIs.

The term ‘systemic allergic reaction’ is used to define a clinical syndrome that covers a range of allergic symptoms. These symptoms occur as part of a clinical continuum from mild symptoms to rapidly progressing life-threatening symptoms.

The combination of symptoms defining a systemic allergic reaction should be graded by the investigator into grade 1, 2, or 3:
Grade 1: Mild symptoms, such as (skin & subcutaneous tissues or mild respiratory), Generalised pruritus/hives/urticaria; angioedema; mild wheezing, mild dyspnoea; tachycardia (increase >15 beats/min from baseline value)
Grade 2: Moderate symptoms, such as (mild symptoms and features suggesting moderate respiratory, cardiovascular or GI symptoms), moderate dysphagia, hoarseness and/or stridor, shortness of breath, moderate dyspnoea moderate wheezing & retractions; more than one episode of vomiting and/or diarrhoea; mild dizziness.
Grade 3: Severe symptoms, such as hypoxia, hypotension, or neurological compromise, Cyanosis or SpO2<92% at any stage, respiratory compromise, hypotension, confusion/collapse, loss of consciousness; loss of bowel control.

These symptoms are examples, and an individual could represent with symptoms from several stages. The most severe symptom present should determine the severity stage.

Anaphylaxis is defined as: a potentially life-threatening severe systemic allergic reaction involving two or more organ systems, which include either cardiovascular and/or respiratory compromise. Respiratory compromise describes a deterioration in respiratory function with a high likelihood of rapid progression to respiratory failure and death.

The definition of anaphylaxis is based on the definition by (Sampson H. A. et al. 2006 and may be characterised by: Acute onset of an illness (minutes to several hours) with involvement of the skin and/or mucosal tissue (e.g., generalised hives, pruritus, or flushing, or swollen lips, tongue, or uvula) AND at least one of the following:
Respiratory compromise (e.g., dyspnoea, wheeze-bronchospasm, stridor, reduced PEF, hypoxemia)
Reduced blood pressure (BP) or associated symptoms of end-organ dysfunction (e.g., hypotonia [collapse], syncope, incontinence)
Reduced BP: Children: low systolic BP (age specific) or greater than 30% decrease in systolic BP. Adults: systolic BP of less than 90 mm Hg or greater than 30% decrease from that person's baseline value.
Events treated with epinephrine
Events for which epinephrine is administered independent of route.
Severe swelling or oedema of the mouth and/or throat
A severe swelling or oedema of the mouth and/or throat is defined as a swelling causing respiratory compromise requiring treatment intervention with epinephrine. If epinephrine is used as a preventive treatment measure, it will not automatically count as a treatment intervention that defines the swelling as severe.
Severe asthma exacerbations
A severe asthma exacerbation is defined as:
   Use of systemic corticosteroids for treatment of asthma symptoms for at least 3 days, or
   Hospitalisation for more than 12 hours due to asthma requiring treatment with systemic corticosteroids

### EoE

Events of EoE based on a biopsy showing 15 eosinophils/HPF or confirmed by a gastroenterologist will be classified as ESI.

Significant Laboratory event

A significant laboratory event should be recorded as an AE if one of the following is applicable:
It is abnormal and clinically significant (medical judgement by investigator)
It leads to a change or discontinuation of treatment with IMP
It fulfils a seriousness criterion
It indicates a potential safety risk to the individual
In case of elevated AST or ALT value ≥3 times the normal upper limit and bilirubin ≥2 times upper normal limit and alkaline phosphatase is not >2 times upper normal limit (Hy's law)
Medication errors, including overdose, abuse and misuse of the IMP
Medication error, misuse, overdose and abuse of the IMP must always be collected in line with adverse event reporting, with or without associated AEs.
Medication error: Any unintended failure in the medication treatment process that leads to, or has the potential to lead to, harm to the individual
Overdose: Any cumulative dose taken in one day that exceeds the dose intended by this protocol, regardless of whether the dose has caused any AEs
Abuse: Persistent or sporadic, intentional excessive use which is accompanied by harmful physical or psychological effects
Misuse: Intentional and inappropriate use

### AE Assessments

The severity of an AE is a clinical observation assessed by the investigator using the following definitions:
Mild: Transient symptoms, no interference with the individual's daily activities
Moderate: Marked symptoms, moderate interference with the individual's daily activities
Severe: Considerable interference with the individual's daily activities, unacceptable

The causal relationship between an AE and the IMP is assessed by the investigator using the following definitions:
Possible: A reasonable possibility of a causal relationship between the event and the IMP.
Unlikely: The event is most likely caused by a different aetiology than the IMP.
For SAEs assessed as unlikely and possible related to IMP, the most likely alternative aetiology should be provided.

The outcome of an AE is assessed by the investigator using the following definitions:
Recovered: Fully recovered or the condition has returned to baseline
Recovered with sequelae: As a result of the AE the individual suffered persistent disability/incapacity. If the sequelae meet an SAE criterion, the AE must be reported as an SAE
Not recovered: The condition has not returned to baseline, however, symptoms may have improved
Fatal: Event that results in death
Unknown: The outcome is unknown. This term should only be used when no other definition is possible e.g. the individual is lost to follow-up.

At each contact with the trial site, the individual must be asked about AEs in an objective manner such as “Have you experienced any problems since the last contact?”

AEs must be recorded on the AE form. One single AE form must be used per AE from start to resolution. For SAEs and ESIs, specific SAE and ESI forms in the eCRF must also be filled in.

In case of concomitant medication for treatment of the AE, the concomitant medication form must be filled in.

### LIST OF REFERENCES

Becker, W. M., et al. “Peanut Allergens: New Consolidated Findings on Structure, Characteristics, and Allergome.” Allergologie Select, vol. 2, no. 1, 0 2018, pp. 67-79, doi:10.5414/ALX01418E.
Blanc, Fany, et al. “Boiling Peanut Ara h 1 Results in the Formation of Aggregates with Reduced Allergenicity.” Molecular Nutrition & Food Research, vol. 55, no. 12, 2 Dec. 2011, pp. 1887-94, doi:10.1002/mnfr.201100251.
Boldt, Andrea, et al. “Analysis of the Composition of an Immunoglobulin E Reactive High Molecular Weight Protein Complex of Peanut Extract Containing Ara h 1 and Ara h 3/4.” PROTEOMICS, vol. 5, no. 3, John Wiley & Sons, Inc, 0 February 2005, pp. 675-86, doi:10.1002/pmic.200401150.
Breiteneder H, Chapman M D. Allergen Nomenclature. In Allergens and Allergen Immunotherapy: Subcutaneous, sublingual and oral. 5th Edition. Edited by Richard F. Lockey and Dennis K. Ledford. CRC Press, Taylor and Francis Group, Boca Raton, Florida, USA, 2014, pp 37-49.
Burk et al. Sublingual immunotherapy for peanut allergy: Long-term follow-up of a randomised multicenter trial. Journal of Allergy and Clinical Immunology, 2015, Vol 135, p 1240-1248.e3. DOI: 10.1016/j.jaci.2014.12.1917.
Canonica, et al. “Sublingual Immunotherapy: World Allergy Organization Position Paper 2013 Update.” World Allergy Organization Journal, vol. 7, Lippincott Williams and Wilkins, 0 2014, doi:10.1186/1939-4551-7-6.
Chinthrajah, et al. “Sustained Outcomes in Oral Immunotherapy for Peanut Allergy (POISED Study): A Large, Randomised, Double-Blind, Placebo-Controlled, Phase 2 Study.” Lancet, The, vol. 394, no. 10207, Elsevier, 19 Oct. 2019, pp. 1437-49, doi:10.1016/S0140-6736(19)31793-3.
Chinthrajah, J et al. Updating the CoFAR Grading Scale for Systemic Allergic Reactions in Food Allergy. Journal of Allergy and Clinical Immunology, 2022, vol 149, p AB107, DOI: 10.1016/j.jaci.2021.12.372.
Davis, Carla M., et al. “Maximum Dose Food Challenges Reveal Transient Sustained Unresponsiveness in Peanut Oral Immunotherapy (POIMD Study).” Journal of Allergy and Clinical Immunology: In Practice, The, vol. 10, no. 2, Elsevier, 0 February 2022, pp. 566-576.e6, doi:10.1016/j.jaip.2021.10.074.
De Angelis, Elisabetta, et al. “Effects of the Varietal Diversity and the Thermal Treatment on the Protein Profile of Peanuts and Hazelnuts.” Journal of Food Quality, vol. 2018, Blackwell Publishing Inc, 0 2018, pp. 1-10, doi:10.1155/2018/7635957.
Durham, Stephen R.; Shamji, Mohamed H. Allergen immunotherapy: past, present and future. Nature Reviews Immunology, 2022 pages: 1-12, DOI: 10.1038/s41577-022-00786-1.
Hemmings, et al. “Ara h 2 Is the Dominant Peanut Allergen despite Similarities with Ara h 6.” Journal of Allergy and Clinical Immunology, The, vol. 146, no. 3, Mosby, 0 September 2020, pp. 621-630.e5, doi:10.1016/j.jaci.2020.03.026.
Iqbal, Amjad, et al. “Allergens of Arachis Hypogaea and the Effect of Processing on Their Detection by ELISA.” Food & Nutrition Research, vol. 60, no. 1, Taylor & Francis, 1 Jan. 2016, doi:10.3402/fnr.v60.28945.
Kim, E. H., et al. “Sublingual Immunotherapy for Peanut Allergy: Clinical and Immunologic Evidence of Desensitization.” Journal of Allergy and Clinical Immunology, The, vol. 127, no. 3, Mosby, 0 March 2011, pp. 640-646.e1, doi:10.1016/j.jaci.2010.12.1083.
Koppelman, Stef, et al. “Release of Major Peanut Allergens from Their Matrix under Various PH and Simulated Saliva Conditions-Ara H2 and Ara H6 Are Readily Bio-Accessible.” Nutrients, vol. 10, no. 9, MDPI, 11 September 2018, p. 1281, doi:10.3390/nu10091281.
Koppelman, Stef J., et al. “Allergenicity Attributes of Different Peanut Market Types.” Food and Chemical Toxicology, vol. 91, Pergamon, 0 May 2016, pp. 82-90, doi:10.1016/j.fct.2016.02.016.
Krause, Maren, et al. “Human Monocyte-Derived Type 1 and 2 Macrophages Recognize Ara h 1, a Major Peanut Allergen, by Different Mechanisms.” Scientific Reports, vol. 11, no. 1, Nature Publishing Group, 12 May 2021, doi:10.1038/s41598-021-89402-1.
Kukkonen, A. K., et al. “Ara h 2 and Ara 6 Are the Best Predictors of Severe Peanut Allergy: A Double-blind Placebo-controlled Study.” Allergy, vol. 70, no. 10, John Wiley & Sons, Inc, 25 September 2015, pp. 1239-45, doi:10.1111/all.12671.
Maleki, Soheila J., et al. “Differences among Heat-Treated, Raw, and Commercial Peanut Extracts by Skin Testing and Immunoblotting.” Annals of Allergy, Asthma & Immunology, vol. 105, no. 6, Elsevier, 0 December 2010, pp. 451-57, doi:10.1016/j.anai.2010.09.025.
Marsh J et al. Purification and characterisation of a panel of peanut allergens suitable for use in allergy diagnosis. Mol Nutr Food Res. 2008 November; 52 Suppl 2: S272-85. doi: 10.1002/mnfr.200700524.
Palladino, and Breiteneder. “Peanut Allergens.” Molecular Immunology, vol. 100, Elsevier, 0 August 2018, pp. 58-70, doi:10.1016/j.molimm.2018.04.005.
Pandey, Arun K., et al. “Hypoallergen Peanut Lines Identified Through Large-Scale Phenotyping of Global Diversity Panel: Providing Hope Toward Addressing One of the Major Global Food Safety Concerns.” Frontiers in Genetics, vol. 10, Frontiers Research Foundation, 0 2019, p. 1177, doi:10.3389/fgene.2019.01177.
Poms, Roland E., et al. “Effect of Roasting History and Buffer Composition on Peanut Protein Extraction Efficiency.” Molecular Nutrition & Food Research, vol. 48, no. 6, 0 November 2004, pp. 459-64, doi:10.1002/mnfr.200400052.
Restani, Patrizia, et al. “Identification of the Basic Subunit of Ara h 3 as the Major Allergen in a Group of Children Allergic to Peanuts.” Annals of Allergy, Asthma & Immunology, vol. 94, no. 2, Elsevier, 0 February 2005, pp. 262-66, doi:10.1016/S1081-1206(10)61306-3.
Sampson, H. A., A. Munoz-Furlong, et al. “Second Symposium on the Definition and Management of Anaphylaxis: Summary Report-Second National Institute of Allergy and Infectious Disease/Food Allergy and Anaphylaxis Network Symposium.” Annals of Emergency Medicine, vol. 47, no. 4, Mosby, 0 April 2006, pp. 373-80, doi:10.1016/j.annemergmed.2006.01.018.
Sampson, H. A., R. Gerth van Wijk, et al. “Standardizing Double-Blind, Placebo-Controlled Oral Food Challenges: American Academy of Allergy, Asthma & Immunology-European Academy of Allergy and Clinical Immunology PRACTALL Consensus Report.” Journal of Allergy and Clinical Immunology, The, vol. 130, no. 6, Mosby, 0 December 2012, pp. 1260-74, doi:10.1016/j.jaci.2012.10.017.
Spiric, J., et al. “Mass Spectrometry to Complement Standardization of House Dust Mite and Other Complex Allergenic Extracts.” Clinical & Experimental Allergy, vol. 47, no. 5, John Wiley & Sons, Inc, 27 Apr. 2017, pp. 604-17, doi:10.1111/cea.12931.
Valk, J., et al. “Mono-Sensitisation to Peanut Component Ara h 6: A Case Series of Five Children and Literature Review.” European Journal of Pediatrics, vol. 175, no. 9, Springer, 0 September 2016, pp. 1227-34, doi:10.1007/s00431-016-2733-7.
van Boxtel, Evelien L., et al. “Allergen Ara h 1 Occurs in Peanuts as a Large Oligomer Rather Than as a Trimer.” Journal of Agricultural and Food Chemistry, vol. 54, no. 19, American Chemical Society, 1 September 2006, pp. 7180-86, doi:10.1021/jf061433+.
Vereda, A., et al. “Peanut Allergy: Clinical and Immunologic Differences among Patients from 3 Different Geographic Regions.” Journal of Allergy and Clinical Immunology, The, vol. 127, no. 3, Mosby, 0 March 2011, pp. 603-07, doi:10.1016/j.jaci.2010.09.010.
Vickery, B. P et al. Peanut oral immunotherapy modifies IgE and IgG″4 responses to major peanut allergens. Journal of Allergy and Clinical Immunology, 2013, vol. 131, p 128-134. DOI: 10.1016/j.jaci.2012.10.048.
Wang, Shuo, et al. “Effect of Roasted Peanut Allergen Ara h 3 Protein on the Sensitization of Caco-2 Cells.” Journal of the Science of Food and Agriculture, vol. 101, no. 13, John Wiley & Sons, Ltd., 4 Sep. 2021, pp. 5325-36, doi:10.1002/jsfa.11180.
Wunschmann S et al. (2019). Peanut flour protein with defined allergen content for use as a reference standard. European academy of allergy immunology congress, Vol 74, S106, p 475-476. Doi:10.1111/all.13961
Zhang, Wenming, et al. “Comparison of Sublingual Immunotherapy and Oral Immunotherapy in Peanut Allergy.” Allergo Journal International, vol. 27, no. 6, SpringerMedizin, 0 September 2018, pp. 153-61, doi:10.1007/s40629-018-0067-x.

1. A pharmaceutical composition comprising each of the peanut proteins nAra h 1, nAra h 2, nAra h 3, and nAra h 6 and further comprises a pharmaceutically acceptable carrier, diluent, excipient, or vehicle, wherein
i. the molar concentration of nAra h 2 is in the range of 2 to 12 nmol/mg of the total mass of peanut proteins;
ii. the molar ratio of each of the pairs nAra h 1:nAra h 2, nAra h 3:nAra h 2 and nAra h 6:nAra h 2 is in the range of 0.5 to 2.0; and
iii. the pharmaceutical composition is a sublingual solid dosage form,
wherein each of the peanut proteins nAra h 1, nAra h 2, nAra h 3, and nAra h 6 have been obtained by extraction of defatted and pulverised raw peanut kernels by an aqueous solvent having pH in the range of 7 to 9; and
wherein the molar ratio is determined using the molar concentration of the monomeric polypeptide of nAra h 1, nAra h 2, nAra h 3, and nAra h 6 as determined by reverse phase HPLC and by using the average molecular mass of 68757 g/mol for Ara h 1, 17994 g/mol for Ara h 2, 58600 g/mol for Ara h 3 and 14846 g/mol for Ara h 6 for converting weight concentrations to molar concentrations.

2. The pharmaceutical composition according to claim 1, wherein the molar ratio of each of the pairs nAra h 1:nAra h 2, nAra h 3:nAra h 2 and nAra h 6:nAra h 2 is in the range of 0.5 to 1.5.

3. (canceled)

4. The pharmaceutical composition according to claim 1, wherein nAra h 1, nAra h 2, nAra h 3, and nAra h 6 in combination constitutes at least 80% by weight of the total peanut protein in the composition.

5-6. (canceled)

7. The pharmaceutical composition according to claim 1, wherein the concentration of each of nAra h 1, nAra h 3 and nAra h 6 is in the range of 2 to 12 nmol/mg of the total mass of peanut proteins.

8-10. (canceled)

11. The pharmaceutical composition according to claim 1, wherein the reverse phase HPLC comprises mixed isocratric and gradient elution, which comprises mixing an eluent A consisting of aqueous 0.1% trifluoroacetic acid with increasing volumes of an eluent B consisting of acetonitrile mixed with 0.1% trifluoroacetic acid.

12-13. (canceled)

14. The pharmaceutical composition according to claim 1 being essentially free from peanut protein having a molecular mass of >700 kDa.

15. The pharmaceutical composition according to claim 1, further comprising a peanut protein selected from the group consisting of Ara h 5, Ara h 7, Ara h 8, Ara h 9, Ara h 10, Ara h 11, Ara h 12, Ara h 13, Ara h 14, Ara h 15, Ara h 16, Ara h 17 and Ara h 18.

16. The pharmaceutically composition according to claim 1, wherein the pharmaceutically acceptable carrier, diluent, excipient, or vehicle forms a solid sublingual unit dosage form selected from the group consisting of a compressed tablet, a non-compressed tablet, a film, a paste or an unit dose lyophilizate.

17-23. (canceled)

24. A method of mitigating peanut allergy and/or peanut-induced anaphylaxis by allergen-specific immunotherapy, comprising administering a therapeutically effective dose of the pharmaceutical composition according to claim 1.

25-33. (canceled)

34. A method of mitigation of peanut allergy and/or peanut allergen-induced anaphylaxis in a human individual by conducting allergen-specific immunotherapy, wherein the method comprises an updosing phase and optionally a maintenance phase, wherein the updosing phase comprises multiple consecutive series of sublingually administering a daily dose of peanut protein composition comprises each of nAra h 1, nAra h 2, nAra h 3, and nAra h 6,
wherein the daily dose within each series of the updosing phase is identical; any dose in a preceding series is lower than in a subsequent series; and each series has a duration ranging from 6 to 30 days; and wherein
the daily dose of peanut protein of a first series is in the range of 1 μg to 150 μg;
the daily dose of peanut protein in the last series in the range of 300 μg to 5000 μg; and
wherein the total number of series is in the range from 3 to 7.

35. The method according to claim 34, wherein the peanut proteins have been extracted from defatted and pulverised raw peanut kernels by an aqueous solvent having pH in the range of 7 to 9 and the extracted peanut proteins comprises each of nAra h 1, nAra h 2, nAra h 3, and nAra h 6.

36. The method according to claim 34, wherein the peanut proteins comprises a molar ratio of each of the pairs nAra h 1:nAra h 2, nAra h 3:nAra h 2, and nAra h 6:nAra h 2 in the range 0.5 to 2.0, wherein the molar ratio is determined using the concentration of the monomeric polypeptides of each of nAra h 1, nAra h 2, nAra h 3, and nAra h 6.

37. The method according to claim 34, wherein the peanut proteins comprise an amount of nAra h 2 ranging from 50-150 μg per mg peanut protein.

38. The method according to claim 34, wherein the peanut protein comprises an amount of nAra h 3 ranging from 160-500 μg per mg peanut protein.

39. The method according to claim 34, wherein nAra h 1, nAra h 2, nAra h 3, and nAra h6 in combination constitutes at least 75% by weight of the peanut protein.

40. The method according to claim 34, wherein each of the series has a duration of 10-21 days.

41. The method according to claim 34, wherein the daily peanut protein dose of a series later than the first series is increased by a factor of 2 to 4 compared to the daily dose of the directly preceding series.

42-50. (canceled)

51. A method for preparing a peanut protein composition comprising the peanut proteins—nAra h 1, nAra h 2, nAra h 3, and nAra h 6, the method comprising the following steps:
1) providing an extract of peanut proteins, said extract is obtained by extracting defatted and pulverised raw peanut kernels with an aqueous buffer having pH in the range of 7-8 to obtain an aqueous extract comprising each of nAra h 1, nAra h 2, nAra h 3, and nAra h 6;
2) subjecting said aqueous extract comprising each of nAra h 1, nAra h 2, nAra h 3, and nAra h 6 to anion exchange chromatography using a strong anion exchange material with stepwise or continuous aqueous salt gradient elution at pH in the range of 7 to 8, whereby each of nAra h 1, nAra h 2, nAra h 3, and nAra h 6 are eluted and collected into fractions individually enriched for nAra h 1, nAra h 2, nAra h 3, or nAra h 6; and
3) optionally collecting a flow-through fraction from the anion exchange chromatography; and
4) combining the enriched fractions or aliquots thereof as obtained in step 2 or in combined step 2 and 3 to obtain said peanut protein composition.

52. The method according to claim 51, wherein the aqueous buffer of step 1 comprises TRIS in a molar range of 10 to 200 mM and NaCl in an amount in the range of 5 to 200 mM.

53. The method according to claim 51, wherein the salt of step 2 is NaCl.

54. The method according to claim 51, wherein the concentrations of monomeric polypeptides of nAra h 1, nAra h 2, nAra h 3, and nAra h 6 in the individual fractions obtained in step 2 are controlled before being combined in step 4.

55. The method according to claim 51, wherein the combined fractions or aliquots thereof are combined to obtain peanut compositions comprising a molar ratio of each of the pairs nAra h 1:nAra h 2, nAra h 3:nAra h 2, nAra h 6 nAra h 2 in the range of 0.5 to 2.0, wherein the molar ratio is determined using the concentration of monomeric polypeptides of each of nAra h 1, nAra h 2, nAra h 3, and nAra h 6.

56. The method according to claim 51, wherein the flow-through fraction or an aliquot thereof as obtained in step 3 is combined with the combined fractions of step 2.

57. The method according to claim 51, wherein the peanut protein composition further comprises a peanut protein selected from the group consisting of Ara h 5, Ara h 7, Ara h 8, Ara h 9, Ara h 10, Ara h 11, Ara h 12, Ara h 13, Ara h 14, Ara h 15, Ara h 16, Ara h 17 and Ara h 18.

58. The method according to claim 51, wherein the fractions collected in step 2 are essentially free from peanut protein having a molecular mass of >700 kDa.
